(19) United States
(12) Patent Application Publication
(10) Pub. No.: US 2024/0368157 A1
(43) Pub. Date: Nov. 7, 2024

(54) FORMS AND COMPOSITIONS OF A JAK2 INHIBITOR

(71) Applicant: Ajax Therapeutics, Inc., New York, NY (US)

(72) Inventors: Craig E. Masse, Cambridge, MA (US); Jeremy R. Greenwood, Brooklyn, NY (US); Jiayi Xu, Marlboro, NJ (US); Sayan Mondal, New York, NY (US); Phani Ghanakota, Edison, NJ (US)

(21) Appl. No.: 18/653,857

(22) Filed: May 2, 2024

Related U.S. Application Data
(60) Provisional application No. 63/499,765, filed on May 3, 2023.

(51) Int. Cl. C07D 471/04; A61K 31/444

(52) U.S. Cl. CPC C07D 471/04; A61K 31/444

(57) ABSTRACT

The present disclosure provides solid forms of a JAK2 inhibitor, as well as compositions thereof and methods of using and preparing the same.

### RELATED APPLICATIONS

This application claims priority to U.S. Application No. 63/499,765, filed May 3, 2023, the entire contents of which are hereby incorporated by reference.

### BACKGROUND

Janus kinase 2 (JAK2) is a non-receptor tyrosine kinase involved in the JAK-STAT signaling pathway, which plays a role in cell processes such as immunity, cell division, and cell death. Dysfunction of the JAK-STAT pathway is implicated in various diseases, including cancer and other proliferative diseases, as well as diseases of the immune system. For example, essentially all BCR-ABL1-negative myeloproliferative neoplasms are associated with mutations that activate JAK2. In particular, JAK2V617F is the most prevalent mutation in myeloproliferative neoplasms, occurring in approx. 70% of all patients, and in up to 95% of patients with polycythemia vera. (Vainchenker, W., Kralovics, R. Blood 2017, 129(6): 667-79). Even less common mutations, such as in MPL and CALR, have been shown to effect activation of JAK2, thereby initiating and/or driving disease progression. (Vainchenker, W. et al., F1000Rescarch 2018, 7(F1000 Faculty Rev): 82). Furthermore, polymorphisms in JAK2 have been linked to various autoimmune diseases and inflammatory conditions, such as psoriasis and inflammatory bowel disease. (O'Shea, J. J. et al., Ann. Rheum. Dis. 2013 April, 72: ii111-ii115). Increased signaling through JAK2, as well as other members of the JAK family, is also associated with atopic dermatitis. (Rodrigues, M. A. and Torres, T. J. Derm. Treat. 2019, 31(1): 33-40).

Inhibitors of JAKs (e.g., JAK2) are classified based on their binding mode. All currently approved JAK inhibitors are Type I inhibitors, which are those that bind the ATP-binding site in the active conformation of the kinase domain, thereby blocking catalysis (Vainchenker, W. et al.). However, increased phosphorylation of the JAK2 activation loop is observed with Type I inhibitors and may lead to acquired resistance in certain patients (Meyer S. C., Levine, R. L. Clin. Cancer Res. 2014, 20(8): 2051-9). Type II inhibitors, on the other hand, bind the ATP-binding site of the kinase domain in the inactive conformation and, therefore, may avoid hyperphosphorylation observed with Type I inhibitors (Wu, S. C. et al. Cancer Cell 2015 Jul. 13, 28(1): 29-41).

### SUMMARY

Chemical compounds can form one or more different pharmaceutically acceptable solid forms, including amorphous and polymorphic crystal forms. Individual solid forms of bioactive chemical compounds can have different properties. There is a need for the identification and selection of appropriate solid forms of bioactive chemical compounds (including appropriate crystalline forms, where applicable) for the development of pharmaceutically acceptable dosage forms for the treatment of various diseases or conditions associated with JAK2.

The present disclosure provides novel solid forms useful as inhibitors of JAK2. In general, provided forms, and pharmaceutically acceptable compositions thereof, are useful for treating or lessening the severity of a variety of diseases or disorders as described in detail herein.

### BRIEF DESCRIPTION OF THE DRAWINGS

FIG. 1 provides an overlay of XRPD spectra, from top to bottom: Compound 1 Lot I, Compound 1 Lot II, Compound 1 Form B, and Compound 1 Form C.

FIG. 2 provides TGA (top) and DSC (bottom) curves for Compound 1 Lot I.

FIG. 3A provides an overlay of XRPD spectra, from top to bottom: Compound 1 Form A, Compound 1 Form B, Compound 1 Form C, Compound 1 Form D, Compound 1 Form E, and Compound 1 Form F.

FIG. 3B provides an overlay of XRPD spectra, from top to bottom: Compound 1 Form G, Compound 1 Form H, a mixture of Compound 1 Form H and Compound 1 Form I, Compound 1 Form J, Compound 1 Form K, and Compound 1 Form L.

FIG. 3C provides an overlay of XRPD spectra, from top to bottom: Compound 1 Form M, Compound 1 Form N, Compound 1 Form O, a mixture of Compound 1 Form P and Compound 1 Form Q, and a mixture of Form S and Compound 1 Form B.

FIG. 4 provides an overlay of XRPD spectra from interconversion slurry experiments, from top to bottom: Compound 1 Form B after slurry in EtOH:H₂O (90:10) for 9 days at room temperature; Compound 1 Form B after slurry in EtOH:H₂O (90:10) for 9 days at 65° C.; and Compound 1 Form B reference spectrum.

FIG. 5 provides an XRPD spectrum of Compound 1 Form B.

FIG. 6 provides TGA (top) and DSC (bottom) curves for Compound 1 Form B.

FIG. 7 provides a DVS plot for Compound 1 Form B.

FIG. 8 provides an overlay of XRPD spectra taken before (top) and after (bottom) a DVS experiment with Compound 1 Form B.

FIG. 9 provides an overlay of XRPD spectra. Top: Compound 1 Form B after slurrying in water for 5 days at RT; bottom: Compound 1 Form B reference spectrum.

FIG. 10 provides an XRPD spectrum of Compound 1 Form C.

FIG. 11 provides TGA (top) and DSC (bottom) curves for Compound 1 Form C.

FIG. 12 provides a DVS plot for Compound 1 Form C.

FIG. 13 provides an overlay of XRPD spectra taken before (top) and after (bottom) a DVS experiment with Compound 1 Form C.

FIG. 14 provides an XRPD spectrum of Compound 1 Form D.

FIG. 15 provides TGA (top) and DSC (bottom) curves for Compound 1 Form D.

FIG. 16 provides an XRPD spectrum of Compound 1 Form E.

FIG. 17 provides an XRPD spectrum of Compound 1 Form F.

FIG. 18 provides TGA (top) and DSC (bottom) curves for Compound 1 Form F.

FIG. 19 provides a DVS plot for Compound 1 Form F.

FIG. 20 provides an overlay of XRPD spectra taken before (top) and after (bottom) a DVS experiment with Compound 1 Form F.

FIG. 21 provides an overlay of XRPD spectra taken before (top, Form F) and after (bottom, Form C) slurrying Compound 1 Form Fin water at ambient temperature for 3 days.

FIG. 22 provides an XRPD spectrum of Compound 1 Form G.

FIG. 23 provides an overlay of XRPD spectra. From top to bottom: Compound 1 Form G with a minor amount of Compound 1 Form P (Sample 3.5.4 from Table 3.5); Compound 1 Form G reference spectrum; and a mixture of Compound 1 Form P and Form Q (Sample 3.2.3, Table 3.2).

FIG. 24 provides TGA (top) and DSC (bottom) curves for Compound 1 Form G, obtained from a sample comprising Compound 1 Form G with a minor amount of Compound 1 Form P (Sample 3.5.4 from Table 3.5).

FIG. 25 provides TGA (top) and DSC (bottom) curves for Compound 1 Form H.

FIG. 26 provides an XRPD spectrum of Compound 1 Form J.

FIG. 27 provides an XRPD spectrum of Compound 1 Form K.

FIG. 28 provides an XRPD spectrum of Compound 1 Form L.

FIG. 29 provides TGA (top) and DSC (bottom) curves for Compound 1 Form L.

FIG. 30 provides an XRPD spectrum of Compound 1 Form M.

FIG. 31 provides an XRPD spectrum of Compound 1 Form N.

FIG. 32 provides an XRPD spectrum of Compound 1 Form O.

FIG. 33 is a schematic presentation of the results from certain polymorph screening experiments described herein.

FIG. 34 is scanning electron microscopy (SEM) image of amorphous Compound 1.

FIG. 35 provides an XRPD spectrum of amorphous Compound 1 (top), compared to Compound 1 Form B (bottom).

FIG. 36 provides a DSC curve of amorphous Compound 1.

### DETAILED DESCRIPTION

### Compound 1

Compound 1 is an inhibitor of JAK2 having the following structure:

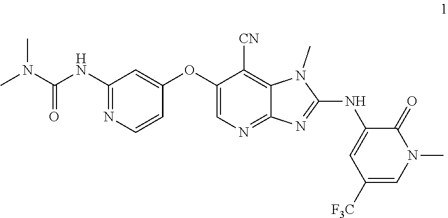

Compound 1 is also known by the following chemical name: 3-(4-((7-cyano-1-methyl-2-((1-methyl-2-oxo-5-(trifluoromethyl)-1,2-dihydropyridin-3-yl)amino)-1H-imidazo[4,5-b]pyridin-6-yl)oxy) pyridin-2-yl)-1,1-dimethylurea. The present disclosure provides various forms, including solid forms, of Compound 1, pharmaceutical compositions thereof, and methods of preparing those forms of Compound 1.

Compound 1 has demonstrated potency against JAK2 in an in vitro assay (see, e.g., Example 22 herein). Accordingly, Compound 1 is useful for treating diseases, disorder, or conditions associated with JAK2, as described herein.

### Solid Forms of Compound 1

In some embodiments, the present disclosure provides solid forms of Compound 1. In some embodiments, the present disclosure provides crystalline solid forms of Compound 1. In some embodiments, the present disclosure provides one or more polymorphic solid forms of Compound 1. As used herein, the term “polymorph” refers to the ability of a compound to exist in one or more different crystal structures. Polymorphs may vary in pharmaceutically relevant physical properties between one form and another, e.g., solubility, stability, or hygroscopicity. Solid forms (e.g., crystalline solid forms) impart or may impart characteristics such as improved aqueous solubility, stability, hygroscopicity (e.g., provided forms may be less hygroscopic than another form, e.g., an anhydrous form), absorption, bioavailability, and case of formulation.

In some embodiments, Compound 1 is provided in an amorphous form. In some embodiments, Compound 1 is provided in a crystalline form.

It will be appreciated that a crystalline solid form of Compound 1 may exist in a neat (i.e., unsolvated) form, a hydrated form, a solvated form, and/or a heterosolvated form. In some embodiments, a crystalline solid form of Compound 1 does not have any water or other solvent incorporated into the crystal lattice (i.e., is “unsolvated” or an “anhydrate”). In some embodiments, a crystalline solid form of Compound 1 comprises water and/or other solvent in the crystal lattice (i.e., are hydrates and/or solvates, respectively). It will be appreciated that solvates comprising only certain solvents (most notably, water) are suitable for development as a drug. Solvates comprising other solvents may be useful for manufacturing and/or testing, inter alia, even if they may not be acceptable for use in an approved therapeutic product.

In some embodiments, the present disclosure provides Compound 1 as a hydrate. In some embodiments, the present disclosure provides Compound 1 as an anhydrate.

In some embodiments, the present disclosure provides Compound 1 as a solvate (e.g., a solvate of acetic acid, acetone, cyclopentyl methyl ether, dichloromethane, dimethylsulfoxide, di-n-butyl ether, ethyl acetate, methyl tert-butyl ether, N-methyl-pyrrolidone, trifluoroethanol, water, or any mixture thereof).

The structure depicted for a form of Compound 1 is also meant to include all tautomeric forms of Compound 1. Additionally, structures depicted herein are meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

### Compound 1 Form A

In some embodiments, the present disclosure provides a solid form of Compound 1 referred to herein as Form A. In some embodiments, Compound 1 Form A is a hydrate.

In some embodiments, Compound 1 Form A is characterized by one or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by two or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by three or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by four or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by five or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by six or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by seven or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by eight or more peaks in its XRPD pattern selected from those at 8.53, 14.31, 15.58, 17.37, 17.72, 18.80, 20.68, 21.90, 23.34, 23.66, and 24.06 degrees 2 theta±0.20. In some embodiments, Compound 1 Form A is characterized by having peaks in its XRPD pattern at 8.53, 14.31, 15.58, 17.37, 17.72, 18.80, 20.68, 21.90, 23.34, 23.66, and 24.06 degrees 2 theta±0.20.

In some embodiments, Compound 1 Form A is characterized by an XRPD pattern having diffractions at angles (degrees 2 theta±0.20) corresponding to substantially all of:

7.38
8.53
9.40
11.05
11.99
12.42
13.48
14.31
14.98
15.58
16.03
17.37
17.72
18.39
18.80
19.42
20.68
21.90
23.34
23.66
24.06
24.99
25.53
26.02
27.12
28.81

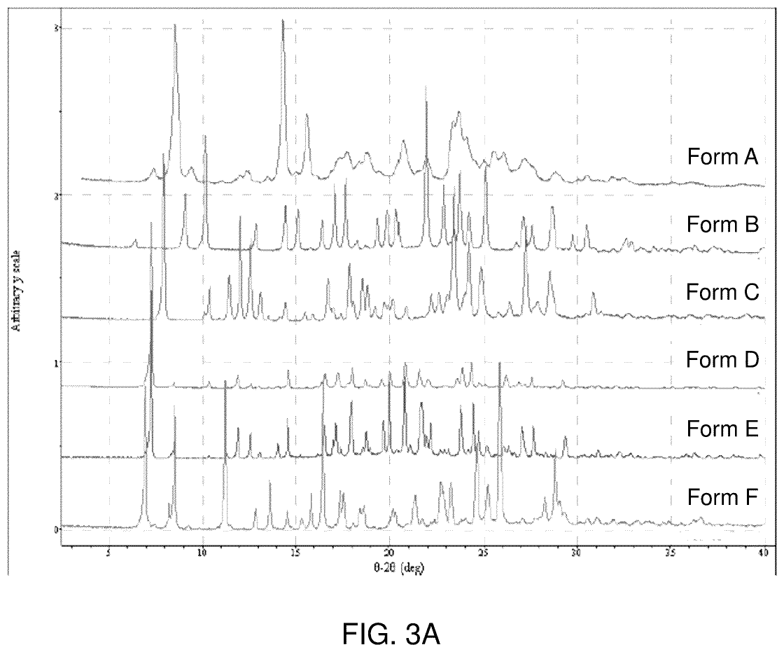

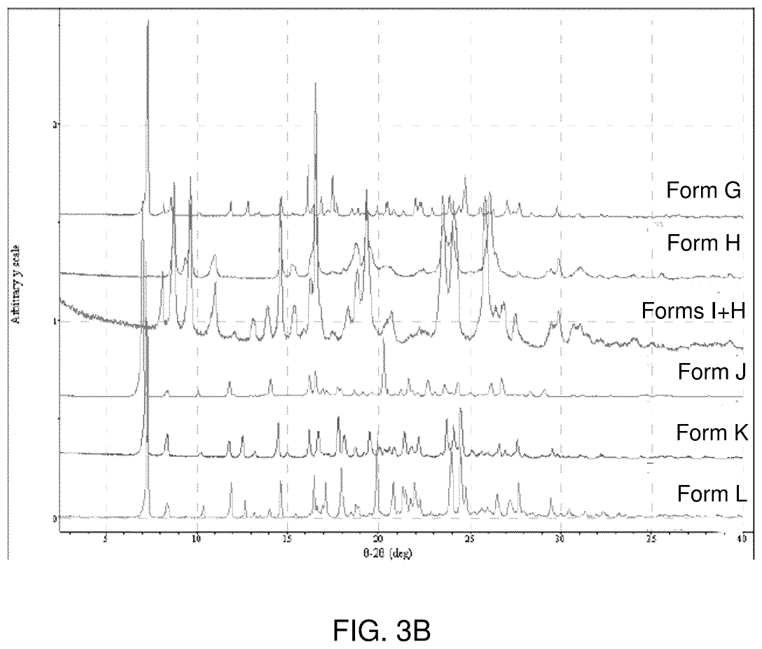

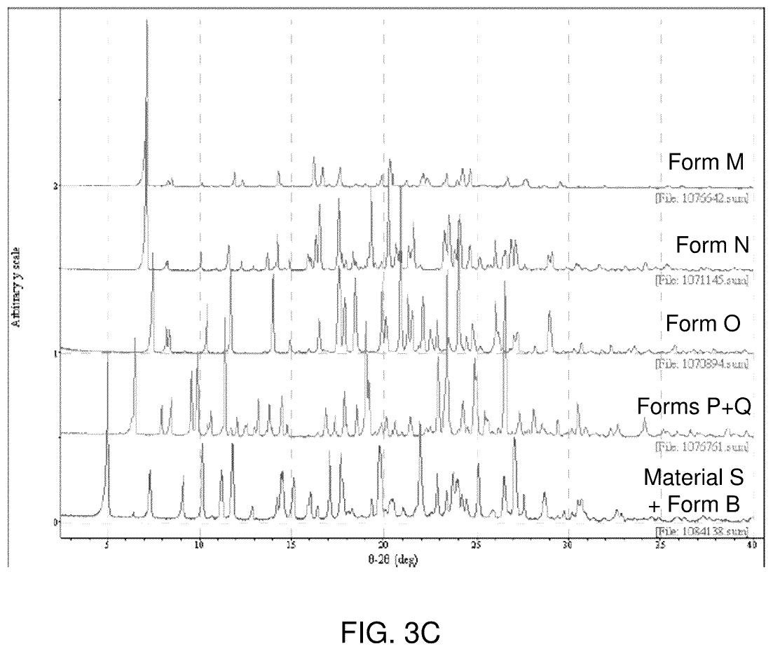

In some embodiments, Compound 1 Form A is characterized by an XRPD pattern substantially similar to that provided in FIG. 3A (top spectrum).

### Compound 1 Form B

In some embodiments, the present disclosure provides a solid form of Compound 1 referred to herein as Form B. In some embodiments, Compound 1 Form B is an anhydrate (i.e., unsolvated).

In some embodiments, Compound 1 Form B is characterized by one or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20. In some embodiments, Compound 1 Form B is characterized by two or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20. In some embodiments, Compound 1 Form B is characterized by three or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20. In some embodiments, Compound 1 Form B is characterized by four or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20. In some embodiments, Compound 1 Form B is characterized by five or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20. In some embodiments, Compound 1 Form B is characterized by six or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta #0.20. In some embodiments, Compound 1 Form B is characterized by seven or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20. In some embodiments, Compound 1 Form B is characterized by eight or more peaks in its XRPD pattern selected from those at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20. In some embodiments, Compound 1 Form B is characterized by having peaks in its XRPD pattern at 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67 degrees 2 theta±0.20.

In some embodiments, Compound 1 Form B is characterized by one or more peaks in its XRPD pattern selected from those at 9.05, 9.09, 10.16, 12.85, 15.10, and 19.35 degrees 2 theta±0.20. In some embodiments, Compound 1 Form B is characterized by two or more peaks in its XRPD pattern selected from those at 9.05, 9.09, 10.16, 12.85, 15.10, and 19.35 degrees 2 theta±0.20. In some embodiments, Compound 1 Form B is characterized by three or more peaks in its XRPD pattern selected from those at 9.05, 9.09, 10.16, 12.85, 15.10, and 19.35 degrees 2 theta±0.20. In some embodiments, Compound 1 Form B is characterized by four or more peaks in its XRPD pattern selected from those at 9.05, 9.09, 10.16, 12.85, 15.10, and 19.35 degrees 2 theta±0.20. In some embodiments, Compound 1 Form B is characterized by five or more peaks in its XRPD pattern selected from those at 9.05, 9.09, 10.16, 12.85, 15.10, and 19.35 degrees 2 theta±0.20. In some embodiments, Compound 1 Form B is characterized by having peaks in its XRPD pattern at 9.05, 9.09, 10.16, 12.85, 15.10, and 19.35 degrees 2 theta±0.20.

In some embodiments, Compound 1 Form B is characterized by an XRPD pattern having diffractions at angles (degrees 2 theta±0.20) corresponding to substantially all of:

6.40
9.05
9.09
10.16
12.85
14.42
15.10
15.79
16.39
17.08
17.66
18.28
18.72
19.35
19.85
20.35
21.89
22.83
23.37
23.71
24.20
25.09
26.73
27.10
27.57
28.59
28.67
29.06
29.37
29.75
30.49
30.85
31.92
32.59
32.87

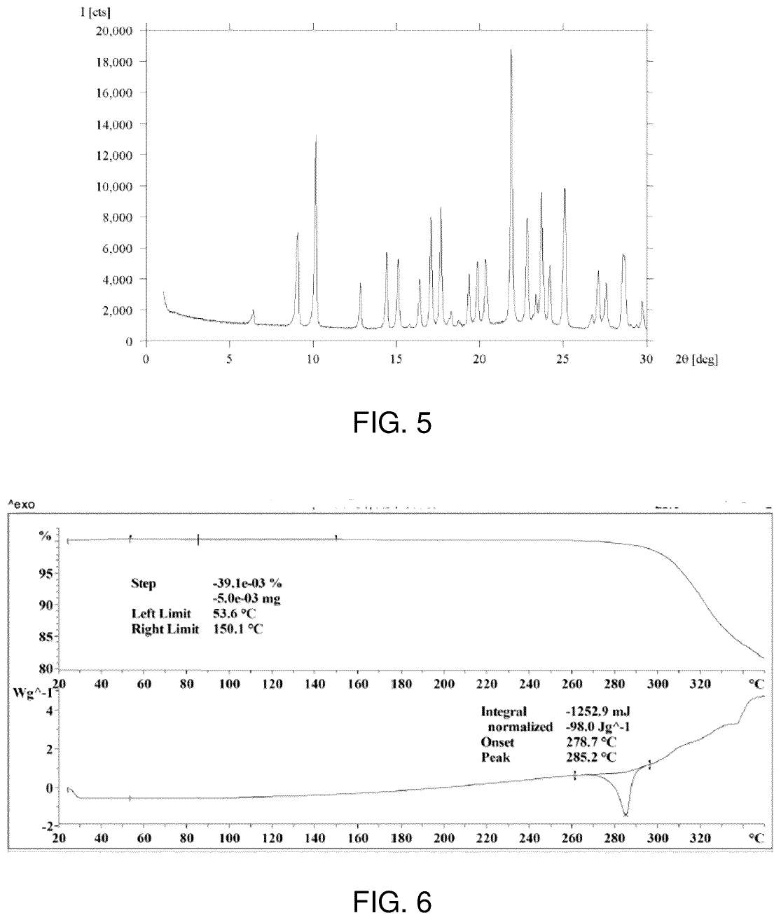

In some embodiments, Compound 1 Form B is characterized by an XRPD pattern substantially similar to that provided in FIG. 5. In some embodiments, Compound 1 Form B is characterized by a TGA pattern substantially similar to that provided in FIG. 6. In some embodiments, Compound 1 Form B is characterized by a DSC pattern substantially similar to that provided in FIG. 6.

### Compound 1 Form C

In some embodiments, the present disclosure provides a solid form of Compound 1 referred to herein as Form C. In some embodiments, Compound 1 Form C is a hydrate.

In some embodiments, Compound 1 Form C is characterized by one or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by two or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by three or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by four or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by five or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by six or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by seven or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by eight or more peaks in its XRPD pattern selected from those at 7.92, 10.36, 11.44, 12.02, 12.57, 13.10, 16.71, 17.86, 18.54, 18.82, 23.40, 24.19, 24.85, 27.23, and 28.51 degrees 2 theta±0.20. In some embodiments, Compound 1 Form C is characterized by having peaks in its XRPD pattern at 7.92, 10.36, 11.44, 12.02, 12.57, 13.10, 16.71, 17.86, 18.54, 18.82, 23.40, 24.19, 24.85, 27.23, and 28.51 degrees 2 theta±0.20.

In some embodiments, Compound 1 Form C is characterized by an XRPD pattern having diffractions at angles (degrees 2 theta±0.20) corresponding to substantially all of:

7.92
10.10
10.36
10.89
11.44
12.02
12.57
13.10
13.53
14.13
14.42
15.47
15.90
16.71
16.98
17.40
17.86
18.07
18.54
18.82
19.23
19.73
19.94
20.17
20.82
22.17
22.58
23.03
23.40
23.97
24.19
24.85
25.79
26.35
27.23

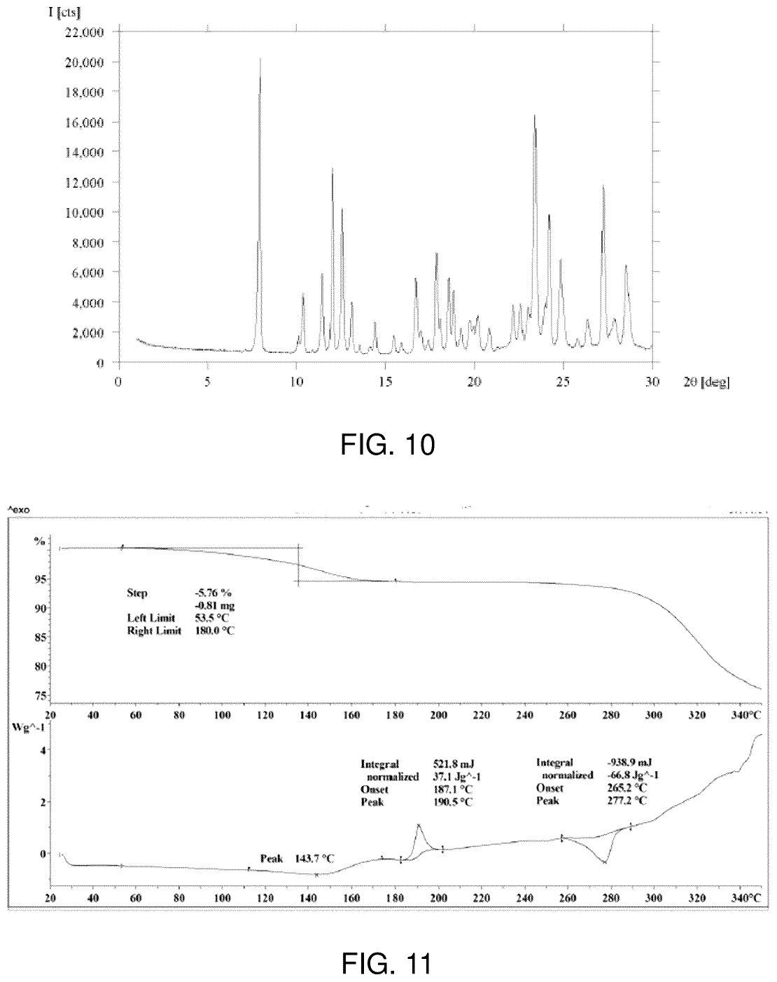

In some embodiments, Compound 1 Form C is characterized by an XRPD pattern substantially similar to that provided in FIG. 10. In some embodiments, Compound 1 Form C is characterized by a TGA pattern substantially similar to that provided in FIG. 11. In some embodiments, Compound 1 Form C is characterized by a DSC pattern substantially similar to that provided in FIG. 11.

### Compound 1 Form D

In some embodiments, the present disclosure provides a solid form of Compound 1 referred to herein as Form D. In some embodiments, Compound 1 Form D is an acetone monosolvate.

In some embodiments, Compound 1 Form D is characterized by one or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by two or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by three or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by four or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by five or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by six or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by seven or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by eight or more peaks in its XRPD pattern selected from those at 7.27, 11.87, 14.58, 16.57, 17.24, 18.01, 20.03, 20.77, 21.52, 23.85, and 24.32 degrees 2 theta±0.20. In some embodiments, Compound 1 Form D is characterized by having peaks in its XRPD pattern at 7.27, 11.87, 14.58, 16.57, 17.24, 18.01, 20.03, 20.77, 21.52, 23.85, and 24.32 degrees 2 theta±0.20.

In some embodiments, Compound 1 Form D is characterized by an XRPD pattern having diffractions at angles (degrees 2 theta±0.20) corresponding to substantially all of:

7.27
8.31
8.47
10.33
11.87
12.58
12.99
13.56
14.04
14.58
16.11
16.360
16.57
17.24
17.79
18.01
18.72
18.95
19.58
20.03
20.77
21.52
21.98
22.05
22.89
23.57
23.85
24.32
24.74
25.07
25.33
25.68
26.17
26.55
26.85

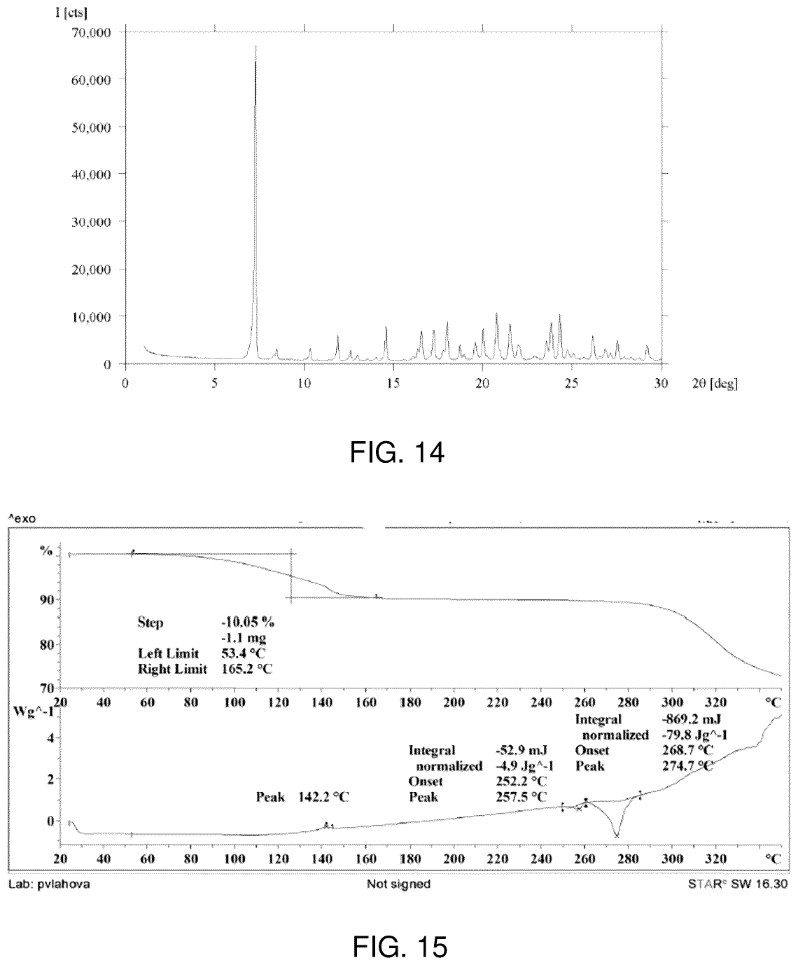

In some embodiments, Compound 1 Form D is characterized by an XRPD pattern substantially similar to that provided in FIG. 14. In some embodiments, Compound 1 Form D is characterized by a TGA pattern substantially similar to that provided in FIG. 15. In some embodiments, Compound 1 Form D is characterized by a DSC pattern substantially similar to that provided in FIG. 15.

### Compound 1 Form E

In some embodiments, the present disclosure provides a solid form of Compound 1 referred to herein as Form E. In some embodiments, Compound 1 Form E is a DMSO disolvate.

In some embodiments, Compound 1 Form E is characterized by one or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by two or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by three or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by four or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by five or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by six or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by seven or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by eight or more peaks in its XRPD pattern selected from those at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20. In some embodiments, Compound 1 Form E is characterized by having peaks in its XRPD pattern at 7.26, 11.89, 12.56, 14.56, 16.51, 17.15, 17.94, 17.98, 18.77, 19.68, 19.99, 20.75, 21.65, 22.14, 23.76, 24.42, 27.04, and 27.63 degrees 2 theta±0.20.

In some embodiments, Compound 1 Form E is characterized by an XRPD pattern having diffractions at angles (degrees 2 theta±0.20) corresponding to substantially all of:

7.26
8.47
10.32
11.32
11.89
12.56
13.06
14.03
14.56
15.23
16.16
16.51
16.99
17.15
17.40
17.94
17.98
18.28
18.57
18.77
18.95
19.68
19.99
20.39
20.75
21.05
21.65
21.92
22.14
22.66
22.89
23.10
23.76
24.16
24.42

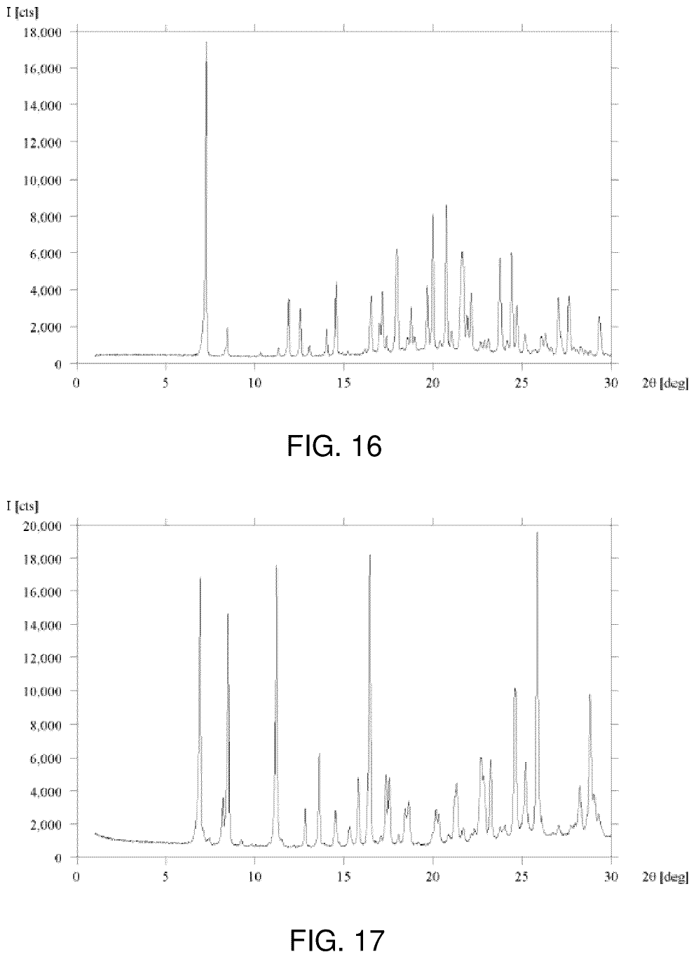

In some embodiments, Compound 1 Form E is characterized by an XRPD pattern substantially similar to that provided in FIG. 16.

### Compound 1 Form F

In some embodiments, the present disclosure provides a solid form of Compound 1 referred to herein as Form F. In some embodiments, Compound 1 Form F is an acetic acid monosolvate.

In some embodiments, Compound 1 Form F is characterized by one or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by two or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by three or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by four or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by five or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by six or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by seven or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by eight or more peaks in its XRPD pattern selected from those at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20. In some embodiments, Compound 1 Form F is characterized by having peaks in its XRPD pattern at 6.93, 8.50, 11.21, 13.60, 15.81, 16.44, 17.35, 17.54, 21.32, 22.69, 22.83, 23.23, 24.60, 25.20, 25.84, and 28.81 degrees 2 theta±0.20.

In some embodiments, Compound 1 Form F is characterized by an XRPD pattern having diffractions at angles (degrees 2 theta±0.20) corresponding to substantially all of:

6.93
8.19
8.50
9.25
11.21
12.83
13.60
14.53
15.30
15.81
16.44
17.08
17.35
17.54
18.05
18.43
18.64
20.16
20.32
20.86
21.32
21.69
22.14
22.33
22.69
22.83
23.23
23.76
24.03
24.60
25.20
25.84
27.06
27.73
28.24

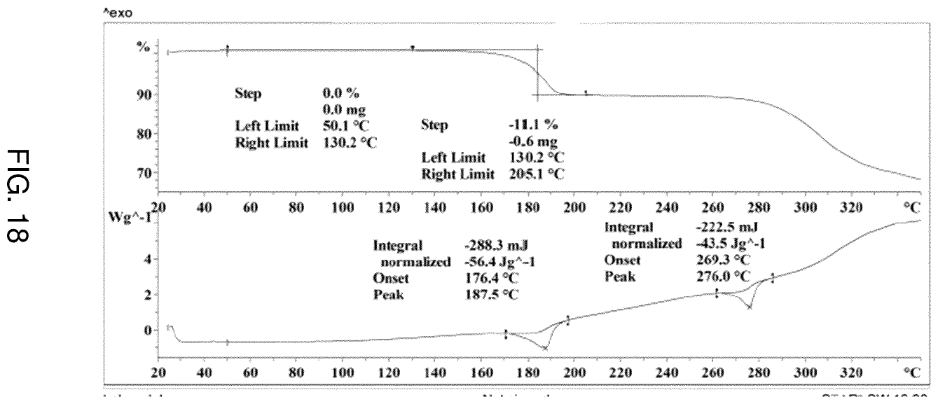

In some embodiments, Compound 1 Form F is characterized by an XRPD pattern substantially similar to that provided in FIG. 17. In some embodiments, Compound 1 Form F is characterized by a TGA pattern substantially similar to that provided in FIG. 18. In some embodiments, Compound 1 Form F is characterized by a DSC pattern substantially similar to that provided in FIG. 18.

### Compound 1 Form G

In some embodiments, the present disclosure provides a solid form of Compound 1 referred to herein as Form G. In some embodiments, Compound 1 Form G is an ethyl acetate monosolvate.

In some embodiments, Compound 1 Form G is characterized by one or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by two or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by three or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by four or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by five or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by six or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by seven or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by eight or more peaks in its XRPD pattern selected from those at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20. In some embodiments, Compound 1 Form G is characterized by having peaks in its XRPD pattern at 7.30, 8.16, 8.57, 11.86, 12.80, 14.64, 16.11, 16.85, 17.48, 17.70, 20.43, 21.97, 22.18, 22.30, 23.85, 24.72, and 27.02 degrees 2 theta±0.20.

In some embodiments, Compound 1 Form G is characterized by an XRPD pattern having diffractions at angles (degrees 2 theta±0.20) corresponding to substantially all of:

7.30
8.16
8.57
10.16
11.86
12.80
13.23
13.41
14.19
14.64
14.99
15.28
15.47
16.11
16.38
16.85
17.23
17.48
17.70
18.52
18.86
19.09
19.20
19.45
19.93
20.16
20.43
20.78
21.31
21.97
22.18
22.30
22.89
23.54
23.85

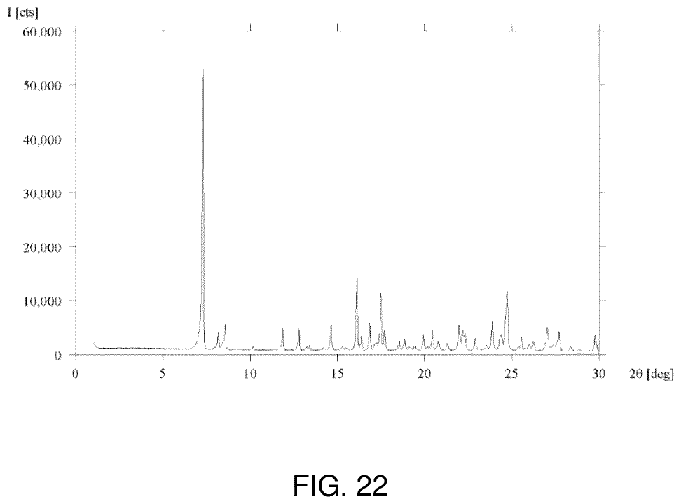

In some embodiments, Compound 1 Form G is characterized by an XRPD pattern substantially similar to that provided in FIG. 22. In some embodiments, Compound 1 Form G is characterized by a TGA pattern substantially similar to that provided in FIG. 24. In some embodiments, Compound 1 Form G is characterized by a DSC pattern substantially similar to that provided in FIG. 24.

### Compound 1 Form H

In some embodiments, the present disclosure provides a solid form of Compound 1 referred to herein as Form H. In some embodiments, Compound 1 Form H is an anhydrate (i.e., unsolvated).

In some embodiments, Compound 1 Form H is characterized by one or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by two or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by three or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by four or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by five or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by six or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by seven or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by eight or more peaks in its XRPD pattern selected from those at 8.74, 9.63, 14.60, 16.53, 18.78, 19.35, 23.50, 24.02, and 26.05 degrees 2 theta±0.20. In some embodiments, Compound 1 Form H is characterized by having peaks in its XRPD pattern at 8.74, 9.63, 14.60, 16.53, 18.78, 19.35, 23.50, 24.02, and 26.05 degrees 2 theta±0.20.

In some embodiments, Compound 1 Form H is characterized by an XRPD pattern having diffractions at angles (degrees 2 theta±0.20) corresponding to substantially all of:

8.74
9.33
9.63
10.97
14.60
15.23
16.53
17.47
18.10
18.78
19.35
20.36
22.17
22.40
23.50
24.02
26.05
26.38
27.64
29.45
29.84
31.07

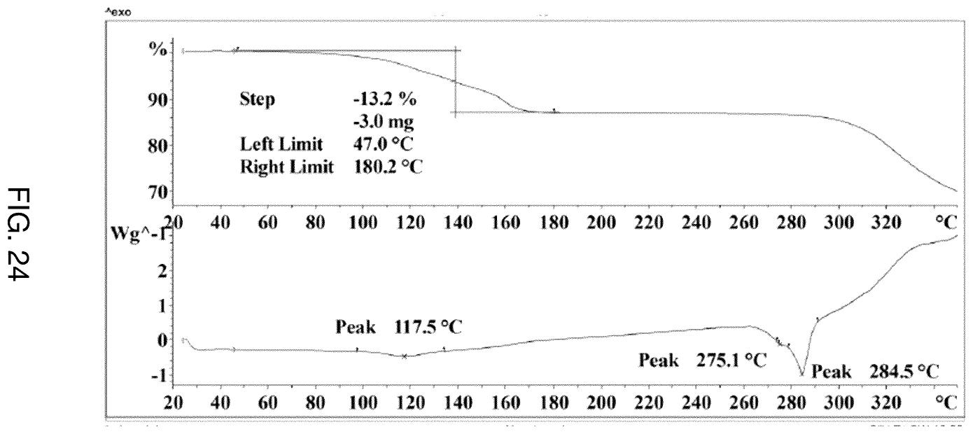

In some embodiments, Compound 1 Form H is characterized by an XRPD pattern substantially similar to that provided in FIG. 3B (second spectrum from top). In some embodiments, Compound 1 Form H is characterized by a TGA pattern substantially similar to that provided in FIG. 25. In some embodiments, Compound 1 Form H is characterized by a DSC pattern substantially similar to that provided in FIG. 25.

### Compound 1 Form I

In some embodiments, the present disclosure provides a solid form of Compound 1 referred to herein as Form I. In some embodiments, Compound 1 Form I is an anhydrate (i.e., unsolvated).

In some embodiments, Compound 1 Form I is characterized by an XRPD pattern substantially similar to that provided in FIG. 3B (third spectrum from top).

### Compound 1 Form J

In some embodiments, the present disclosure provides a solid form of Compound 1 referred to herein as Form J. In some embodiments, Compound 1 Form J is a MTBE monosolvate.

In some embodiments, Compound 1 Form J is characterized by one or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by two or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by three or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by four or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by five or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by six or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by seven or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by eight or more peaks in its XRPD pattern selected from those at 7.00, 11.79, 14.06, 16.19, 16.52, 20.28, 21.58, 22.65, and 26.73 degrees 2 theta±0.20. In some embodiments, Compound 1 Form J is characterized by having peaks in its XRPD pattern at 7.00, 11.79, 14.06, 16.19, 16.52, 20.28, 21.58, 22.65, and 26.73 degrees 2 theta±0.20.

In some embodiments, Compound 1 Form J is characterized by an XRPD pattern having diffractions at angles (degrees 2 theta±0.20) corresponding to substantially all of:

7.00
8.35
10.09
10.99
11.27
11.79
12.02
12.97
13.79
14.06
14.44
14.82
15.10
16.19
16.52
16.92
17.16
17.46
17.73
17.91
18.66
19.14
19.61
19.86
20.28
20.86
21.16
21.58
21.81
22.10
22.65
23.05
23.59
23.94
24.30
24.98
26.13
26.73
27.14
27.44
27.82
28.31
29.08

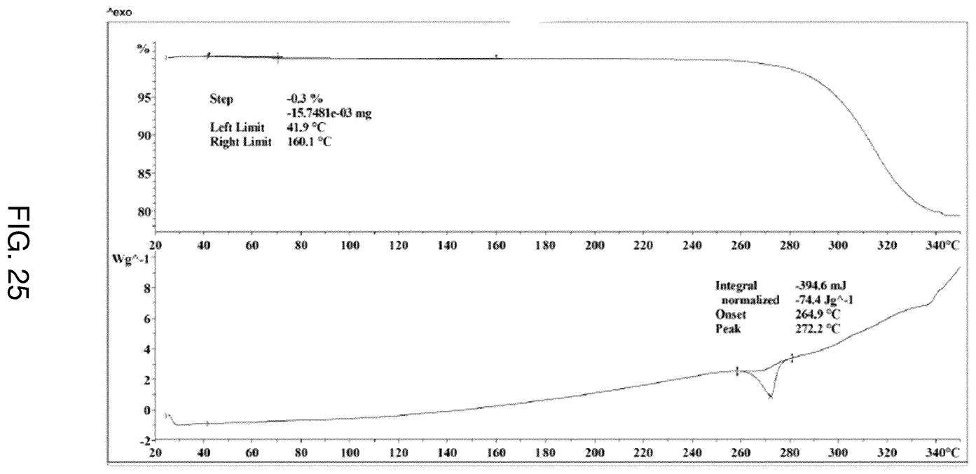

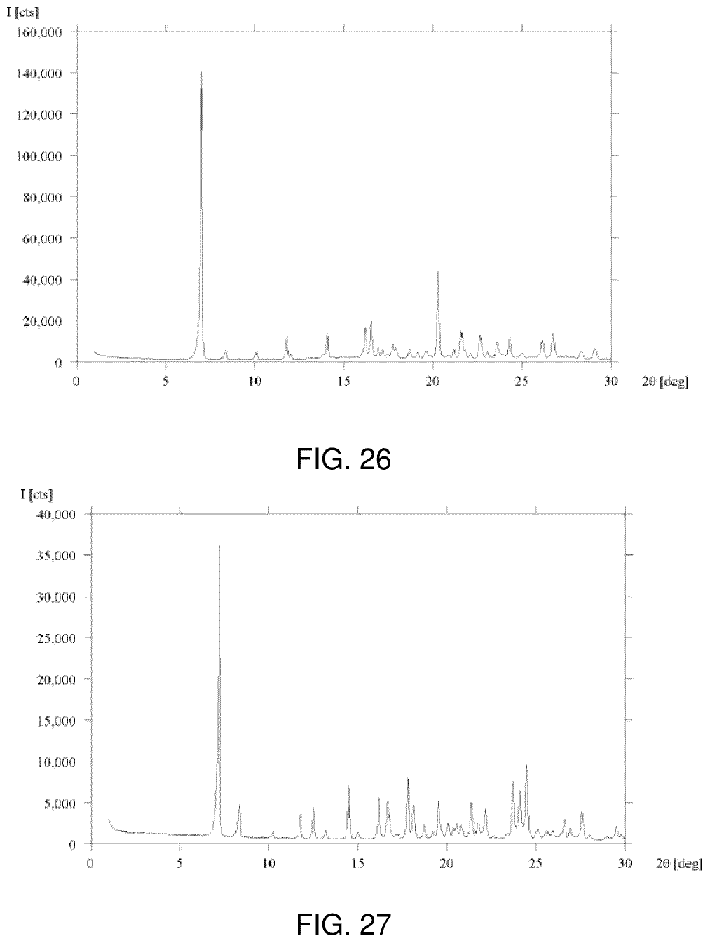

In some embodiments, Compound 1 Form J is characterized by an XRPD pattern substantially similar to that provided in FIG. 26.

### Compound 1 Form K

In some embodiments, the present disclosure provides a solid form of Compound 1 referred to herein as Form K. In some embodiments, Compound 1 Form K is a Bu₂O monosolvate.

In some embodiments, Compound 1 Form K is characterized by one or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by two or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by three or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by four or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by five or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by six or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by seven or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by eight or more peaks in its XRPD pattern selected from those at 7.21, 8.35, 11.77, 12.50, 14.47, 16.17, 16.67, 17.80, 18.13, 19.51, 21.37, 22.15, 23.70, 24.08, and 24.47 degrees 2 theta±0.20. In some embodiments, Compound 1 Form K is characterized by having peaks in its XRPD pattern at 7.21, 8.35, 11.77, 12.50, 14.47, 16.17, 16.67, 17.80, 18.13, 19.51, 21.37, 22.15, 23.70, 24.08, and 24.47 degrees 2 theta±0.20.

In some embodiments, Compound 1 Form K is characterized by an XRPD pattern having diffractions at angles (degrees 2 theta±0.20) corresponding to substantially all of:

7.21
8.35
10.22
11.77
12.50
13.18
14.47
14.98
16.17
16.67
17.13
17.25
17.80
18.13
18.73
19.20
19.51
20.07
20.35
20.55
20.79
21.37
21.73
22.15
22.62
23.38
23.70
24.08
24.47
25.09
25.61
25.93
26.58
26.91
27.58
27.99
28.98
29.50

In some embodiments, Compound 1 Form K is characterized by an XRPD pattern substantially similar to that provided in FIG. 27.

### Compound 1 Form L

In some embodiments, the present disclosure provides a solid form of Compound 1 referred to herein as Form L. In some embodiments, Compound 1 Form L is a DCM monosolvate.

In some embodiments, Compound 1 Form L is characterized by one or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by two or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by three or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by four or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by five or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by six or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by seven or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by eight or more peaks in its XRPD pattern selected from those at 7.30, 11.88, 14.62, 16.45, 17.10, 17.96, 19.92, 20.75, 21.28, 21.92, 23.96, 24.47, 24.75, and 27.65 degrees 2 theta±0.20. In some embodiments, Compound 1 Form L is characterized by having peaks in its XRPD pattern at 7.30, 11.88, 14.62, 16.45, 17.10, 17.96, 19.92, 20.75, 21.28, 21.92, 23.96, 24.47, 24.75, and 27.65 degrees 2 theta±0.20.

In some embodiments, Compound 1 Form L is characterized by an XRPD pattern having diffractions at angles (degrees 2 theta±0.20) corresponding to substantially all of:

7.30
8.34
8.43
9.34
10.32
11.88
12.65
13.14
13.44
14.01
14.62
15.43
16.21
16.45
16.61
16.93
17.10
17.96
18.49
18.77
18.89
19.54
19.92
20.75
21.03
21.28
21.42
21.70
21.92
22.21
22.82
23.96
24.47
24.75
25.21
25.64
25.93
26.10

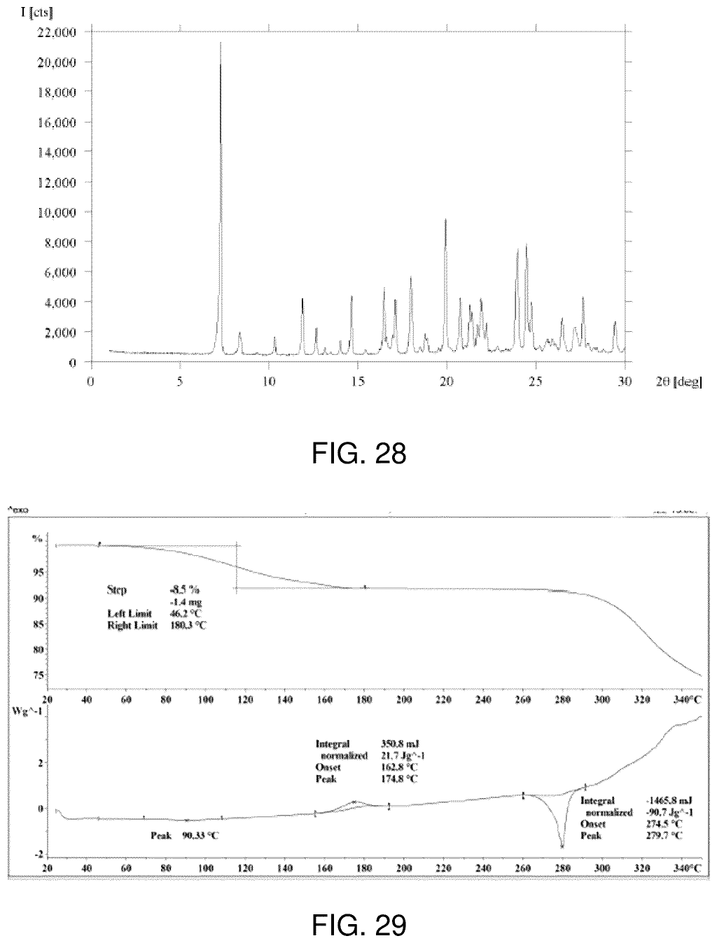

In some embodiments, Compound 1 Form L is characterized by an XRPD pattern substantially similar to that provided in FIG. 28. In some embodiments, Compound 1 Form L is characterized by a TGA pattern substantially similar to that provided in FIG. 29. In some embodiments, Compound 1 Form L is characterized by a DSC pattern substantially similar to that provided in FIG. 29.

### Compound 1 Form M

In some embodiments, the present disclosure provides a solid form of Compound 1 referred to herein as Form M. In some embodiments, Compound 1 Form M is a CPME monosolvate.

In some embodiments, Compound 1 Form M is characterized by one or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by two or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by three or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by four or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by five or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by six or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by seven or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by eight or more peaks in its XRPD pattern selected from those at 7.13, 11.89, 14.29, 16.21, 16.67, 17.63, 19.89, 20.35, 22.07, 23.36, 24.22, and 24.63 degrees 2 theta±0.20. In some embodiments, Compound 1 Form M is characterized by having peaks in its XRPD pattern at 7.13, 11.89, 14.29, 16.21, 16.67, 17.63, 19.89, 20.35, 22.07, 23.36, 24.22, and 24.63 degrees 2 theta±0.20.

In some embodiments, Compound 1 Form M is characterized by an XRPD pattern having diffractions at angles (degrees 2 theta±0.20) corresponding to substantially all of:

7.13
8.30
8.49
10.13
11.10
11.89
12.33
13.23
14.29
14.43
15.02
16.21
16.67
17.06
17.63
18.50
18.73
19.00
19.62
19.89
20.35
20.68
21.16
21.52
22.07
22.32
23.36
23.93
24.22
24.63
25.28
25.45
25.73
25.95
26.30
26.66
27.08
27.58

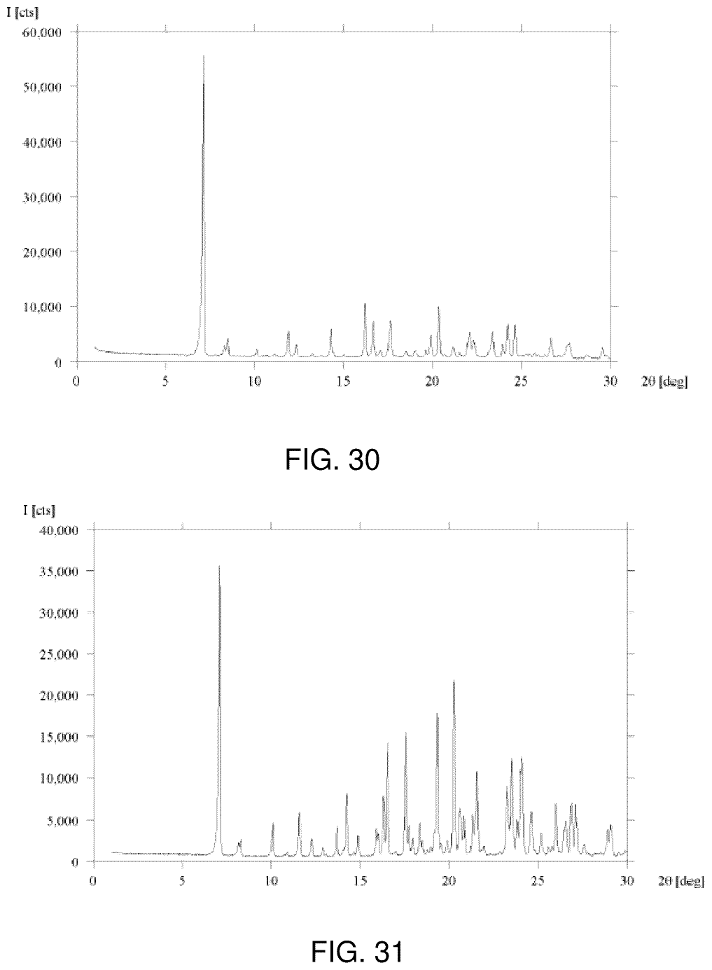

In some embodiments, Compound 1 Form M is characterized by an XRPD pattern substantially similar to that provided in FIG. 30.

### Compound 1 Form N

In some embodiments, the present disclosure provides a solid form of Compound 1 referred to herein as Form N. In some embodiments, Compound 1 Form N is an NMP solvate.

In some embodiments, Compound 1 Form N is characterized by one or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by two or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by three or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by four or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by five or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by six or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by seven or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by eight or more peaks in its XRPD pattern selected from those at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20. In some embodiments, Compound 1 Form N is characterized by having peaks in its XRPD pattern at 7.09, 11.57, 14.22, 16.31, 16.52, 17.56, 19.33, 20.24, 21.56, 23.25, 23.51, 24.01, 24.08, 25.99, 26.86, and 27.10 degrees 2 theta±0.20.

In some embodiments, Compound 1 Form N is characterized by an XRPD pattern having diffractions at angles (degrees 2 theta±0.20) corresponding to substantially all of:

7.09
8.15
8.25
10.08
10.89
11.57
12.28
12.91
13.06
13.69
14.04
14.22
14.63
14.87
15.90
16.00
16.31
16.52
16.99
17.56
17.74
17.94
18.34
18.49
18.78
18.97
19.15
19.33
19.53
19.88
20.24
20.57
20.83
21.30
21.56
21.93
22.83
23.25
23.51
23.82
24.01
24.08
24.61
24.82
25.16
25.58
25.76
25.99
26.21
26.42
26.54
26.86
27.10
27.58
27.81
28.17
28.36
28.53
28.89
29.08

In some embodiments, Compound 1 Form N is characterized by an XRPD pattern substantially similar to that provided in FIG. 31.

### Compound 1 Form O

In some embodiments, the present disclosure provides a solid form of Compound 1 referred to herein as Form O. In some embodiments, Compound 1 Form O is a TFE solvate.

In some embodiments, Compound 1 Form O is characterized by one or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by two or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by three or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by four or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by five or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by six or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by seven or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by eight or more peaks in its XRPD pattern selected from those at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20. In some embodiments, Compound 1 Form O is characterized by having peaks in its XRPD pattern at 7.44, 8.18, 8.34, 10.38, 11.70, 13.99, 16.49, 17.57, 17.87, 18.46, 19.91, 20.12, 20.85, 21.24, 21.47, 22.05, 22.83, 24.01, 26.01, and 28.96 degrees 2 theta±0.20.

In some embodiments, Compound 1 Form O is characterized by an XRPD pattern having diffractions at angles (degrees 2 theta±0.20) corresponding to substantially all of:

7.44
8.18
8.34
9.19
10.38
11.70
13.56
13.99
14.91
15.38
15.95
16.49
16.75
16.94
17.57
17.87
18.46
19.03
19.91
20.12
20.85
21.24
21.47
21.82
22.05
22.46
22.83
23.44
24.01
24.43
24.77
25.18
26.01
26.15
26.41
27.01
27.19
27.48
27.83
28.15
28.68
28.96
29.69
30.28
30.67

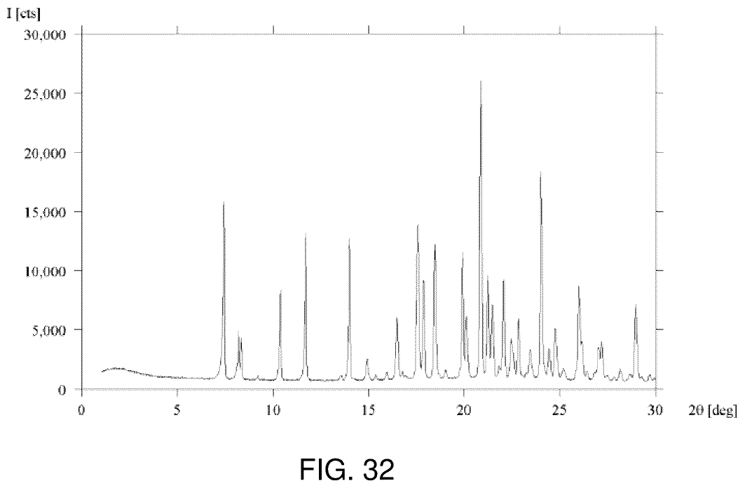

In some embodiments, Compound 1 Form O is characterized by an XRPD pattern substantially similar to that provided in FIG. 32.

### Amorphous Compound 1

In some embodiments, the present disclosure provides an amorphous form of Compound 1.

In some embodiments, an amorphous form of Compound 1 is characterized by an XRPD pattern substantially similar to that provided in FIG. 35.

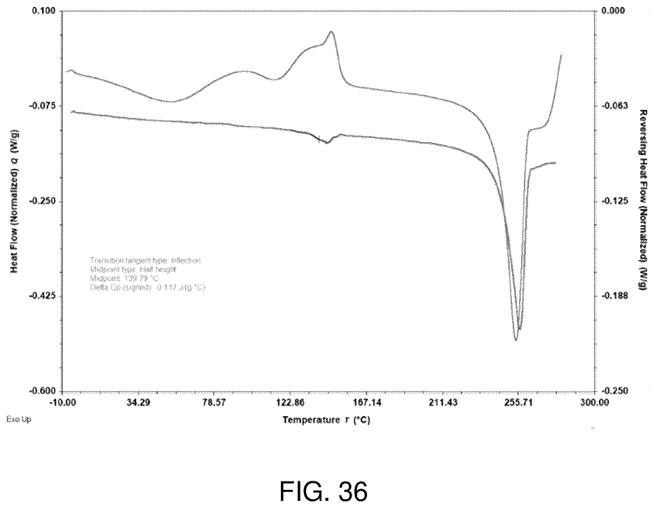

In some embodiments, an amorphous form of Compound 1 is characterized by a DSC thermogram substantially similar to that provided in FIG. 36.

In some embodiments, the present disclosure provides a composition comprising amorphous Compound 1 with a median particle size (D50) from about 25 nm to about 150 nm. In some embodiments, the present disclosure provides a composition comprising amorphous Compound 1 with a median particle size (D50) from about 25 nm to about 75 nm. In some embodiments, the present disclosure provides a composition comprising amorphous Compound 1 with a median particle size (D50) from about 25 nm to about 50 nm. In some embodiments, the present disclosure provides a composition comprising amorphous Compound 1 with a median particle size (D50) from about 25 nm to about 35 nm. In some embodiments, median particle size (D50) is measured using scanning electron microscopy, e.g., as described in Example 21.

In some embodiments, an amorphous form of Compound 1 may be prepared by such methods as lyophilization, melting, spray drying, and precipitation from supercritical fluid. In some embodiments, an amorphous form of Compound 1 is prepared as described in Example 21.

### Preparing Provided Solid Forms

In some embodiments, the present disclosure provides methods of preparing crystalline solid forms of Compound 1.

In some embodiments, a solid form of Compound 1 is prepared by combining Compound 1 (e.g., amorphous Compound 1, crystalline Compound 1, or a mixture thereof) with a suitable solvent under suitable conditions and isolating the solid form of Compound 1. In some embodiments, a solid form of Compound 1 is prepared according to a method described herein (e.g., slurry, rotary evaporation, fast evaporation, slow evaporation, crash precipitation with direct addition, crash precipitation with reverse addition, vapor diffusion, and/or heating). In some embodiments, a solid form of Compound 1 is prepared according a method described in the Examples herein.

In some embodiments, a suitable solvent is acetic acid, acetone, chloroform, cyclopentyl methyl ether, dichloromethane, dimethylacetamide, dimethylformamide, dimethylsulfoxide, di-n-butyl ether, ethanol, ethyl acetate, methanol, methyl ethyl ketone, methyl tert-butyl ether, N-methyl-pyrrolidone, tert-amyl methyl ether, trifluoroethanol, water, or any mixture thereof. In some embodiments, a suitable solvent is dimethylacetamide, dimethylformamide, dimethylsulfoxide, ethanol, water, or any mixture thereof. In some embodiments, a suitable solvent is a mixture of ethanol and water. In some embodiments, a suitable solvent is supercritical carbon dioxide.

In some embodiments, a method of preparing a solid form of Compound 1 comprises a step of heating a mixture comprising Compound 1 to a suitable temperature. In some embodiments, a method of preparing a solid form of Compound 1 comprises a step of stirring a mixture comprising Compound 1 at ambient temperature. In some embodiments, a method of preparing a solid form of Compound 1 comprises a step of cooling a mixture comprising Compound 1 to a suitable temperature.

In some embodiments, a method of preparing a solid form of Compound 1 comprises a step of dissolving Compound 1 in supercritical carbon dioxide at elevated temperature (e.g., about 60° C. to about 100° C.) and elevated pressure (e.g., about 300 bar to about 600 bar). In some such embodiments, a method further comprises a step of passing the solution through a nozzle into a collection vessel, e.g., as described in Example 21.

In some embodiments, a solid form of Compound 1 precipitates or crystallizes from a mixture (e.g., a solution, suspension, or slurry). In some embodiments, a solid form of Compound 1 precipitates or crystallizes from a mixture after cooling, addition of an anti-solvent, and/or removal of all or part of a solvent through methods such as evaporation, distillation, filtration, etc.

In some embodiments, a method of preparing a solid form of Compound 1 comprises a step of isolating the solid form. It will be appreciated that Compound 1 may be isolated by any suitable means. In some embodiments, Compound 1 is separated from a supernatant by filtration. In some embodiments, Compound 1 is separated from a supernatant by decanting.

In some embodiments, an isolated solid form of Compound 1 is dried (e.g., in air or under reduced pressure, optionally at elevated temperature).

In some embodiments, a solid form of Compound 1 is prepared by converting one solid form of Compound 1 into another solid form of Compound 1.

In some embodiments, a method of preparing a solid form of Compound 1 (e.g., Compound 1 Form B) comprises slurrying Compound 1 in a mixture of ethanol and water (e.g., a 90:10 mixture of ethanol:water). In some embodiments, a method comprises slurrying Compound 1 in a mixture of ethanol and water (e.g., a 90:10 mixture of ethanol:water) for about 10 days. In some embodiments, a method comprises slurrying Compound 1 in a mixture of ethanol and water (e.g., a 90:10 mixture of ethanol:water) at ambient temperature.

### Compositions

In some embodiments, the present disclosure provides compositions comprising one or more solid forms of Compound 1. In some embodiments, provided compositions comprise Compound 1 Form A, Compound 1 Form B, Compound 1 Form C, Compound 1 Form D, Compound 1 Form E, Compound 1 Form F, Compound 1 Form G, Compound 1 Form H, Compound 1 Form I, Compound 1 Form J, Compound 1 Form K, Compound 1 Form L, Compound 1 Form M, Compound 1 Form N, Compound 1 Form O, Compound 1 Form P, or Compound 1 Form Q, Amorphous Compound 1, or a mixture thereof.

In some embodiments, the present disclosure provides a composition comprising a solid form of Compound 1 (e.g., a solid form provided herein), wherein the composition is substantially free of impurities. As used herein, the term “substantially free of impurities” means that the composition contains no significant amount of extraneous matter. Such extraneous matter may include different forms of Compound 1, residual solvents, or any other impurities that may result from the preparation of, and/or isolation of, Compound 1. In some embodiments, at least about 95% by weight of a form of Compound 1 is present. In some embodiments, at least about 96%, about 97%, or about 98% by weight of a form of Compound 1 is present. In some embodiments, at least about 99% by weight of a form of Compound 1 is present.

The terms “about” and “approximately”, unless otherwise stated and when used herein in reference to a value, refers to a value that is similar, in context to the referenced value. In general, those skilled in the art, familiar with the context, will appreciate the relevant degree of variance encompassed by about/approximately in that context. Unless otherwise stated, in some embodiments the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would be less than 0%, or would exceed 100% of a possible value).

In some embodiments, the present disclosure provides a composition comprising a solid form of Compound 1, wherein Compound 1 is present in an amount of at least about 97.0, 97.5, 98.0, 98.5, 99.0, 99.5, or 99.8 weight percent, where the percentages are based on the total weight of the composition. In some embodiments, the present disclosure provides a composition comprising a solid form of Compound 1, wherein the composition contains no more than about 3.0 area percent HPLC of total organic impurities and, in certain embodiments, no more than about 1.5 area percent HPLC total organic impurities relative to the total area of the HPLC chromatogram. In some embodiments, a composition comprising a solid form of Compound 1 contains no more than about 1.0 area percent HPLC of any single impurity; no more than about 0.6 area percent HPLC of any single impurity, and, in certain embodiments, no more than about 0.5 area percent HPLC of any single impurity, relative to the total area of the HPLC chromatogram.

In some embodiments, the present disclosure provides a composition comprising amorphous Compound 1. In some embodiments, a composition comprising amorphous Compound 1 is substantially free of crystalline Compound 1. As used herein, the term “substantially free of crystalline Compound 1” means that the composition contains no significant amount of crystalline Compound 1. In some embodiments, at least about 95% by weight of amorphous Compound 1 is present. In some embodiments, at least about 96%, about 97%, or about 98% by weight of amorphous Compound 1 is present. In some embodiments, at least about 99% by weight of amorphous Compound 1 is present.

In some embodiments, the present disclosure provides a composition comprising crystalline Compound 1. In some embodiments, a composition comprising crystalline Compound 1 is substantially free of amorphous Compound 1. As used herein, the term “substantially free of amorphous Compound 1” means that the composition contains no significant amount of amorphous Compound 1. In some embodiments, at least about 95% by weight of crystalline Compound 1 is present. In some embodiments, at least about 96%, about 97%, or about 98% by weight of crystalline Compound 1 is present. In some embodiments, at least about 99% by weight of crystalline Compound 1 is present.

In some embodiments, the present disclosure provides a composition comprising Compound 1 in a particular crystalline form, wherein the composition is substantially free of other crystalline forms of Compound 1. For example, in some embodiments, a composition comprises Compound 1 Form B substantially free of other crystalline forms of Compound 1 (e.g., Form A, Form C, Form D, Form E, Form F, Form G, Form H, Form J, Form K, Form L, Form M, Form N, Form O, Form P and/or Form Q). As used herein, the term “substantially free of other crystalline forms of Compound 1” means that the composition contains no significant amount of other crystalline forms of Compound 1. In some embodiments, at least about 95% by weight of a particular crystalline form of Compound 1 is present. In some embodiments, at least about 96%, about 97%, or about 98% by weight of a particular crystalline form of Compound 1 is present. In some embodiments, at least about 99% by weight of a particular crystalline form of Compound 1 is present.

In some embodiments, the present disclosure provides a composition comprising at least 1 g, 2 g, 5 g, 10 g, 15 g, 20 g, 25 g, 30 g, 35 g, 40 g, or 45 g of crystalline Compound 1. In some embodiments, the present disclosure provides a composition comprising at least 1 g, 2 g, 5 g, 10 g, 15 g, 20 g, 25 g, 30 g, 35 g, 40 g, or 45 g of crystalline Compound 1, wherein the crystalline Compound 1 is substantially in a solid form selected from Form A, Form C, Form D, Form E, Form F, Form G, Form H, Form J, Form K, Form L, Form M, Form N, Form O, Form P Form Q and combination thereof. In some embodiments, the present disclosure provides a composition comprising at least 1 g, 2 g, 5 g, 10 g, 15 g, 20 g, 25 g, 30 g, 35 g, 40 g, or 45 g of crystalline Compound 1, wherein the crystalline Compound 1 is substantially in Form B. In some embodiments, the present disclosure provides a composition comprising at least 1 g Compound 1 Form B. In some embodiments, the present disclosure provides a composition comprising at least 5 g Compound 1 Form B. In some embodiments, the present disclosure provides a composition comprising at least 10 g Compound 1 Form B. In some embodiments, the present disclosure provides a composition comprising at least 20 g Compound 1 Form B. In some embodiments, the present disclosure provides a composition comprising at least 40 g Compound 1 Form B.

### Pharmaceutical Compositions

In some embodiments, the present disclosure provides pharmaceutical compositions comprising Compound 1 in any solid form described herein in combination with a pharmaceutically acceptable excipient (e.g., a carrier). In some embodiments, provided pharmaceutical compositions are solid compositions. In some embodiments, provided pharmaceutical compositions are for oral administration.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water and one or more fillers, disintegrants, lubricants, glidants, anti-adherents, and/or anti-statics, etc. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure.

Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions. Methods of preparing pharmaceutical compositions are well known in the art.

In some embodiments, provided compounds are formulated in a unit dosage form for case of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein.

### Uses

The present disclosure provides uses for solid forms and compositions described herein. In some embodiments, provided solid forms and compositions are useful in medicine (e.g., as therapy). In some embodiments, provided solid forms and compositions are useful in research as, for example, analytical tools and/or control compounds in biological assays.

In some embodiments, the present disclosure provides methods of administering provided solid forms or compositions to a subject in need thereof. In some embodiments, the present disclosure provides methods of administering provided solid forms or compositions to a subject suffering from or susceptible to a disease, disorder, or condition associated with JAK2.

In some embodiments, Compound 1 and solid forms thereof are useful as JAK2 inhibitors. In some embodiments, Compound 1 and solid forms thereof are useful as Type II JAK2 inhibitors. In some embodiments, the present disclosure provides methods of inhibiting JAK2 in a subject comprising administering a provided solid form or composition. In some embodiments, the present disclosure provides methods of inhibiting JAK2 in a biological sample comprising contacting the sample with a provided solid form or composition.

JAK (e.g., JAK2) has been implicated in various diseases, disorders, and conditions, such as myeloproliferative neoplasms (Vainchenker, W. et al., F1000Research 2018, 7(F1000 Faculty Rev): 82), atopic dermatitis (Rodrigues, M. A. and Torres, T. J. Derm. Treat. 2019, 31(1), 33-40) and acute respiratory syndrome, hyperinflammation, and/or cytokine storm syndrome (The Lancet. doi: 10.1016/S0140-6736(20) 30628-0). Accordingly, in some embodiments, the present disclosure provides methods of treating a disease, disorder or condition associated with JAK2 in a subject in need thereof comprising administering to the subject a provided solid form or composition. In some embodiments, a disease, disorder or condition is associated with overexpression of JAK2.

In some embodiments, the present disclosure provides methods of treating cancer, comprising administering a provided solid form or composition to a subject in need thereof. In some embodiments, the present disclosure provides methods of treating proliferative diseases, comprising administering a provided solid form or composition to a subject in need thereof.

In some embodiments, the present disclosure provides methods of treating a hematological malignancy, comprising administering a provided solid form or composition to a subject in need thereof. In some embodiments, a hematological malignancy is leukemia (e.g., chronic lymphocytic leukemia, acute lymphoblastic leukemia, T-cell acute lymphoblastic leukemia, chronic myelogenous leukemia, acute myelogenous leukemia, or acute monocytic leukemia). In some embodiments, a hematological malignancy is lymphoma (e.g., Burkitt's lymphoma, Hodgkin's lymphoma, or non-Hodgkin's lymphoma). In some embodiments, a non-Hodgkin's lymphoma is a B-cell lymphoma. In some embodiments, a non-Hodgkin's lymphoma is a NK/T-cell lymphoma (e.g., cutaneous T-cell lymphoma). In some embodiments, a hematological malignancy is myeloma (e.g., multiple myeloma). In some embodiments, a hematological malignancy is myeloproliferative neoplasm (e.g., polycythemia vera, essential thrombocytopenia, or myelofibrosis). In some embodiments, a hematological malignancy is myelodysplastic syndrome.

In some embodiments, the present disclosure provides methods of treating an inflammatory disease, disorder, or condition (e.g., acute respiratory syndrome, hyperinflammation, and/or cytokine storm syndrome (including those associated with COVID-19) or atopic dermatitis), comprising administering a provided solid form or composition to a subject in need thereof.

In some embodiments, a provided solid form or composition is administered as part of a combination therapy. As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

For example, in some embodiments, a provided solid form or composition is administered to a subject who is receiving or has received one or more additional therapies (e.g., an anti-cancer therapy and/or therapy to address one or more side effects of such anti-cancer therapy, or otherwise to provide palliative care). Exemplary additional therapies include BCL2 inhibitors (e.g., venetoclax), HDAC inhibitors (e.g., vorinostat), BET inhibitors (e.g., mivebresib), proteasome inhibitors (e.g., bortezomib), LSD1 inhibitors (e.g., IMG-7289), and CXCR2 inhibitors. Useful combinations of a JAK2 inhibitor with BCL2, HDAC, BET, and proteasome inhibitors have been demonstrated in cells derived from cutaneous T-cell lymphoma patients (Yumeen, S., et al., Blood Adv. 2020, 4(10), 2213-2226). A combination of a JAK2 inhibitor with a LSD1 inhibitor demonstrated good efficacy in a mouse model of myeloproliferative neoplasms (Jutzi, J. S., et al., HemaSphere 2018, 2(3), http://dx.doi.org/10.1097/HS9.0000000000000054). CXCR2 activity has been shown to modulate signaling pathways involved in tumor growth, angiogenesis, and/or metastasis, including the JAK-STAT3 pathway (Jaffer, T., Ma, D. Transl. Cancer Res. 2016, 5(Suppl. 4), S616-S628).

### EXAMPLES

The examples below are meant to illustrate certain embodiments of the disclosure, and not to limit the scope of the disclosure.

### Abbreviations

ACN Acetonitrile
B/B Birefringence and extinction
BR Birefringence
CP Crash precipitation
CPME Cyclopentylmethyl ether
DCM Dichloromethane
DMA Dimethylacetamide
DMF Dimethylformamide
DMSO Dimethylsulfoxide
DSC Differential scanning calorimetry
DVS Dynamic (water) vapor sorption
Endo Endotherm or endothermic
Exo Exotherm or exothermic
FE Fast evaporation
IPA Isopropyl alcohol
MEK Methyl ethyl ketone (2-butanone)
MIBK Methyl isobutyl ketone
MTBE Methyl tert-butyl ether
NMP N-methyl-2-pyrrolidone
NMR Nuclear magnetic resonance spectroscopy
Obs Observation
PO Preferred orientation
ppt Precipitate or precipitation
RH Relative humidity
RT Room temperature
SE Slow evaporation
slr Slurry
Soln Solution
TAME tert-Amyl methyl ether
TFE 2,2,2-trifluoroethanol
TG/TGA Thermogravimetric analysis
THF Tetrahydrofuran
vac Vacuum
VD Vapor diffusion
XRPD X-ray powder diffraction

### Instruments and Methods

### A. X-Ray Powder Diffraction (XRPD)

### PANalytical X'Pert PRO MPD Diffractometer-Transmission Geometry

Selected XRPD patterns were collected with a PANalytical X'Pert PRO MPD diffractometer using an incident beam of Cu radiation produced by an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the Si 111 peak position. A specimen of the sample was sandwiched between 3 μm thick films and analyzed in transmission geometry. A beam-stop and short antiscatter extension were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v.5.5.

### PANalytical X'Pert PRO MPD Diffractometer—Reflection Geometry

A XRPD pattern was collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Data were collected and analyzed using Data Collector software v. 2.2b. Prior to the analysis, a silicon specimen (NIST SRM 640f) was analyzed to verify the observed position of the Si 111 peak is consistent with the NIST-certified position. A specimen of the sample was packing in a nickel-coated copper well. Antiscatter slits (SS) were used to minimize the background generated by air. Soller slits for the incident and diffracted beams were used to minimize broadening from axial divergence. A diffraction pattern was collected using a scanning position-sensitive detector (X'Celerator) located 240 mm from the sample and Data Collector software v. 2.2b. The data acquisition parameters for the pattern are displayed above the image in the Data section of this report including the divergence slit (DS) and the incident-beam antiscatter slit (SS).

### PANalytical Empyrean Diffractometer—Transmission Geometry

XRPD patterns were collected with a PANalytical Empyrean diffractometer using an incident beam of Cu radiation produced using a long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 5.5.

Figures containing XRPD patterns were generated using validated software PatternMatch v.2.3.6.

The following table summarizes collection parameters for certain figures herein:

TABLE 1.1

| | X-ray | | Step | | Scan | | |
|---|---|---|---|---|---|---|---|
| | wavelength | Scan range | size | Time/step | speed | Collection | |
| FIG. | (Å) | (° 2θ) | (° 2θ) | (s/step) | (°/min) | time (s) | Instrument |
| 1 (top spectrum) | 1.5405929 | 1.0000-40.0041 | 0.0167 | 36.830 | — | — | Empyrean |
| 1 (2^{nd} spectrum from top) | 1.5405929 | 1.0000-40.0041 | 0.0167 | 36.830 | — | — | Empyrean |
| 1 (3^{rd} spectrum from top); | 1.54059 | 1.00-39.99 | 0.017 | — | 3.3 | 719 | X'Pert Pro |
| 3A (2^{nd} spectrum from top); | | | | | | | |
| and 5 | | | | | | | |
| 1 (bottom spectrum); | 1.5405929 | 1.0000-40.0041 | 0.0167 | 36.830 | — | — | Empyrean |
| 3A (3^{rd} spectrum from top); | | | | | | | |
| and 10 | | | | | | | |
| 3A (top spectrum) | 1.54059 | 3.51-39.99 | 0.017 | — | 1.2 | 1827 | X'Pert Pro |
| 3A (4^{th} spectrum from top); | 1.54059 | 1.00-39.99 | 0.017 | — | 3.2 | 720 | X'Pert Pro |
| and 14 | | | | | | | |
| 3A (5^{th} spectrum from top); | 1.5405929 | 1.0000-40.0041 | 0.0167 | 36.830 | — | — | Empyrean |
| and 16 | | | | | | | |
| 3A (bottom spectrum); and 17 | 1.5405929 | 1.0000-40.0041 | 0.0167 | 36.830 | — | — | Empyrean |
| 3B (top spectrum); and 22 | 1.54059 | 1.00-39.99 | 0.017 | — | 3.3 | 719 | X'Pert Pro |
| 3B (2^{nd} spectrum from top) | 1.5405929 | 1.0000-40.0041 | 0.0167 | 36.830 | — | — | Empyrean |
| 3B (3^{rd} spectrum from top) | 1.54059 | 1.00-39.99 | 0.017 | — | 3.2 | 723 | X'Pert Pro |
| 3B (4^{th} spectrum from top); | 1.54059 | 1.00-39.99 | 0.017 | — | 3.2 | 720 | X'Pert Pro |
| and 26 | | | | | | | |
| 3B (5^{th} spectrum from top); | 1.54059 | 1.00-39.99 | 0.017 | — | 3.3 | 719 | X'Pert Pro |
| and 27 | | | | | | | |
| 3B (bottom spectrum); and 28 | 1.5405929 | 1.0000-40.0041 | 0.0167 | 36.830 | — | — | Empyrean |
| 3C (top spectrum); and 30 | 1.54059 | 1.00-39.99 | 0.017 | — | 3.3 | 719 | X'Pert Pro |
| 3C (2^{nd} spectrum from top); | 1.5405929 | 1.0000-40.0041 | 0.0167 | 36.830 | — | — | Empyrean |
| and 31 | | | | | | | |
| 3C (3^{rd} spectrum from top); | 1.5405929 | 1.0000-40.0041 | 0.0167 | 36.830 | — | — | Empyrean |
| and 32 | | | | | | | |
| 3C (4^{th} spectrum from top) | 1.54059 | 1.00-39.99 | 0.017 | — | 3.2 | 720 | X'Pert Pro |
| 3C (bottom spectrum) | 1.54059 | 1.00-39.99 | 0.017 | — | 3.3 | 719 | X'Pert Pro |

### B. Thermogravimetric Analysis (TGA) and Differential Scanning Calorimetry (DSC)

DSC/TGA analysis was performed using a Mettler-Toledo TGA/DSC3+ analyzer. Temperature and enthalpy adjustments were performed using indium, tin, and zinc, as well as phenyl salicylate, aluminum, and gold, and then verified with indium. The balance was verified with calcium oxalate. The sample was placed in an aluminum pan. The sample was sealed, the lid pierced, then inserted into the TG furnace. The furnace was heated under nitrogen with flow of 50 mL/min. The sample was heated from 25° C. to 350° C. with heating rate of 10° C./min.

### C. Dynamic Vapor Sorption (DVS)—Intrinsic Instrument

Moisture sorption/desorption data were collected on a Surface Measurement System DVS Intrinsic instrument. Samples were not dried prior to analysis. Sorption and desorption data were collected over a range from 5% to 95% RH to 5% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### Example 1. Preparation of Compound 1

### Synthesis of Compound 1

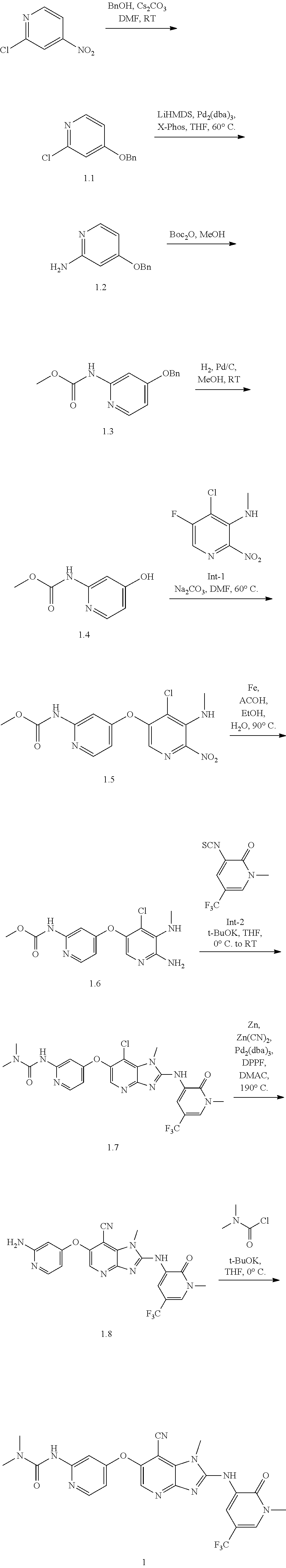

Synthesis of compound 1.1. A mixture of benzyl alcohol (102.3 g, 946.13 mmol, 1.0 equiv) and cesium carbonate (768.7 g, 2365.3 mmol, 2.5 equiv) in DMF (1000 mL) was stirred at room temperature for 2 h. A solution of 2-chloro-4-nitropyridine (150 g, 946.13 mmol, 1.0 equiv) in DMF (500 mL) was added and stirred for 16 h. It was poured into ice-water, stirred, and precipitated solids were collected by filtration and dried under vacuum to afford 1.1. MS(ES): m/z 220.5 [M+H]⁺.

Synthesis of compound 1.2. A solution of 1.1 (150 g, 682.85 mmol, 1.0 equiv) in THF (1500 mL) was degassed by bubbling through a stream of argon for 10 min. To the solution was added 2-dicyclohexyl[2′,4′,6′-tris(propan-2-yl) [1,1′-biphenyl]-2-yl]phosphane (32.55 g, 68.28 mmol, 0.1 equiv) and tris(dibenzylideneacetone) dipalladium (0) (31.26 g, 34.14 mmol, 0.05 equiv) and degassed for another 10 min. Lithium bis(trimethylsilyl)amide solution (1 M in THF, 1365 mL, 1365.7 mmol, 2.0 equiv) was added and the reaction mixture was stirred at 60° C. for 1 h. It was concentrated under reduced pressure. The residue was added to ice and 6 N hydrochloric acid (1500 mL) slowly and extracted with ethyl acetate. The aqueous layer was separated and neutralized with solid sodium bicarbonate and extracted with DCM. The combined organic layers were washed with brine, dried over anhydrous sodium sulfate, filtered, and concentrated under reduced pressure to afford 1.2. MS(ES): m/z 201.2 [M+H]⁺. It was used in the next step without purification.

Synthesis of compound 1.3. To a solution of 1.2 (100 g, 499 mmol, 1.0 equiv) in methanol (1000 mL) was added di-tert-butyl dicarbonate (130.5 g, 598.8 mmol, 1.2 equiv) at 0° C. The reaction mixture was stirred at room temperature for 3 h. After completion of reaction, precipitated solid was filtered out and rinsed with methanol, dried under vacuum to afford 1.3. MS(ES): m/z 259.2 [M+H]⁺.

Synthesis of compound 1.4. A mixture of 1.3 (106 g, 410.4 mmol, 1.0 equiv) and 10% palladium on carbon (100 g) in methanol (1000 mL) was stirred under hydrogen (1 atm) for 1 h. It was filtered through a pad of Celite® and rinsed with methanol. The filtrate was concentrated under reduced pressure to afford 1.4. MS(ES): m/z 169.1 [M+H]⁺.

Synthesis of compound 1.5. To a solution of 1.4 (66 g, 392.5 mmol, 1.0 equiv) in DMF (660 mL) was added Int-1 (64.55 g, 314 mmol, 0.8 equiv) followed by sodium carbonate (124.8 g, 1177.5 mmol, 3.0 equiv). A synthesis of Int-1 is described in WO2021/226261. The reaction mixture was stirred at 60° C. for 3 h. It was poured into ice-water, and precipitated solids were collected by filtration, dried under vacuum to afford 1.5. MS(ES): m/z 354.5 [M+H]⁺.

Synthesis of compound 1.6. To a solution of 1.5 (56 g, 158.3 mmol, 1.0 equiv) in ethanol (800 mL) and water (300 mL) was added iron powder (44.3 g, 791.5 mmol, 5.0 equiv) followed by acetic acid (47.49 g, 791.5 mmol, 5.0 equiv). The reaction mixture was heated at 90° C. for 2 h. It was cooled to room temperature and filtered through a pad of Celite®. The filtrate was concentrated under reduced pressure. The residue was purified by column chromatography on silica gel (7.0% methanol in DCM) to afford 1.6. MS(ES): m/z 324.5 [M+H]⁺.

Synthesis of compound 1.7. To a solution of 1.6 (38 g, 117.38 mmol, 1.0 equiv) and Int-2 (41.23 g, 176 mmol, 1.5 equiv) in THF (1300 mL) was added potassium tert-butoxide (1 M in THF, 704 mL, 704.28 mmol, 6.0 equiv) at 0° C. A synthesis of Int-2 is described in WO2021/226261. The reaction mixture was stirred at room temperature for 1 h. It was poured into ice-water, and product extracted with ethyl acetate. The combined organic layers were washed with brine, dried over anhydrous sodium sulfate, filtered, and concentrated under reduced pressure. The residue was purified by flash column chromatography on silica gel (CombiFlash®, 12% methanol in DCM) to afford 1.7. MS(ES): m/z: 524.2 [M+H]⁺.

Synthesis of compound 1.8. To a solution of 1.7 (0.500 g, 0.954 mmol, 1.0 equiv) in DMA (11 mL) were added zinc (0.012 g, 0.190 mmol, 0.2 equiv) and zinc cyanide (0.056 g, 0.477 mmol, 0.5 equiv). The reaction mixture was degassed by bubbling through a stream of argon for 10 min. Tris(dibenzylideneacetone) dipalladium (0) (0.131 g, 0.143 mmol, 0.15 equiv) and 1,1′-bis(diphenylphosphino) ferrocene (0.158 g, 0.286 mmol, 0.3 equiv) were added, and degassed for 5 min. The reaction mixture was stirred at 190° C. in a microwave reactor for 2 h. It was cooled to room temperature, transferred into water, and extracted with ethyl acetate. The combined organic layers were washed with brine, dried over anhydrous sodium sulfate, filtered, and concentrated under reduced pressure to afford material.

Synthesis of Compound 1. To a solution of 1.8 (0.040 g, 0.087 mmol, 1.0 equiv) and dimethylcarbamic chloride (0.010 g, 0.096 mmol, 1.1 equiv) in THF (2 mL) was added potassium tert-butoxide (1M in THF) (0.52 mL, 0.522 mmol, 6.0 equiv) at 0° C. and stirred at same temperature for 15 min. The reaction mixture was poured into ice-water, and product extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.5% methanol in DCM) to afford Compound 1. MS(ES): m/z: 528.3 [M+H]⁺, ¹H NMR (DMSO-d₆, 400 MHz): δ 9.04-9.03 (d, J=6.8 Hz 2H), 8.66 (s, 1H), 8.31 (s, 1H), 8.19 (s, 1H), 8.17 (s, 1H), 7.48 (s, 1H), 6.69 (bs, 1H), 3.97 (s, 3H), 3.67 (s, 3H), 2.90 (s, 6H).

### Preparation of Lot I

Lot I was prepared similarly to the procedure above, with the last step as follows: To a solution of 1.8 (18.6 g, 40.7 mmol, 1.0 equiv) in tetrahydrofuran (380 mL) was added dimethylcarbamic chloride (10.9 g, 101.9 mmol, 2.5 equiv), followed by addition of potassium tert-butoxide (1M in tetrahydrofuran) (244 mL, 244 mmol, 6 equiv) at 0° C. and stirred at the same temperature for 15 min. After completion of reaction, reaction mixture was transferred into ice-cold water and product was extracted with ethyl acetate (1000 mL×2). Organic layers were combined, washed with brine solution, dried over sodium sulfate and concentrated under reduced pressure at 45° C. to obtain crude material. The crude material was purified by column chromatography using methanol in dichloromethane as mobile phase. Product was eluted at 2.5% to 3.0% methanol in dichloromethane to give 12.3 g solid material. The obtained solid material was stirred in methanol (100 mL) at room temperature for 30 min. After 30 min stirring, solid was filtered and the obtained solid (11.01 g) was stirred in ethyl acetate (100 mL) at room temperature for 30 min. After 30 min stirring, solid was filtered and washed with diethyl ether (50 mL) and pentane (50 mL). The solid was dried under reduced pressure at 65° C. for 2 h to give 10.05 g Compound 1 (Yield: 46.75%)

### Preparation of Lot II

Lot II was prepared similarly to the procedure above, with the last step as follows: To a solution of 1.8 (80 g, 175.0 mmol, 1.0 equiv) in tetrahydrofuran (1600 mL) was added dimethylcarbamic chloride (28.2 g, 282.5 mmol, 1.5 equiv), followed by addition of potassium ter-butoxide (1M in tetrahydrofuran) (796 mL, 796.0 mmol, 4.55 equiv) at 0° C. and stirred at same temperature for 15 min. After completion of reaction, reaction mixture was transferred into ice-cold water and product was extracted with ethyl acetate (500 mL×2). Organic layers were combined, washed with brine solution, dried over sodium sulfate and concentrated under reduced pressure at 45° C. to obtain crude material. The crude material was purified by column chromatography using methanol in dichloromethane as mobile phase. Product was eluted at 2.5% to 3.0% methanol in dichloromethane to give 46.9 g solid material. The obtained solid was stirred in methanol (100 mL) at room temperature for 30 min. After 30 min stirring, solid was filtered and the obtained solid (51 g) was stirred in ethyl acetate (100 mL) at room temperature for 30 min. After 30 min stirring, solid was filtered and washed with diethyl ether (50 mL) and pentane (50 mL). The solid was dried under reduced pressure at 65° C. for 2 h to give 46.3 g Compound 1 (Yield: 51.93%).

### Example 2. Characterization of Compound 1 (Lots I and II)

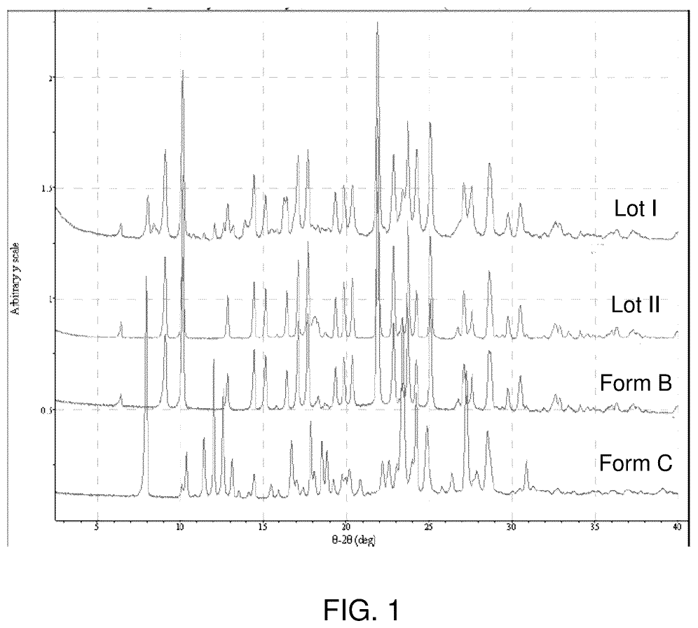

Lots I and II of Compound 1 were used as starting materials in the following examples. The XRPD patterns of Lots I and II exhibited well-resolved peaks, characteristic of crystalline material (FIG. 1). The XRPD patterns of Lots I and II failed to be indexed, suggesting that the materials were not composed of a single crystalline phase and most likely represent a mixture of crystalline phases. As described further below, the XRPD pattern of Lot I was found to be consistent with a mixture of Form B and Form C. Additional unidentified peaks were also present in the XRPD pattern of Lot I. The XRPD pattern of Lot II was consistent with Form B with a few small, unidentified peaks.

The peak positions and integration values in the proton NMR spectrum of Lot I were consistent with the chemical structure of Compound 1. The presence of ethyl acetate (0.03 mole, 0.5%) was observed, based on the presence of a triplet at 1.18 ppm and a singlet at 1.99 ppm. Traces of DCM, methanol, and some unidentified impurities were also present in the spectrum.

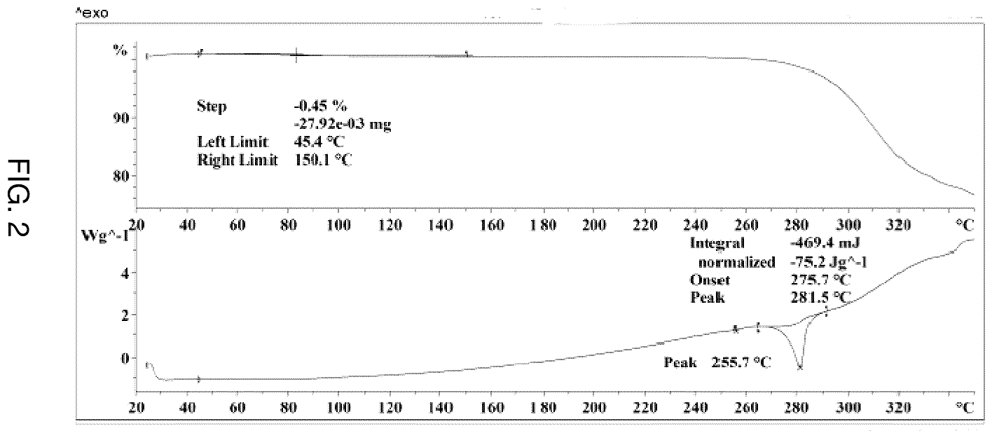

The DSC thermogram of Lot I displayed a small endotherm with peak maximum at 255.7° C., followed by a large endotherm with peak maximum at 281.5° C. and onset at 275.7° C. (FIG. 2). The TGA curve demonstrated 0.45% weight loss between 45° C. and 150° C., most likely associated with the release of residual solvents.

### Approximate Solubility Estimates

Approximate solubility estimates of Compound 1 Lot I were ascertained using the solvent incremental addition method and visual observation (Table 2.1). Aliquots of various solvents or solvent mixtures were added to measured amounts of Compound 1 Lot I with agitation at ambient temperature. Approximate solubilities were calculated based on the total solvent used to achieve a solution, as judged by visual observation. Actual solubilities may be greater because of the volume of solvent portions utilized or a slow rate of dissolution. If dissolution did not occur as determined by visual assessment, the value was reported as “<”. If dissolution occurred at the first aliquot, the value was reported as “>”

Compound 1 Lot I demonstrated the highest solubility (>59 mg/mL) in acetic acid; moderate solubility (17 mg/mL) in TFE, and limited solubility (1-8 mg/mL) in acetone, chloroform, MEK, and aprotic solvents DMSO, DMAc, and NMP. Practically no solubility was observed in most common organic solvents, as well as in water. The addition of 10% water to acetone, ethanol, and methanol resulted in a slight increase in solubility (1-2 mg/mL). The approximate solubility estimates were used to design the experimental conditions for the polymorph screening activities.

The results of the approximate solubility estimate experiments are summarized in Table 2.1:

TABLE 2.1

| Solvent/Solvent System (v/v) | Temperature | Solubility (mg/mL) |
|---|---|---|
| AcOH | ambient | >59^{b} |
| Acetone | ambient | 1 |
| MeCN | ambient | <1 |
| 2-butanone (MEK) | ambient | 1 |
| CHCl₃ | ambient | 2 |
| DMA | ambient | 8 |
| DMSO | ambient | 6 |
| EtOAc | ambient | <1 |
| i-PrOH | ambient | <1 |
| MeOH | ambient | <1 |
| MIBK | ambient | <1 |
| NMP | ambient | 8 |
| THF | ambient | <1 |
| TFE | ambient | 17 |
| toluene | ambient | <1 |
| H₂O | ambient | <1 |
| MeOH:CHCl₃ (50:50) | ambient | <1 |
| MeOH:DCM (1:3) | ambient | 37 |
| AcOH:H₂O (20:80) | ambient | 1 |
| a_{w} = 0.94^{a} | | |
| AcOH:H₂O (50:50) | ambient | 14 |
| a_{w} = 0.84^{a} | | |
| AcOH:H₂O (85:15) | ambient | >51^{b} |
| a_{w} = 0.51^{a} | | |
| acetone:H₂O (90:10) | ambient | 2 |
| a_{w} = 0.69^{a} | | |
| 1-BuOH:H₂O (90:10) | 80° C. | 4^{c} |
| a_{w} = 0.82^{a} | | |
| 1-BuOH:H₂O (96:4) | 85° C. | 1^{c} |
| a_{w} = 0.49^{a} | | |
| EtOH:H₂O (90:10) | ambient | 1 |
| a_{w} = 0.49^{a} | | |
| MeOH:H₂O (90:10) | ambient | 1 |
| a_{w} = 0.29^{a} | | |

^{a}Water activity values calculated using UNIFAC calculator at RT.
^{b}After 6 days at RT, solution became dark purple or blue green, suggesting a potential degradation.
^{c}Compound 1 Lot II used as the starting material.

### Example 3. Polymorph Screening of Compound 1

### Slurry Experiments

Compound 1 Lot I was used as the starting material in a series of slurry experiments. Generally, slurries of Compound 1 Lot I (approx. 50 mg) were prepared by adding Compound 1 Lot I to a given solvent or solvent mixture at ambient or elevated temperature, such that undissolved solids were present. The resulting suspensions were then stirred for an extended period of time, and the solids were collected and analyzed. The experimental conditions and results are detailed in Table 3.1.

Approximately 15 slurries were performed at ambient temperature and the majority of them resulted in mixtures of crystalline phases. Five of the slurries produced solids with XRPD patterns that were successfully indexed indicating that these materials were composed primarily of single crystalline phases.

Compound 1 Form B was the most observed form in the slurry experiments, indicating that Form B is a stable anhydrous form at ambient temperature. Form B was also observed as a single phase in organic solvent/water mixtures with water activities up to 0.49 or 49% RH. Other forms isolated during the slurry experiments include Form D, Form E, Form F and Form N, all of which appeared to be solvated.

TABLE 3.1

| Solvent/Solvent | | | Sample |
|---|---|---|---|
| System (v/v) | Conditions | XRPD | ID |
| anhydrous acetone | Slurry, RT, 11 days | Form D | |
| acetone | Slurry, RT, 5 days | Form D | 3.1.1 |
| anhydrous MEK | Slurry, RT, 11 days | Form B + minor D | |
| anhydrous CHCl₃ | Slurry, RT, 11 days | Similar to Form G + peaks | |
| DCM | Slurry, RT, 4 days | Form L | |
| DMA | Slurry, RT, 11 days | Form B | |
| DMF | Slurry, RT, 11 days | Form B | |
| anhydrous DMSO | Slurry, RT, 5 days | Form E | |
| MeOH | Slurry, RT, 10 days | Form B + minor C | |
| anhydrous NMP | Slurry, RT, 5 days | Form N | |
| DMSO:H₂O (85:15) | Slurry, RT, 5 days | Form B + halo from solvent | 3.1.2 |
| a_{w} = 0.19^{a} | | | |
| — | Sample 3.1.2, VO, 42° C., 18 h | Form B | |
| EtOH:H₂O (90:10) | Slurry, RT, 10 days | Form B | |
| a_{w} = 0.49^{a} | | | |
| EtOH:H₂O (75:25) | Slurry, RT, 7 days | Form B + very minor C | |
| a_{w} = 0.76^{a} | Slurry, RT, +7 days; total 14 days | Form B + very minor C | |
| EtOH:H₂O (40:60) | Slurry, RT, 7 days | Form B + minor C | |
| a_{w} = 0.91^{a} | Slurry, RT, +7 days; total 14 days | Form B + minor C | |
| AcOH:H₂O (30:70) | Slurry, RT, 5 days | Form F + tiny peaks | |
| a_{w} = 0.91^{a} | Slurry, RT, 7 days; washed with EtOH | Form F | 3.1.3 |

^{a}Water activity values calculated using UNIFAC calculator at RT.

### Additional Polymorph Screening

Additional polymorph screening was performed and consisted of ten solution-based experiments employing various crystallization techniques, such as evaporation (rotary, fast, and slow at ambient conditions), crash precipitation (direct or reverse antisolvent addition), and vapor diffusion. Exemplary procedures for each technique are as follows:

Rotary Evaporation (RE): A solution of Compound 1 (approx. 50 mg) was prepared in a solvent system at ambient temperature. The solution was filtered and evaporated to dryness using a rotary evaporator. The solids were submitted for XRPD analysis.

Fast Evaporation (FE): Solutions of Compound 1 (approx. 50 mg) were prepared in various solvents or solvent systems at ambient temperature. The solutions were allowed to evaporate from open vials at ambient conditions. The solids were submitted for XRPD analysis.

Slow Evaporation (SE): Solutions of Compound 1 (approx. 50 mg) were prepared in various solvents or solvent systems at ambient temperature. The solutions were allowed to slowly evaporate from vials covered with perforated aluminum foil at ambient conditions. The solids were submitted for XRPD analysis.

Crash Precipitation (CP) or Solvent/Antisolvent Additions-Direct Addition: Solutions of Compound 1 (approx. 50 mg) were prepared in a given solvent or solvent combination and antisolvent was added portionwise. The resulting suspensions were stirred at ambient temperature prior to solid isolation. Solids were submitted for XRPD analysis.

Crash Precipitation (CP) or Solvent/Antisolvent Additions-Reverse Addition: Solutions of Compound 1 (approx. 50 mg) were prepared in various solvents at ambient temperature and filtered into a large amount of antisolvent at ambient temperature. The resulting solids were isolated for XRPD analysis.

Vapor Diffusion: Solutions of Compound 1 (approx. 50 mg) were prepared in solvent systems at ambient temperature. The solutions were filtered in new vials. Each vial was placed open in larger chamber containing antisolvent. The chamber was closed to allow vapor diffusion to occur.

Most experiments produced mixtures of crystalline phases. The experimental conditions and results are detailed in Table 3.2.

TABLE 3.2

| Solvent/ | | | | |
|---|---|---|---|---|
| Solvent System | | | | Sample |
| (v/v) | Conditions | Observation | XRPD Result | ID |
| MeOH:DCM | SE | Tan and yellow ppt, needles, | Form B + Form C | |
| (1:3) | | blades, irregular formations, BR | | |
| MeOH:DCM | CP w/EtOAc | Pale yellow; thin fibers in | Form B + peaks | |
| (1:3) | direct addition | balls aggregates, not BR | | |
| | CP w/EtOAc | Pale yellow, thin long single | Form G + peaks | |
| | reverse addition | needles, BR | | |
| | RE | Tan solids | Form A + peaks | |
| | VD in EtOAc | Tan, rhombic plates and | Form G | 3.2.1 |
| | | needles, BR | | |
| | VD in EtOAc | Tan, equants and rods, BR | Form A w/slight | 3.2.2 |
| | Scale up | | disorder (sample | |
| | | | ground)^{b} | |
| | VD in EtOAc | Tan, equants and rods, BR | Forms P + Q | 3.2.3 |
| | saturated w/H₂O | | | |
| | VD in EtOAc | Tan, blades and large | Form G + peaks of | |
| | Scale up | hexagonal plates, BR | Form P | |
| anhydrous | Slow evaporation | Dark tan, blades and plates, | Form O + some | |
| TFE:EtOAc | | BR | disorder | |
| (90:10) | | | | |
| TFE:H₂O | Fast evaporation | Tan, equants, aggregated, BR | Form O | |
| (95:5) | | | | |

### Exposure Experiments

In a series of exposure experiments, Compound 1 Lot I was exposed to high relative humidity. Generally, approx. 50 mg Compound 1 was transferred to vials, and the vials were placed open in chambers containing saturated aqueous solutions of various salts, which are known to generate various relative humidity levels in the chambers. See, e.g., “Standard Practice for Maintaining Constant Relative Humidity by Means of Aqueous Solutions” ASTM Standard E 104-85 (1996); “Humidity Fixed Points of Binary Saturated Aqueous Solutions” Greenspan, L. J. Res. Natl. Bur. Stand., Sect. A 1997, 81A, 89-96; “Saturated Salt Solutions for Maintaining Specified Relative Humidities” Nyqvist, H. E. Int. J. Pharma. Technol. Prod. Manuf. 1983, 4, 47-48; and “Evaluation of Relative Humidity Values for Saturated Aqueous Salt Solutions Using Osmotic Coefficients Between 50 and 100° C.” Moisture Humidity, Proc. Int. Symp. 1985, 577-96.

The exposure experiments resulted in mixtures of forms, and no single crystalline phase of a hydrated material was produced through the exposure experiments. The experimental conditions and results are detailed in Table 3.3.

TABLE 3.3

| Starting Material | Conditions | XRPD Result |
|---|---|---|
| Compound 1 Lot I | 75% RH, RT, 17 days | Forms B + C + larger peaks |
| | 0.56% weight gain | |
| | 97% RH, RT, 17 days | Forms B + C + peaks |
| | 0.71% weight gain | |

### Scale-Up Experiments

Scale-up slurry experiments were performed to produce larger quantities of certain solid forms described herein. Generally, slurries of Compound 1 (approx. 400-600 mg) were prepared by adding Compound 1 to a given solvent or solvent mixture at ambient or elevated temperature, such that undissolved solids were present. The resulting suspensions were then stirred for an extended period of time, and the solids were collected and analyzed.

Scale-up slurry experiments targeting Compound 1 Form B, Compound 1 Form C, Compound 1 Form D, and Compound 1 Form F were successfully conducted, as summarized in Table 3.4.

TABLE 3.4

| | | | Sample |
|---|---|---|---|
| Starting Form | Conditions | XRPD Result | ID |
| Compound 1 | acetone, slurry, RT, 6 days | Form D | 3.4.1 |
| Lot II | acetone, slurry, RT, 5 days | Form D | 3.4.2 |
| | acetone, slurry, RT, 5 days | Form D + minor C | 3.4.3 |
| | AcOH:H₂O (30:70), slurry, RT, 3 days | Form F + minor Form B | 3.4.4 |
| | AcOH:H₂O (30:70), slurry, RT, +2 days; | Form F | |
| | total 5 days | | |
| | EtOH:H₂O (90:10); slurry, RT, 3 days; VF, | Form B | 3.4.5 |
| | washed w/EtOH; VO, 45° C., 19 hours | | |
| | Subsample of suspension Sample 3.4.5 | Form B | |
| Compound 1 | Acetone, slurry, RT, 7 days | Form D | 3.4.6 |
| Lot I | EtOH:H₂O (90:10); slurry, RT, 10 days; | Form B | |
| | washed with EtOH VO, 50° C., 5 hours | | |
| | AcOH:H₂O (30:70); slurry, RT, 7 days; | Form F | |
| | washed with EtOH | | |
| Sample 3.4.4 | H₂O, slurry, RT, 7 days | Form C | |
| (Form F + | | | |
| minor Form B) | | | |

Several scale-up vapor diffusion experiments targeting Compound 1 Form G were conducted successfully. Generally, solutions of Compound 1 (approx. 400-600 mg) were prepared in solvent systems at ambient temperature. The solutions were filtered in new vials. Each vial was placed open in larger chamber containing antisolvent. The chamber was closed to allow vapor diffusion to occur.

One of the experiments resulted in Form G with very minor amount of Form P. The results of the scale-up vapor diffusion experiments are summarized in Table 3.5.

TABLE 3.5

| Solvent/Solvent | | | | Sample |
|---|---|---|---|---|
| System (v/v) | Conditions | Observation | XRPD Result | ID |
| MeOH:DCM | VD in EtOAc | Tan, large plates, BR | Form G | 3.5.1 |
| (1:3) | VD in EtOAc | Tan, hexagonal large plates, B and | Form G | 3.5.2 |
| | | rods, BR | | |
| | VD in EtOAc | Tan, large plates & blades, BR | Form G | 3.5.3 |
| | VD in EtOAc | Tan, irregular large plates, rods & | Form G + peaks | 3.5.4 |
| | | needles, BR | of Form P | |

### Heating Experiments

In an effort to obtain anhydrous and non-solvated materials, heating experiments were performed on solvated and hydrated forms based on the thermal data of each form. The heating experiments resulted in mixtures of crystalline materials such as Form B and Form I or Form H and Form I. No single crystalline phase material was produced by the heating experiments. The results of the heating experiments are summarized in Table 3.6.

TABLE 3.6

| Starting Material | Conditions | XRPD Result | Sample ID |
|---|---|---|---|
| Sample 3.2.2 (Form A) | Heated to 195° C. | Form B + Form I | |
| Sample 3.1.1 (Form D) | Heated to 195° C. | Forms H + I | 3.6.1 |
| Sample 3.4.1 (Form D) | Heated to 200° C. | Forms I + H | 3.6.2 |
| Sample 3.4.2 (Form D) | Heated to 200° C. | Forms I + H | 3.6.3 |
| | | Forms I + H | 3.6.4 |
| Sample 3.2.1 (Form G) | Heated to 180° C. | Form H + minor Form I | |
| Sample 3.4.3 (Forms D + minor C) | Heated to 210° C. | Form B + minor Form I + peaks | |

### Solvent-Based Conversion Experiments

Solvent-based conversion experiments were performed on solvated forms in bulky solvents in an effort to obtain anhydrous and non-solvated materials. Generally, slurries of Compound 1 (approx. 50 mg) were prepared by adding Compound 1 to a given solvent or solvent mixture at ambient or elevated temperature, such that undissolved solids were present. The resulting suspensions were then stirred for an extended period of time, and the solids were collected and analyzed. The experimental conditions are detailed in Table 3.7.

Most of these experiments resulted in mixtures of Form B and Form H and/or Form I or in new solvated materials, such as Form J, Form K, and Form M. Form H was produced as solids exhibiting an XRPD pattern characteristic of crystalline material with disorder that prevented its indexing.

TABLE 3.7

| Starting Form | Conditions | XRPD Result | Sample ID |
|---|---|---|---|
| Sample 3.4.6 | CPME, slurry, 50° C., 3 days | Form B + Form I | 3.7.1 |
| (Form D) | TAME, slurry, RT, 3 days | Form B + Forms H + I | |
| Sample 3.4.1 | TAME, slurry, 65° C., 3 days | Form B + minor Form I | |
| (Form D) | Bu₂O, slurry, 65° C., 3 days | Form B + very minor Form I | |
| | Bu₂O, slurry, 65° C., 19 h | Form B + minor Form I | |
| Sample 3.4.2 | Bu₂O, slurry, RT, 4 days | Forms H + minor I | |
| (Form D) | Bu₂O, slurry, RT, +5 days | Forms H + minor I + minor Form B | |
| | Bu₂O, slurry, 35° C., 5 days | Form K (Bu₂O solvate) | |
| | H₂O, slurry, RT, 4 days | Form C + peaks | |
| Sample 3.1.3 | H₂O, slurry, RT, 3 days | Form C | |
| (Form F) | CPME, 50° C., 3 days | Form M (CPME solvate) | |
| Sample 3.5.1 | TAME, slurry, 50° C., 4 days | Form H + very minor Form I | |
| (Form G) | | disordered | |
| | TAME, slurry, RT, +6 days | Form H disordered | 3.7.2 |
| Sample 3.7.2 | EtOH:H₂O 90:10, RT, 3 days | Form H disordered | |
| (Form H) | | | |
| Sample 3.5.3 | TAME, slurry, 35° C., 6 days | Forms I + H | |
| (Form G) | | | |
| Sample 3.5.2 | MTBE, slurry, RT, 6 days | Form J (MTBE solvate) | |
| (Form G) | | | |

### Conclusions

Overall, approximately 75 experiments were carried out as part of the polymorph screening activities producing eighteen new crystalline phases (see FIG. 3A, FIG. 3B, and FIG. 3C). The XRPD patterns of 12 of them were successfully indexed, indicating that each is composed primarily or exclusively of a single crystalline phase. The discovery of so many new crystalline phases evidenced the strong tendency of Compound 1 to polymorphism-forming anhydrous, hydrated, and solvated materials characteristic of a complex phase space.

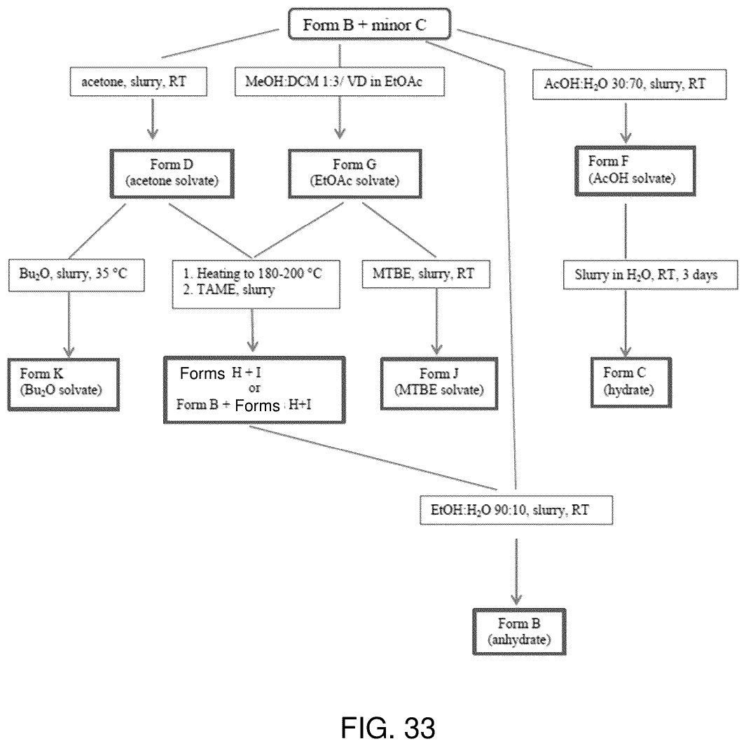

FIG. 33 provides a summary of various forms of Compound 1 identified in these experiments.

### Example 4. Interconversion Slurry Experiments

Interconversion slurry experiments were performed with Compound 1 Form B, Compound 1 Form H, and Compound 1 Form I in an effort to determine the most stable anhydrous form at various temperatures, including ambient temperature.

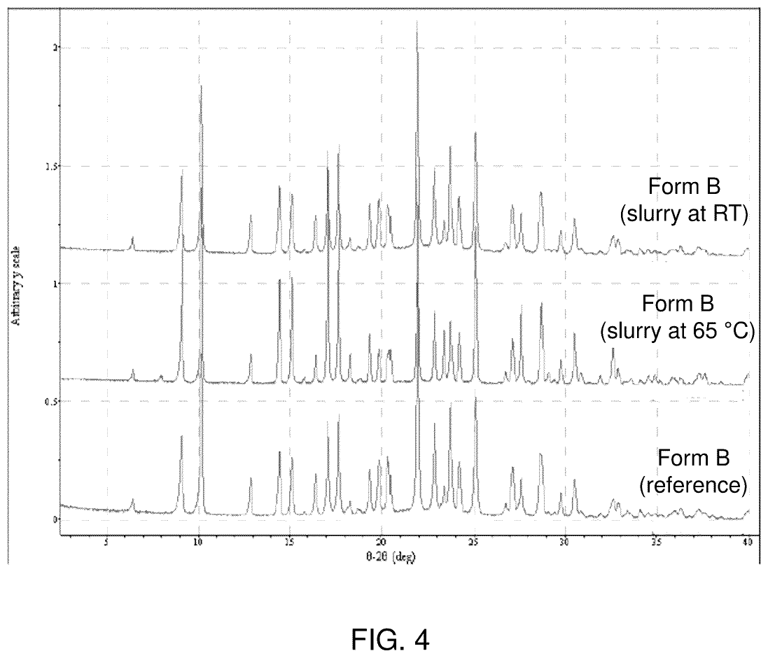

Mixtures of Form H and Form I with Form B were utilized in these interconversion slurry experiments. The experimental conditions are detailed in Table 4.1. The interconversion slurries were performed at ambient and elevated (65° C.) temperatures in various solvents and solvent mixtures. Slurries at both temperatures resulted in Form B; however, the solids from elevated temperature slurry experiments exhibited an additional small peak at ˜8.0 °2θ (FIG. 4). HPLC analysis of material containing Form B and the small peak at ˜8.0 °2θ indicated a small amount of degradation of Compound 1, which suggested that the small peak represented an unidentified degradation product.

The results from the interconversion slurry experiments demonstrated that no form other than Form B resulted from the experiments at ambient and elevated temperature. Form B is likely the most stable anhydrous form under the tested conditions.

TABLE 4.1

| Starting Form | Conditions | XRPD Result | Sample ID |
|---|---|---|---|
| Sample 3.7.1 (Form B + Form I) + | TAME, slurry, 80° C., 3 days - | Form B + Forms H + I | 4.1.1 |
| Sample 3.6.1 (Forms H + I) | dried out | | |
| Sample 4.1.1 (Form B + Forms | EtOH:H₂O (90:10); slurry, | Form B | |
| H + I) | RT, 9 days | | |
| Compound 1 Lot II (Form B) + | EtOH:H₂O (90:10); slurry, | Form B + small peak | |
| Sample 3.6.2 (Forms H + I) | 65° C., 6 days | at ~8.0°2θ | |
| | EtOH:H₂O (90:10); slurry, | Form B + small peak | |
| | 65° C., +3 days; total 9 days | at ~8.0°2θ | |
| Compound 1 Lot II (Form B) + | EtOH:H₂O (90:10); slurry, | Form B + small peak | |
| Sample 3.6.3 (Forms H + I) | 65° C., 3 days | at ~8.0°2θ | |
| | EtOH:H₂O (90:10); slurry, | Form B + small peak | 4.1.2 |
| | 65° C., +3 days; total 6 days | at ~8.0°2θ^{b} | |
| Form B + Sample 3.6.4 (Forms | EtOH:H₂O (90:10); slurry, | Form B + small peak | |
| H + I) | 65° C., 3 days | at ~8.0°2θ | |
| | EtOH:H₂O (90:10); slurry, | Form B + small peak | 4.1.3 |
| | 65° C., +3 days; total 6 days | at ~8.0°2θ^{c} | |
| Sample 4.1.3 (Form B + small | Anhydrous TAME, slurry, | Form B + small peaks | |
| peak at ~8.0°2θ) | 65° C., 3 days | | |
| Sample 4.1.2 (Form B) | 1-BuOH:H₂O (96:4); slurry, | Form B + very minor | |
| | 80° C., 3 days | Form S* | |
| | 1-BuOH:H₂O (96:4); slurry, | Form B + minor Form | |
| | 85° C., +5 days | S* | |
| | 1-BuOH:H₂O (96:4); slurry, | Form B + Form S* | |
| | 90° C., +6 days | | |

*Material containing Form S was determined to comprise a meaningful amount of a degradation product (n-butyl carbamate); thus, Form S is likely a degradation product and not Compound 1.

### Example 5. Compound 1 Form A

Compound 1 Form A was prepared by vapor diffusion of a solution of Compound 1 (approx. 50 mg) in MeOH:DCM (1:3, v/v) in vapor of ethyl acetate. The solids were isolated and analyzed.

FIG. 3A (top spectrum) provides an XRPD spectrum of Compound 1 Form A. XRPD Data corresponding to Compound 1 Form A are summarized in Table 5.1.

TABLE 5.1

| °2θ | d-spacing (Å) |
|---|---|
| 7.38 ± 0.20 | 11.968 ± 0.324 |
| 8.53 ± 0.20 | 10.353 ± 0.242 |
| 9.40 ± 0.20 | 9.401 ± 0.200 |
| 11.05 ± 0.20 | 8.001 ± 0.144 |
| 11.99 ± 0.20 | 7.375 ± 0.123 |
| 12.42 ± 0.20 | 7.122 ± 0.114 |
| 13.48 ± 0.20 | 6.563 ± 0.097 |
| 14.31 ± 0.20 | 6.184 ± 0.086 |
| 14.98 ± 0.20 | 5.910 ± 0.078 |
| 15.58 ± 0.20 | 5.682 ± 0.072 |
| 16.03 ± 0.20 | 5.525 ± 0.068 |
| 17.37 ± 0.20 | 5.103 ± 0.058 |
| 17.72 ± 0.20 | 5.000 ± 0.056 |
| 18.39 ± 0.20 | 4.821 ± 0.052 |
| 18.80 ± 0.20 | 4.717 ± 0.050 |
| 19.42 ± 0.20 | 4.567 ± 0.047 |
| 20.68 ± 0.20 | 4.291 ± 0.041 |
| 21.90 ± 0.20 | 4.054 ± 0.037 |
| 23.34 ± 0.20 | 3.809 ± 0.032 |
| 23.66 ± 0.20 | 3.758 ± 0.031 |
| 24.06 ± 0.20 | 3.696 ± 0.030 |
| 24.99 ± 0.20 | 3.560 ± 0.028 |
| 25.53 ± 0.20 | 3.487 ± 0.027 |
| 26.02 ± 0.20 | 3.421 ± 0.026 |
| 27.12 ± 0.20 | 3.286 ± 0.024 |
| 28.81 ± 0.20 | 3.096 ± 0.021 |

### Example 6. Compound 1 Form B

Form B was observed in Compound 1 Lot I and Lot II. Additionally, Form B was prepared by slurrying Compound 1 in DMA, DMF, DMSO:H₂O 85:15 (v/v) or EtOH:H₂O 90:10 (v/v) for 5-11 days, as detailed in Table 3.1.

FIG. 5 provides an XRPD spectrum of Compound 1 Form B. The XRPD pattern of Form B exhibited well-resolved peaks with high intensity, characteristic of crystalline material. The XRPD pattern of Form B was successfully indexed indicating that the material is composed primarily or exclusively of a single crystalline phase. Based on the calculated volume from the indexing solution, the unit cell is characteristic of an anhydrous/non-solvated material. XRPD data corresponding to Compound 1 Form B are summarized in Table 6.1.

TABLE 6.1

| °2θ | d-spacing (Å) |
|---|---|
| 6.40 ± 0.20 | 13.799 ± 0.431 |
| 9.05 ± 0.20 | 9.764 ± 0.215 |
| 9.09 ± 0.20 | 9.721 ± 0.213 |
| 10.16 ± 0.20 | 8.699 ± 0.171 |
| 12.85 ± 0.20 | 6.884 ± 0.107 |
| 14.42 ± 0.20 | 6.138 ± 0.085 |
| 15.10 ± 0.20 | 5.863 ± 0.077 |
| 15.79 ± 0.20 | 5.608 ± 0.071 |
| 16.39 ± 0.20 | 5.404 ± 0.065 |
| 17.08 ± 0.20 | 5.187 ± 0.060 |
| 17.66 ± 0.20 | 5.018 ± 0.056 |
| 18.28 ± 0.20 | 4.849 ± 0.053 |
| 18.72 ± 0.20 | 4.736 ± 0.050 |
| 19.35 ± 0.20 | 4.583 ± 0.047 |
| 19.85 ± 0.20 | 4.469 ± 0.045 |
| 20.35 ± 0.20 | 4.360 ± 0.042 |
| 21.89 ± 0.20 | 4.057 ± 0.037 |
| 22.83 ± 0.20 | 3.892 ± 0.034 |
| 23.37 ± 0.20 | 3.803 ± 0.032 |
| 23.71 ± 0.20 | 3.750 ± 0.031 |
| 24.20 ± 0.20 | 3.675 ± 0.030 |
| 25.09 ± 0.20 | 3.546 ± 0.028 |
| 26.73 ± 0.20 | 3.332 ± 0.024 |
| 27.10 ± 0.20 | 3.288 ± 0.024 |
| 27.57 ± 0.20 | 3.233 ± 0.023 |
| 28.59 ± 0.20 | 3.120 ± 0.021 |
| 28.67 ± 0.20 | 3.111 ± 0.021 |
| 29.06 ± 0.20 | 3.070 ± 0.021 |
| 29.37 ± 0.20 | 3.038 ± 0.020 |
| 29.75 ± 0.20 | 3.001 ± 0.020 |
| 30.49 ± 0.20 | 2.929 ± 0.019 |
| 30.85 ± 0.20 | 2.896 ± 0.018 |
| 31.92 ± 0.20 | 2.802 ± 0.017 |
| 32.59 ± 0.20 | 2.745 ± 0.016 |
| 32.87 ± 0.20 | 2.723 ± 0.016 |

The peak positions and integration values in the proton NMR spectrum of Form B prepared in ethanol:water 90:10 (v/v) were consistent with the chemical structure of Compound 1. Traces of ethanol and ethyl acetate were also detected in the spectrum.

The DSC thermogram of Form B obtained by slurry in ethanol:water 90:10 (v/v) demonstrated a large endotherm with peak maximum at 285.2° C. and onset at 278.7° C. (FIG. 6). The TGA curve showed an insignificant weight loss of ˜0.04% between 54° C. and 150° C., indicative of the anhydrous/non-solvated nature of Form B (FIG. 6).

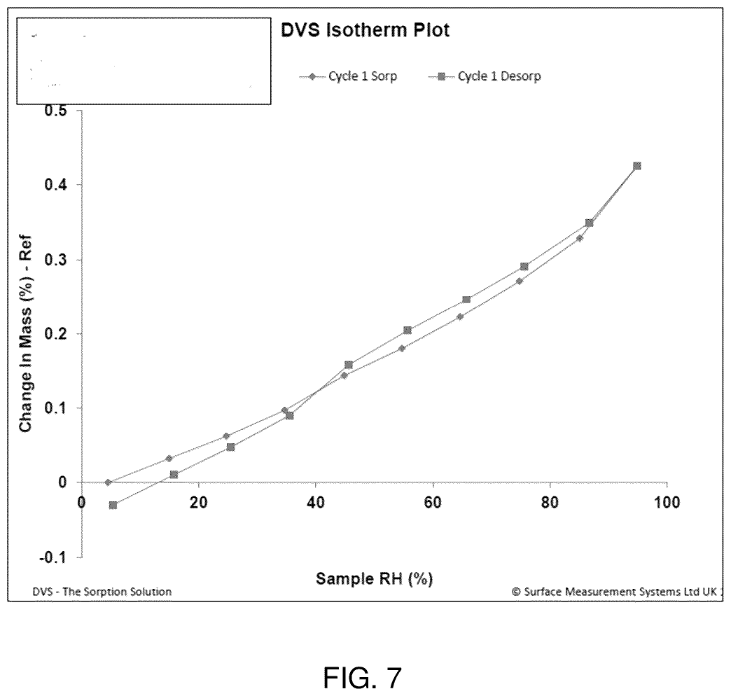

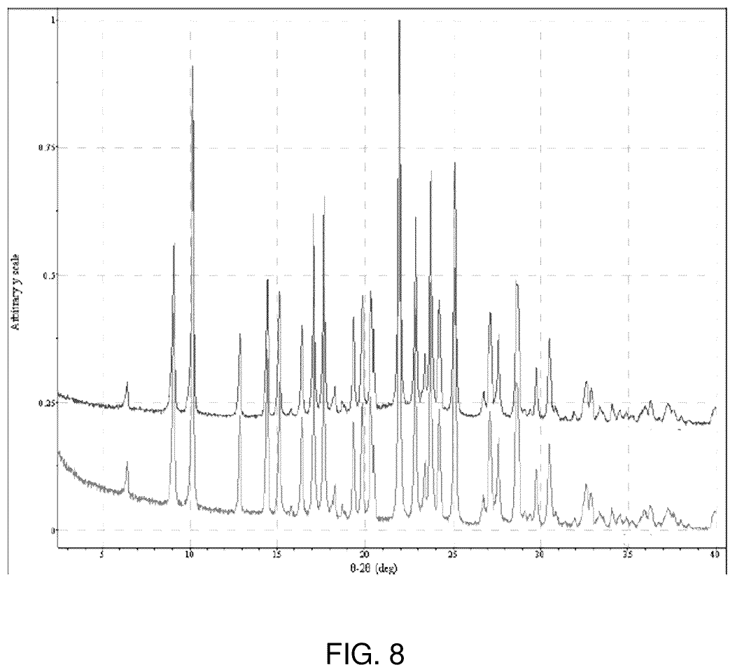

A DVS experiment was performed on Form B (FIG. 7 and Table 6.2). No weight change was observed upon equilibration at 5% RH. Thereafter, the material experienced a small gradual weight gain of 0.43% at 5-95% RH, which was lost during the desorption step at 95-5% RH. The insignificant weight changes during the sorption/desorption steps indicated that Form B was non-hygroscopic. The XRPD pattern of the post-DVS sample is consistent with Form B (FIG. 8) suggesting that Form B remained unchanged during the DVS experiment.

TABLE 6.2

| | | | | | Desorp | |
|---|---|---|---|---|---|---|
| | | | Sorp Mass | | Mass | |
| | Target | Sample | Change | Sample | Change | |
| | RH (%) | RH (%) | (%) | RH (%) | (%) | Hysteresis |
| Cycle 1 | 5 | 4.5 | 0.0003 | 5.3 | −0.0297 | |
| | 15 | 14.9 | 0.0327 | 15.7 | 0.0109 | −0.0218 |
| | 25 | 24.7 | 0.0627 | 25.5 | 0.048 | −0.0147 |
| | 35 | 34.7 | 0.0974 | 35.5 | 0.0903 | −0.0071 |
| | 45 | 44.9 | 0.1439 | 45.6 | 0.1586 | 0.0147 |
| | 55 | 54.7 | 0.1803 | 55.7 | 0.2045 | 0.0241 |
| | 65 | 64.7 | 0.2227 | 65.7 | 0.2463 | 0.0235 |

Stability stress studies were conducted on Form B by exposing samples to three conditions: 57.5% RH/RT; 97.3% RH/RT, and 75% RH/40° C. and collecting XRPD data at two time points (14 and 28 days). The experimental conditions are summarized in Table 6.3. Samples analyzed under all conditions and at all time points were consistent with Form B, suggesting that Form B is stable at the tested stress conditions for the duration of the study.

TABLE 6.3

| Starting Form | Conditions | XRPD Result |
|---|---|---|
| Form B | 57.5% RH/RT, 14 days | Form B |
| | 57.5% RH/RT, 28 days | Form B |
| | 97.3% RH/RT, 14 days | Form B |
| | 97.3% RH/RT, 28 days | Form B |
| | 75.3% RH/40° C., 14 days | Form B |
| | 75.3% RH/40° C., 28 days | Form B |

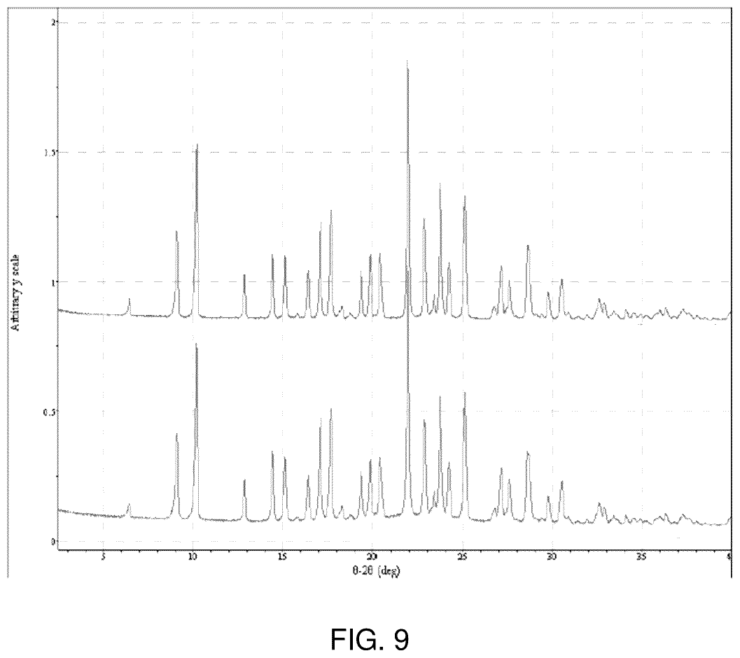

A slurry of Form B was carried out in water at ambient temperature for 5 days, and Form B remained unchanged, as judged by XRPD analysis (FIG. 9).

Based on the characterization data described above, Form B was assigned as an anhydrous, non-solvated form of Compound 1.

### Example 7. Compound 1 Form C

Compound 1 Form C was obtained by slurrying Compound 1 Form F in water at ambient temperature for 3 days, as described in Table 3.7.

FIG. 10 provides an XRPD spectrum of Compound 1 Form C. The XRPD pattern of Form C exhibited well-resolved peaks with high intensity, characteristic of crystalline material. The XRPD pattern of Form C was successfully indexed indicating that the material is composed primarily or exclusively of a single crystalline phase. Based on the calculated volume from the indexing solution, the unit cell could accommodate up to three or four molecules of water per one molecule of Compound 1. XRPD data corresponding to Compound 1 Form C are summarized in Table 7.1.

TABLE 7.1

| °2θ | d-spacing (Å) |
|---|---|
| 7.92 ± 0.20 | 11.154 ± 0.281 |
| 10.10 ± 0.20 | 8.751 ± 0.173 |
| 10.36 ± 0.20 | 8.532 ± 0.164 |
| 10.89 ± 0.20 | 8.118 ± 0.149 |
| 11.44 ± 0.20 | 7.729 ± 0.135 |
| 12.02 ± 0.20 | 7.357 ± 0.122 |
| 12.57 ± 0.20 | 7.036 ± 0.112 |
| 13.10 ± 0.20 | 6.753 ± 0.103 |
| 13.53 ± 0.20 | 6.539 ± 0.096 |
| 14.13 ± 0.20 | 6.263 ± 0.088 |
| 14.42 ± 0.20 | 6.138 ± 0.085 |
| 15.47 ± 0.20 | 5.723 ± 0.074 |
| 15.90 ± 0.20 | 5.569 ± 0.070 |
| 16.71 ± 0.20 | 5.301 ± 0.063 |
| 16.98 ± 0.20 | 5.218 ± 0.061 |
| 17.40 ± 0.20 | 5.093 ± 0.058 |
| 17.86 ± 0.20 | 4.962 ± 0.055 |
| 18.07 ± 0.20 | 4.905 ± 0.054 |
| 18.54 ± 0.20 | 4.782 ± 0.051 |
| 18.82 ± 0.20 | 4.711 ± 0.050 |
| 19.23 ± 0.20 | 4.612 ± 0.048 |
| 19.73 ± 0.20 | 4.496 ± 0.045 |
| 19.94 ± 0.20 | 4.449 ± 0.044 |
| 20.17 ± 0.20 | 4.399 ± 0.043 |
| 20.82 ± 0.20 | 4.263 ± 0.041 |
| 22.17 ± 0.20 | 4.006 ± 0.036 |
| 22.58 ± 0.20 | 3.935 ± 0.034 |
| 23.03 ± 0.20 | 3.859 ± 0.033 |
| 23.40 ± 0.20 | 3.799 ± 0.032 |
| 23.97 ± 0.20 | 3.709 ± 0.030 |
| 24.19 ± 0.20 | 3.676 ± 0.030 |
| 24.85 ± 0.20 | 3.580 ± 0.028 |
| 25.79 ± 0.20 | 3.452 ± 0.026 |
| 26.35 ± 0.20 | 3.380 ± 0.025 |
| 27.23 ± 0.20 | 3.272 ± 0.024 |

The peak positions and integration values in the proton NMR spectrum of Form C were consistent with the chemical structure of Compound 1. Trace acetic acid and other unidentified impurities were observed in the spectrum.

The DSC thermogram displayed a broad endotherm with peak maximum at 143.7° C., followed by an exotherm with peak maximum at 190.5° C. and an onset at 187.1° C. (FIG. 11). An endotherm with peak maximum at 277.2° C. and an onset at 265.2° C. was also observed in the DSC thermogram. The TGA curve indicated 5.8% weight loss between 54° C. and 180° C. that accompanied the broad endotherm and corresponded to 1.8 moles of H₂O (FIG. 11).

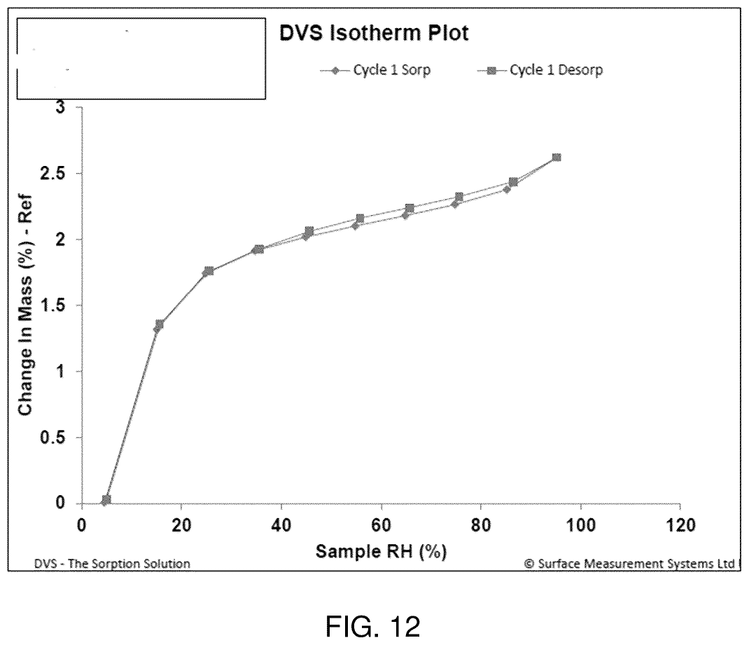

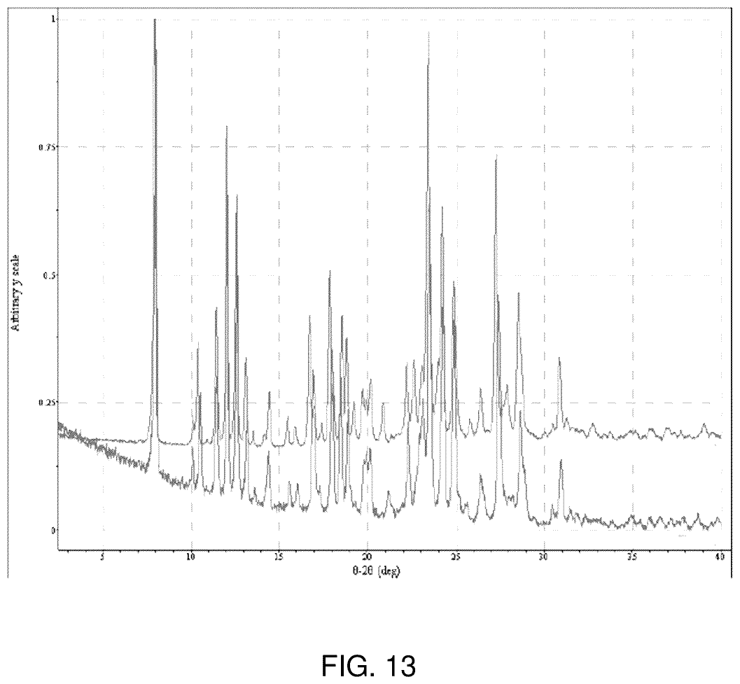

A DVS experiment was performed on Form C (FIG. 12 and Table 7.2). No weight change was observed upon equilibration at 5% RH. Thereafter, the material experienced rapid weight gain of 1.74% (0.5 mole of H₂O; total 2.3 moles of H₂O) from 5-25% RH followed by a slight gradual increase up to 2.6% (0.8 mole of H₂O; total 2.6 moles of H₂O) at 95% RH, which was lost on the desorption step 95-5% RH, leaving 1.8 moles of water in the resulting post-DVS material. The XRPD pattern of the post-DVS sample is consistent with Form C with slight peak shifting (FIG. 13) suggesting that Form C experienced a slight change of the water content during the duration of the DVS experiment.

TABLE 7.2

| | | | | | Desorp | |
|---|---|---|---|---|---|---|
| | | | Sorp Mass | | Mass | |
| | Target | Sample | Change | Sample | Change | |
| | RH (%) | RH (%) | (%) | RH (%) | (%) | Hysteresis |
| Cycle 1 | 5.0 | 4.5 | 0.005 | 4.9 | 0.028 | |
| | 15.0 | 15.0 | 1.314 | 15.6 | 1.354 | 0.040 |
| | 25.0 | 24.8 | 1.741 | 25.5 | 1.757 | 0.016 |
| | 35.0 | 34.7 | 1.912 | 35.5 | 1.927 | 0.015 |
| | 45.0 | 44.9 | 2.017 | 45.6 | 2.061 | 0.044 |
| | 55.0 | 54.8 | 2.100 | 55.8 | 2.161 | 0.061 |
| | 65.0 | 64.9 | 2.178 | 65.6 | 2.240 | 0.061 |
| | 75.0 | 74.8 | 2.262 | 75.6 | 2.323 | 0.061 |
| | 85.0 | 85.2 | 2.375 | 86.5 | 2.437 | 0.062 |
| | 95.0 | 95.3 | 2.620 | 95.3 | 2.620 | |

Based on the characterization data described above, Form C was assigned as a hydrate form of Compound 1.

### Example 8. Compound 1 Form D

Compound 1 Form D was prepared by slurrying Compound 1 in acetone at ambient temperature for 11 days, as described in Table 3.1.

FIG. 14 provides an XRPD spectrum of Compound 1 Form D. The XRPD pattern of Form D exhibited well-resolved peaks with high intensity, characteristic of crystalline material. The XRPD pattern of Form D was successfully indexed indicating that the material is composed primarily or exclusively of a single crystalline phase. Based on the calculated volume from the indexing solution, the unit cell could accommodate up to three molecules of acetone per one molecule of Compound 1. XRPD data corresponding to Compound 1 Form D are summarized in Table 8.1.

TABLE 8.1

| °2θ | d-spacing (Å) |
|---|---|
| 7.27 ± 0.20 | 12.150 ± 0.334 |
| 8.31 ± 0.20 | 10.631 ± 0.255 |
| 8.47 ± 0.20 | 10.431 ± 0.246 |
| 10.33 ± 0.20 | 8.557 ± 0.165 |
| 11.87 ± 0.20 | 7.450 ± 0.125 |
| 12.58 ± 0.20 | 7.031 ± 0.111 |
| 12.99 ± 0.20 | 6.810 ± 0.104 |
| 13.56 ± 0.20 | 6.526 ± 0.096 |
| 14.04 ± 0.20 | 6.303 ± 0.089 |
| 14.58 ± 0.20 | 6.071 ± 0.083 |
| 16.11 ± 0.20 | 5.497 ± 0.068 |
| 16.36 ± 0.20 | 5.414 ± 0.066 |
| 16.57 ± 0.20 | 5.346 ± 0.064 |
| 17.24 ± 0.20 | 5.139 ± 0.059 |
| 17.79 ± 0.20 | 4.982 ± 0.056 |
| 18.01 ± 0.20 | 4.921 ± 0.054 |
| 18.72 ± 0.20 | 4.736 ± 0.050 |
| 18.95 ± 0.20 | 4.679 ± 0.049 |
| 19.58 ± 0.20 | 4.530 ± 0.046 |
| 20.03 ± 0.20 | 4.429 ± 0.044 |
| 20.77 ± 0.20 | 4.273 ± 0.041 |
| 21.52 ± 0.20 | 4.126 ± 0.038 |
| 21.98 ± 0.20 | 4.041 ± 0.036 |
| 22.05 ± 0.20 | 4.028 ± 0.036 |
| 22.89 ± 0.20 | 3.882 ± 0.033 |
| 23.57 ± 0.20 | 3.772 ± 0.032 |
| 23.85 ± 0.20 | 3.728 ± 0.031 |
| 24.32 ± 0.20 | 3.657 ± 0.030 |
| 24.74 ± 0.20 | 3.596 ± 0.029 |
| 25.07 ± 0.20 | 3.549 ± 0.028 |
| 25.33 ± 0.20 | 3.513 ± 0.027 |
| 25.68 ± 0.20 | 3.467 ± 0.027 |
| 26.17 ± 0.20 | 3.402 ± 0.026 |
| 26.55 ± 0.20 | 3.354 ± 0.025 |
| 26.85 ± 0.20 | 3.318 ± 0.024 |

The peak positions and integration values in the proton NMR spectrum of Form D were consistent with the chemical structure of Compound 1. Trace acetone and other unidentified impurities were detected in the sample.

The DSC thermogram of Form D displayed a small exotherm at 142.2° C., followed by a small endotherm with peak maximum at 257.5° C. and onset at 252.2° C., next to a large endotherm with peak maximum at 274.7° C. and onset at 268.7° C. (FIG. 15). The TGA curve showed a 10.05% weight loss between 53° C. and 165° C., which is equivalent to 1.015 mole of acetone (FIG. 15).

Based on the characterization data described above, Form D was assigned as an acetone monosolvate of Compound 1.

### Example 9. Compound 1 Form E

Compound 1 Form E was prepared by slurrying Compound 1 in DMSO at ambient temperature for 5 days, as described in Table 3.1.

FIG. 16 provides an XRPD spectrum of Compound 1 Form E. The XRPD pattern of Form E exhibited well-resolved peaks with high intensity characteristic of crystalline material. The XRPD pattern of Form E was successfully indexed indicating that the material is composed primarily or exclusively of a single crystalline phase. Based on the calculated volume from the indexing solution, the unit cell could accommodate up to two molecules of DMSO per one molecule of Compound 1. XRPD data corresponding to Compound 1 Form E are summarized in Table 9.1.

TABLE 9.1

| °2θ | d-spacing (Å) |
|---|---|
| 7.26 ± 0.20 | 12.166 ± 0.335 |
| 8.47 ± 0.20 | 10.431 ± 0.246 |
| 10.32 ± 0.20 | 8.565 ± 0.166 |
| 11.32 ± 0.20 | 7.810 ± 0.138 |
| 11.89 ± 0.20 | 7.437 ± 0.125 |
| 12.56 ± 0.20 | 7.042 ± 0.112 |
| 13.06 ± 0.20 | 6.773 ± 0.103 |
| 14.03 ± 0.20 | 6.307 ± 0.089 |
| 14.56 ± 0.20 | 6.079 ± 0.083 |
| 15.23 ± 0.20 | 5.813 ± 0.076 |
| 16.16 ± 0.20 | 5.480 ± 0.067 |
| 16.51 ± 0.20 | 5.365 ± 0.065 |
| 16.99 ± 0.20 | 5.214 ± 0.061 |
| 17.15 ± 0.20 | 5.166 ± 0.060 |
| 17.40 ± 0.20 | 5.093 ± 0.058 |
| 17.94 ± 0.20 | 4.940 ± 0.055 |
| 17.98 ± 0.20 | 4.930 ± 0.054 |
| 18.28 ± 0.20 | 4.850 ± 0.053 |
| 18.57 ± 0.20 | 4.774 ± 0.051 |
| 18.77 ± 0.20 | 4.724 ± 0.050 |
| 18.95 ± 0.20 | 4.679 ± 0.049 |
| 19.68 ± 0.20 | 4.507 ± 0.045 |
| 19.99 ± 0.20 | 4.438 ± 0.044 |
| 20.39 ± 0.20 | 4.352 ± 0.042 |
| 20.75 ± 0.20 | 4.277 ± 0.041 |
| 21.05 ± 0.20 | 4.217 ± 0.040 |
| 21.65 ± 0.20 | 4.101 ± 0.037 |
| 21.92 ± 0.20 | 4.052 ± 0.037 |
| 22.14 ± 0.20 | 4.012 ± 0.036 |
| 22.66 ± 0.20 | 3.921 ± 0.034 |
| 22.89 ± 0.20 | 3.882 ± 0.033 |
| 23.10 ± 0.20 | 3.847 ± 0.033 |
| 23.76 ± 0.20 | 3.742 ± 0.031 |
| 24.16 ± 0.20 | 3.681 ± 0.030 |
| 24.42 ± 0.20 | 3.642 ± 0.029 |

The peak positions and integration values in the proton NMR spectrum of Form E were consistent with the chemical structure of Compound 1. A singlet at 2.54 ppm was indicative of ˜2.1 moles of DMSO present in the material. Trace unidentified impurities were also observed.

Based on the characterization data described above, Form E was assigned as a DMSO disolvate of Compound 1.

### Example 10. Compound 1 Form F

Compound 1 Form F was prepared by slurrying Compound 1 in acetic acid:water (30:70, v/v) at ambient temperature for 7 days, as described in Table 3.1.

FIG. 17 provides an XRPD spectrum of Compound 1 Form F. The XRPD pattern of Form F exhibited well-resolved peaks with high intensity, characteristic of crystalline material. The XRPD pattern of Form F was successfully indexed indicating that the material is composed primarily or exclusively of a single crystalline phase. Based on the calculated volume from the indexing solution, the unit cell could accommodate one molecule of acetic acid per one molecule of Compound 1. XRPD data corresponding to Compound 1 Form F are summarized in Table 10.1.

TABLE 10.1

| °2θ | d-spacing (Å) |
|---|---|
| 6.93 ± 0.20 | 12.745 ± 0.367 |
| 8.19 ± 0.20 | 10.787 ± 0.263 |
| 8.50 ± 0.20 | 10.394 ± 0.244 |
| 9.25 ± 0.20 | 9.553 ± 0.206 |
| 11.21 ± 0.20 | 7.887 ± 0.140 |
| 12.83 ± 0.20 | 6.894 ± 0.107 |
| 13.60 ± 0.20 | 6.506 ± 0.095 |
| 14.53 ± 0.20 | 6.091 ± 0.083 |
| 15.30 ± 0.20 | 5.786 ± 0.075 |
| 15.81 ± 0.20 | 5.601 ± 0.070 |
| 16.44 ± 0.20 | 5.388 ± 0.065 |
| 17.08 ± 0.20 | 5.187 ± 0.060 |
| 17.35 ± 0.20 | 5.107 ± 0.058 |
| 17.54 ± 0.20 | 5.052 ± 0.057 |
| 18.05 ± 0.20 | 4.911 ± 0.054 |
| 18.43 ± 0.20 | 4.810 ± 0.052 |
| 18.64 ± 0.20 | 4.756 ± 0.051 |
| 20.16 ± 0.20 | 4.401 ± 0.043 |
| 20.32 ± 0.20 | 4.367 ± 0.043 |
| 20.86 ± 0.20 | 4.255 ± 0.040 |
| 21.32 ± 0.20 | 4.164 ± 0.039 |
| 21.69 ± 0.20 | 4.094 ± 0.037 |
| 22.14 ± 0.20 | 4.012 ± 0.036 |
| 22.33 ± 0.20 | 3.978 ± 0.035 |
| 22.69 ± 0.20 | 3.916 ± 0.034 |
| 22.83 ± 0.20 | 3.892 ± 0.034 |
| 23.23 ± 0.20 | 3.826 ± 0.032 |
| 23.76 ± 0.20 | 3.742 ± 0.031 |
| 24.03 ± 0.20 | 3.700 ± 0.030 |
| 24.60 ± 0.20 | 3.616 ± 0.029 |
| 25.20 ± 0.20 | 3.531 ± 0.028 |
| 25.84 ± 0.20 | 3.445 ± 0.026 |
| 27.06 ± 0.20 | 3.293 ± 0.024 |
| 27.73 ± 0.20 | 3.214 ± 0.023 |
| 28.24 ± 0.20 | 3.158 ± 0.022 |

The peak positions and integration values in the proton NMR spectrum of Form F were consistent with the chemical structures of Compound 1 and acetic acid in a 1:1 molar ratio. Traces of ethanol, as well as some unidentified impurities, were also present in the spectrum.

The DSC thermogram displayed a broad endotherm with peak maximum at 187.5° C. and an onset at 176.4° C., followed by an endotherm with peak maximum at 276.0° C. and an onset at 269.3° C. (FIG. 18). The TGA curve indicated no weight loss up to 130° C. (FIG. 18). A weight loss of 11.1% between 130° C. and 205° C. accompanied the broad endotherm and was most likely associated with the release of acetic acid. A weight loss of 11.1% corresponded to 1.09 moles of acetic acid.

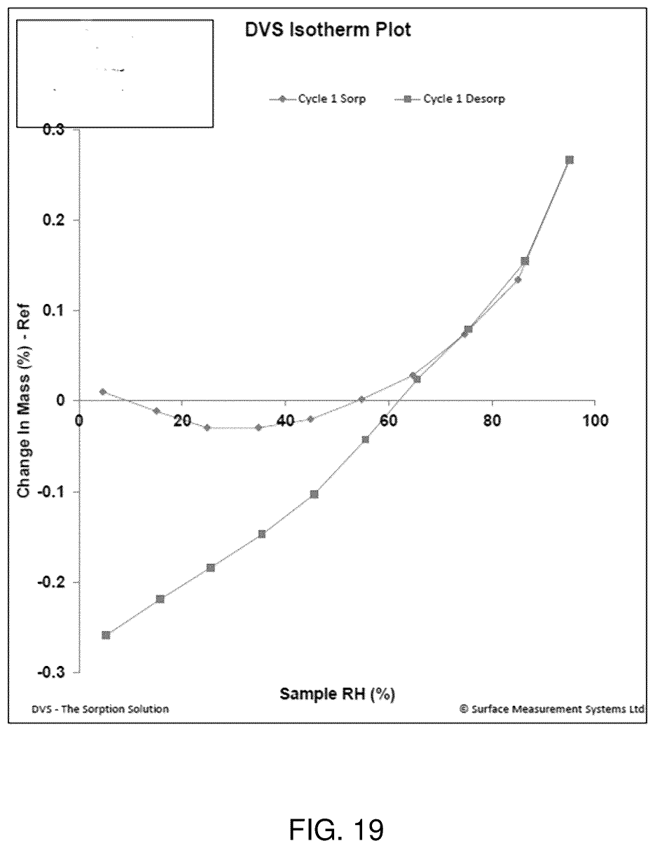

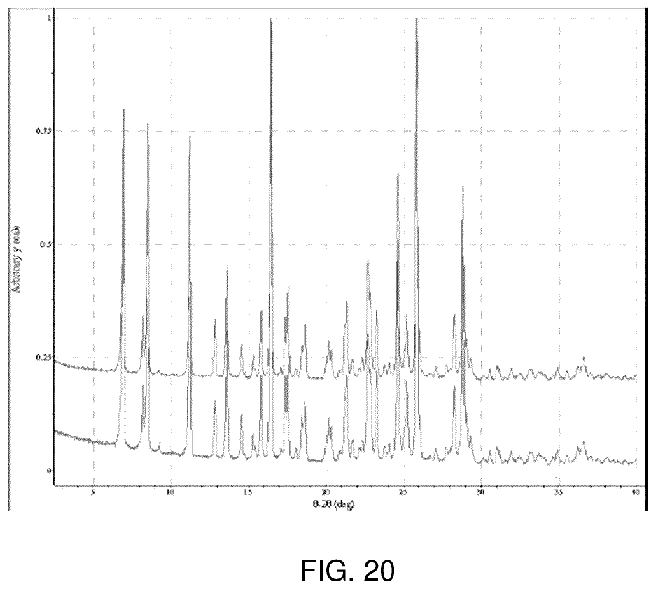

Form F was analyzed by DVS experiment (FIG. 19 and Table 10.2). An insignificant weight change was observed upon equilibration at 5% RH. A small weight gain of 0.3% during the absorption step at 5-95% RH and a 0.5% weight loss during the desorption step at 95-5% RH were indicative of non-hygroscopic material. The XRPD pattern of the post-DVS sample was consistent with Form F (FIG. 20), suggesting that Form F remained unchanged during the duration of the DVS experiment.

TABLE 10.2

| | | | | | Desorp | |
|---|---|---|---|---|---|---|
| | | | Sorp Mass | | Mass | |
| | Target | Sample | Change | Sample | Change | |
| | RH (%) | RH (%) | (%) | RH (%) | (%) | Hysteresis |
| Cycle 1 | 5.0 | 4.6 | 0.0100 | 5.3 | −0.2589 | |
| | 15.0 | 15.0 | −0.0114 | 15.7 | −0.2193 | −0.2079 |
| | 25.0 | 24.9 | −0.0296 | 25.5 | −0.1841 | −0.1545 |
| | 35.0 | 34.8 | −0.0296 | 35.5 | −0.1477 | −0.1181 |
| | 45.0 | 44.9 | −0.0202 | 45.6 | −0.1031 | −0.0829 |
| | 55.0 | 54.8 | 0.0018 | 55.5 | −0.0428 | −0.0446 |
| | 65.0 | 64.7 | 0.0282 | 65.5 | 0.0238 | −0.0044 |
| | 75.0 | 74.7 | 0.0741 | 75.5 | 0.0797 | 0.0057 |
| | 85.0 | 85.1 | 0.1344 | 86.4 | 0.1545 | 0.0201 |
| | 95.0 | 95.0 | 0.2669 | 95.0 | 0.2669 | |

Form F appeared to be practically insoluble in water (<1 mg/mL).

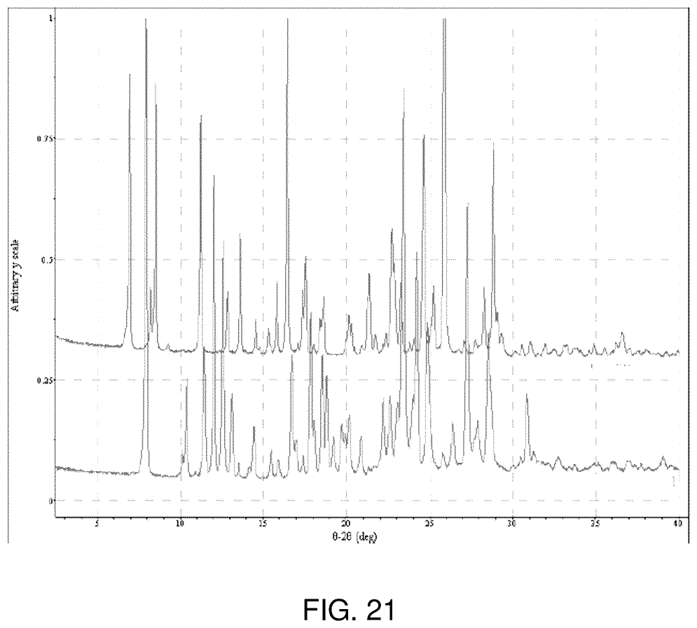

Form F converted completely to Form C via a slurry in water at ambient temperature for 3 days (FIG. 21), suggesting that Form F is more likely an acetic acid monosolvate rather than a monoacetate salt.

Based on the characterization data described above, Form F was assigned as an acetic acid monosolvate of Compound 1.

### Example 11. Compound 1 Form G

Compound 1 Form G was obtained from a vapor diffusion experiment of a solution of Compound 1 in MeOH:DCM (1:3, v/v) exposed to vapor of ethyl acetate, as described in Table 3.2.

FIG. 22 provides an XRPD spectrum of Compound 1 Form G. The XRPD pattern of Form G exhibited well-resolved peaks with high intensity, characteristic of crystalline material. The XRPD pattern of Form G was successfully indexed indicating that the material is composed primarily or exclusively of a single crystalline phase. Based on the calculated volume from the indexing solution, the unit cell could accommodate two molecules of EtOAc per one molecule of Compound 1. XRPD data corresponding to Compound 1 Form G are summarized in Table 11.1.

TABLE 11.1

| °2θ | d-spacing (Å) |
|---|---|
| 7.30 ± 0.20 | 12.100 ± 0.331 |
| 8.16 ± 0.20 | 10.826 ± 0.265 |
| 8.57 ± 0.20 | 10.309 ± 0.240 |
| 10.16 ± 0.20 | 8.699 ± 0.171 |
| 11.86 ± 0.20 | 7.456 ± 0.125 |
| 12.80 ± 0.20 | 6.910 ± 0.108 |
| 13.23 ± 0.20 | 6.687 ± 0.101 |
| 13.41 ± 0.20 | 6.597 ± 0.098 |
| 14.19 ± 0.20 | 6.236 ± 0.087 |
| 14.64 ± 0.20 | 6.046 ± 0.082 |
| 14.99 ± 0.20 | 5.905 ± 0.078 |
| 15.28 ± 0.20 | 5.794 ± 0.075 |
| 15.47 ± 0.20 | 5.723 ± 0.074 |
| 16.11 ± 0.20 | 5.497 ± 0.068 |
| 16.38 ± 0.20 | 5.407 ± 0.066 |
| 16.85 ± 0.20 | 5.257 ± 0.062 |
| 17.23 ± 0.20 | 5.142 ± 0.059 |
| 17.48 ± 0.20 | 5.069 ± 0.058 |
| 17.70 ± 0.20 | 5.007 ± 0.056 |
| 18.52 ± 0.20 | 4.787 ± 0.051 |
| 18.86 ± 0.20 | 4.701 ± 0.049 |
| 19.09 ± 0.20 | 4.645 ± 0.048 |
| 19.20 ± 0.20 | 4.619 ± 0.048 |
| 19.45 ± 0.20 | 4.560 ± 0.046 |
| 19.93 ± 0.20 | 4.451 ± 0.044 |
| 20.16 ± 0.20 | 4.401 ± 0.043 |
| 20.43 ± 0.20 | 4.344 ± 0.042 |
| 20.78 ± 0.20 | 4.271 ± 0.041 |
| 21.31 ± 0.20 | 4.166 ± 0.039 |
| 21.97 ± 0.20 | 4.042 ± 0.036 |
| 22.18 ± 0.20 | 4.005 ± 0.036 |
| 22.30 ± 0.20 | 3.983 ± 0.035 |
| 22.89 ± 0.20 | 3.882 ± 0.033 |
| 23.54 ± 0.20 | 3.776 ± 0.032 |
| 23.85 ± 0.20 | 3.728 ± 0.031 |

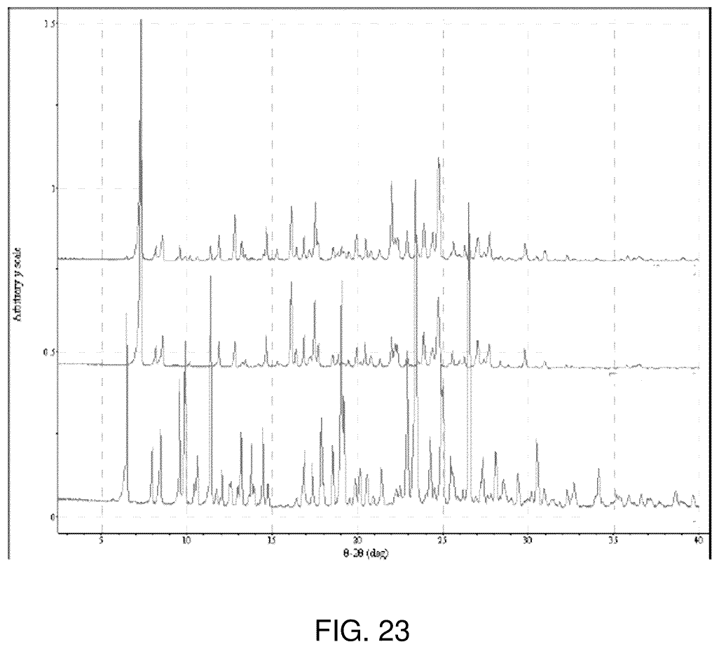

Proton NMR characterization was carried out on a sample comprising Form G and a minor amount of Form P (FIG. 23, Sample 3.5.4 from Table 3.5). The peak positions and integration values in the proton NMR spectrum were consistent with the chemical structure of Compound 1. Additionally, the following solvents were present: EtOAc in the amount of 1.08 moles (15.3%); MeOH in the amount of 0.21 mole (1.3%), and DCM in the amount of 0.19 mole (3.0%).

DSC/TGA characterization was carried out on a sample comprising Form G and a minor amount of Form P (FIG. 23, Sample 3.5.4 from Table 3.5). The DSC thermogram displayed a broad endotherm at 117.2° C., followed by a shoulder at 275.1° C., next to a large endotherm with peak maximum at 284.5° C. (FIG. 24). The TGA curve showed 13.2% weight loss between 47° C. and 180° C. that accompanied the broad endotherm and was associated with release of solvent from the sample, most likely with the release of EtOAc, though small amounts of MeOH and DCM were also present in the sample, as judged by NMR (FIG. 24).

Based on the characterization data described above, Form G was assigned as an ethyl acetate monosolvate of Compound 1.

### Example 12. Compound 1 Form H

Compound 1 Form H was obtained from a slurry of Compound 1 Form G in TAME at 50° C. for 4 days, followed by a slurry in TAME at ambient temperature for 6 days, as described in Table 3.7. Compound 1 Form H obtained in this method was disordered. Slurrying Compound 1 Form H in EtOH:H₂O (90:10, v/v) at ambient temperature for 3 days did not improve crystallinity.

FIG. 3B (second spectrum from top) provides an XRPD spectrum of Compound 1 Form H. The XRPD pattern of Form H exhibited broad peaks with high intensity, characteristic of crystalline material with disorder, which prevented its indexing. Compound 1. XRPD data corresponding to Compound 1 Form H are summarized in Table 12.1.

TABLE 12.1

| °2θ | d-spacing (Å) |
|---|---|
| 8.74 ± 0.20 | 10.115 ± 0.231 |
| 9.33 ± 0.20 | 9.467 ± 0.202 |
| 9.63 ± 0.20 | 9.176 ± 0.190 |
| 10.97 ± 0.20 | 8.057 ± 0.146 |
| 14.60 ± 0.20 | 6.062 ± 0.083 |
| 15.23 ± 0.20 | 5.812 ± 0.076 |
| 16.53 ± 0.20 | 5.358 ± 0.064 |
| 17.47 ± 0.20 | 5.073 ± 0.058 |
| 18.10 ± 0.20 | 4.898 ± 0.054 |
| 18.78 ± 0.20 | 4.720 ± 0.050 |
| 19.35 ± 0.20 | 4.584 ± 0.047 |
| 20.36 ± 0.20 | 4.358 ± 0.042 |
| 22.17 ± 0.20 | 4.006 ± 0.036 |
| 22.40 ± 0.20 | 3.965 ± 0.035 |
| 23.50 ± 0.20 | 3.782 ± 0.032 |
| 24.02 ± 0.20 | 3.702 ± 0.030 |
| 26.05 ± 0.20 | 3.417 ± 0.026 |
| 26.38 ± 0.20 | 3.375 ± 0.025 |
| 27.64 ± 0.20 | 3.225 ± 0.023 |
| 29.45 ± 0.20 | 3.031 ± 0.020 |
| 29.84 ± 0.20 | 2.992 ± 0.020 |
| 31.07 ± 0.20 | 2.876 ± 0.018 |

The peak positions and integration values in the proton NMR spectrum of Compound 1 Form H were consistent with the chemical structure of Compound 1. Traces of ethanol and TAME, as well as some unidentified impurity were also present in the spectrum.

The DSC thermogram demonstrated an endotherm with peak maximum at 272.2° C. and an onset at 264.9° C. (FIG. 25). The TGA curve indicated insignificant weight loss of 0.3% between 42° C. and 160° C., most likely associated with the release of residual solvents (FIG. 25).

Based on characterization data described above, Form H was assigned as an anhydrous/non-solvated form of Compound 1. Form H was found to be a metastable anhydrous form with respect to Form B from ambient temperature to 65° C.

### Example 13. Compound 1 Form I

Compound 1 Form I was obtained in mixtures with Form B and/or Form H, either by heating Compound 1 Form A, Form D, or Form G (as described in Table 3.6) or by solvent-based slurry of Compound 1 Form D or Form G in bulky solvents such as Bu₂O, CPME, or TAME (Table 3.7).

FIG. 3B (third spectrum from top) provides an XRPD spectrum of Compound 1 Form I, as a mixture with Compound 1 Form H, obtained by heating Compound 1 Form D to 195° C.

Based on various methods used to prepare Compound 1 Form I, it was assigned to be most likely an anhydrous form of Compound 1.

A mixture of Compound 1 Form H and Compound 1 Form I was utilized in interconversion slurry experiments with Form B. Compound 1 Form I was found to be a metastable form with respect to Form B from ambient temperature to 65° C.

### Example 14. Compound 1 Form J

Compound 1 Form J was obtained from a slurry of Compound 1 Form G in MTBE at ambient temperature for 6 days, as described in Table 3.7.

FIG. 26 provides an XRPD spectrum of Compound 1 Form J. The XRPD pattern of Form J exhibited well-resolved peaks with high intensity, characteristic of crystalline material. The XRPD pattern of Form J was successfully indexed indicating that the material is composed primarily or exclusively of a single crystalline phase. Based on the calculated volume from the indexing solution, the unit cell could accommodate up to two molecules of MTBE per one molecule of Compound 1. Compound 1. XRPD data corresponding to Compound 1 Form J are summarized in Table 14.1.

TABLE 14.1

| °2θ | d-spacing (Å) |
|---|---|
| 7.00 ± 0.20 | 12.618 ± 0.360 |
| 8.35 ± 0.20 | 10.581 ± 0.253 |
| 10.09 ± 0.20 | 8.760 ± 0.173 |
| 10.99 ± 0.20 | 8.044 ± 0.146 |
| 11.27 ± 0.20 | 7.845 ± 0.139 |
| 11.79 ± 0.20 | 7.500 ± 0.127 |
| 12.02 ± 0.20 | 7.357 ± 0.122 |
| 12.97 ± 0.20 | 6.820 ± 0.105 |
| 13.79 ± 0.20 | 6.416 ± 0.093 |
| 14.06 ± 0.20 | 6.294 ± 0.089 |
| 14.44 ± 0.20 | 6.129 ± 0.084 |
| 14.82 ± 0.20 | 5.973 ± 0.080 |
| 15.10 ± 0.20 | 5.863 ± 0.077 |
| 16.19 ± 0.20 | 5.470 ± 0.067 |
| 16.52 ± 0.20 | 5.362 ± 0.064 |
| 16.92 ± 0.20 | 5.236 ± 0.061 |
| 17.16 ± 0.20 | 5.163 ± 0.060 |
| 17.46 ± 0.20 | 5.075 ± 0.058 |
| 17.73 ± 0.20 | 4.998 ± 0.056 |
| 17.91 ± 0.20 | 4.949 ± 0.055 |
| 18.66 ± 0.20 | 4.751 ± 0.050 |
| 19.14 ± 0.20 | 4.633 ± 0.048 |
| 19.61 ± 0.20 | 4.523 ± 0.046 |
| 19.86 ± 0.20 | 4.467 ± 0.045 |
| 20.28 ± 0.20 | 4.375 ± 0.043 |
| 20.86 ± 0.20 | 4.255 ± 0.040 |
| 21.16 ± 0.20 | 4.195 ± 0.039 |
| 21.58 ± 0.20 | 4.115 ± 0.038 |
| 21.81 ± 0.20 | 4.072 ± 0.037 |
| 22.10 ± 0.20 | 4.019 ± 0.036 |
| 22.65 ± 0.20 | 3.923 ± 0.034 |
| 23.05 ± 0.20 | 3.855 ± 0.033 |
| 23.59 ± 0.20 | 3.768 ± 0.031 |
| 23.94 ± 0.20 | 3.714 ± 0.031 |
| 24.30 ± 0.20 | 3.660 ± 0.030 |
| 24.98 ± 0.20 | 3.562 ± 0.028 |
| 26.13 ± 0.20 | 3.408 ± 0.026 |
| 26.73 ± 0.20 | 3.332 ± 0.024 |
| 27.14 ± 0.20 | 3.283 ± 0.024 |
| 27.44 ± 0.20 | 3.248 ± 0.023 |
| 27.82 ± 0.20 | 3.204 ± 0.023 |
| 28.31 ± 0.20 | 3.150 ± 0.022 |
| 29.08 ± 0.20 | 3.068 ± 0.021 |

The peak positions and integration values in the proton NMR spectrum of Form J were consistent with the chemical structure of Compound 1. Singlets at 1.10 ppm and 3.08 ppm in the spectrum were indicative of ˜0.74 mole of MTBE present in the material.

Based on the characterization data described above, Compound 1 Form J was assigned as a MTBE monosolvate of Compound 1.

### Example 15. Compound 1 Form K

Compound 1 Form K was obtained from a slurry of Compound 1 Form D in Bu₂O at 35° C. for 5 days, as described in Table 3.7.

FIG. 27 provides an XRPD spectrum of Compound 1 Form K. The XRPD pattern of Form K exhibited well-resolved peaks with high intensity, characteristic of crystalline material. The XRPD pattern of Form K was successfully indexed indicating that the material is composed primarily or exclusively of a single crystalline phase. Based on the calculated volume from the indexing solution, the unit cell could accommodate one molecule of Bu₂O per one molecule of Compound 1. XRPD data corresponding to Compound 1 Form K are summarized in Table 15.1.

TABLE 15.1

| °2θ | d-spacing (Å) |
|---|---|
| 7.21 ± 0.20 | 12.251 ± 0.339 |
| 8.35 ± 0.20 | 10.581 ± 0.253 |
| 10.22 ± 0.20 | 8.648 ± 0.169 |
| 11.77 ± 0.20 | 7.513 ± 0.127 |
| 12.50 ± 0.20 | 7.076 ± 0.113 |
| 13.18 ± 0.20 | 6.712 ± 0.101 |
| 14.47 ± 0.20 | 6.116 ± 0.084 |
| 14.98 ± 0.20 | 5.909 ± 0.078 |
| 16.17 ± 0.20 | 5.477 ± 0.067 |
| 16.67 ± 0.20 | 5.314 ± 0.063 |
| 17.13 ± 0.20 | 5.172 ± 0.060 |
| 17.25 ± 0.20 | 5.136 ± 0.059 |
| 17.80 ± 0.20 | 4.979 ± 0.055 |
| 18.13 ± 0.20 | 4.889 ± 0.053 |
| 18.73 ± 0.20 | 4.734 ± 0.050 |
| 19.20 ± 0.20 | 4.619 ± 0.048 |
| 19.51 ± 0.20 | 4.546 ± 0.046 |
| 20.07 ± 0.20 | 4.421 ± 0.044 |
| 20.35 ± 0.20 | 4.360 ± 0.042 |
| 20.55 ± 0.20 | 4.318 ± 0.042 |
| 20.79 ± 0.20 | 4.269 ± 0.041 |
| 21.37 ± 0.20 | 4.155 ± 0.038 |
| 21.73 ± 0.20 | 4.087 ± 0.037 |
| 22.15 ± 0.20 | 4.010 ± 0.036 |
| 22.62 ± 0.20 | 3.928 ± 0.034 |
| 23.38 ± 0.20 | 3.802 ± 0.032 |
| 23.70 ± 0.20 | 3.751 ± 0.031 |
| 24.08 ± 0.20 | 3.693 ± 0.030 |
| 24.47 ± 0.20 | 3.635 ± 0.029 |
| 25.09 ± 0.20 | 3.546 ± 0.028 |
| 25.61 ± 0.20 | 3.476 ± 0.027 |
| 25.93 ± 0.20 | 3.433 ± 0.026 |
| 26.58 ± 0.20 | 3.351 ± 0.025 |
| 26.91 ± 0.20 | 3.311 ± 0.024 |
| 27.58 ± 0.20 | 3.232 ± 0.023 |
| 27.99 ± 0.20 | 3.185 ± 0.022 |
| 28.98 ± 0.20 | 3.078 ± 0.021 |
| 29.50 ± 0.20 | 3.025 ± 0.020 |

The peak positions and integration values in the proton NMR spectrum of Form K were consistent with the chemical structure of Compound 1. A triplet at 0.86 ppm and multiplets at 1.27-1.48 ppm in the spectrum were indicative of ˜ 0.8 mole of Bu₂O present in the sample.

Based on the characterization data described above, Compound 1 Form K was assigned as a Bu₂O monosolvate of Compound 1.

### Example 16. Compound 1 Form L

Compound 1 Form L was obtained from a slurry in DCM at ambient temperature for days, as described in Table 3.1.

FIG. 28 provides an XRPD spectrum of Compound 1 Form L. The XRPD pattern of Form L exhibited well-resolved peaks with high intensity, characteristic of crystalline material. The XRPD pattern of Form L was successfully indexed indicating that the material is composed primarily or exclusively of a single crystalline phase. Based on the calculated volume from the indexing solution, the unit cell could accommodate up to three molecules of DCM per one molecule of Compound 1. XRPD data corresponding to Compound 1 Form L are summarized in Table 16.1.

TABLE 16.1

| °2θ | d-spacing (Å) |
|---|---|
| 7.30 ± 0.20 | 12.100 ± 0.331 |
| 8.34 ± 0.20 | 10.593 ± 0.254 |
| 8.43 ± 0.20 | 10.480 ± 0.248 |
| 9.34 ± 0.20 | 9.461 ± 0.202 |
| 10.32 ± 0.20 | 8.565 ± 0.166 |
| 11.88 ± 0.20 | 7.443 ± 0.125 |
| 12.65 ± 0.20 | 6.992 ± 0.110 |
| 13.14 ± 0.20 | 6.732 ± 0.102 |
| 13.44 ± 0.20 | 6.583 ± 0.098 |
| 14.01 ± 0.20 | 6.316 ± 0.090 |
| 14.62 ± 0.20 | 6.054 ± 0.082 |
| 15.43 ± 0.20 | 5.738 ± 0.074 |
| 16.21 ± 0.20 | 5.464 ± 0.067 |
| 16.45 ± 0.20 | 5.384 ± 0.065 |
| 16.61 ± 0.20 | 5.333 ± 0.064 |
| 16.93 ± 0.20 | 5.233 ± 0.061 |
| 17.10 ± 0.20 | 5.181 ± 0.060 |
| 17.96 ± 0.20 | 4.935 ± 0.055 |
| 18.49 ± 0.20 | 4.795 ± 0.051 |
| 18.77 ± 0.20 | 4.724 ± 0.050 |
| 18.89 ± 0.20 | 4.694 ± 0.049 |
| 19.54 ± 0.20 | 4.540 ± 0.046 |
| 19.92 ± 0.20 | 4.454 ± 0.044 |
| 20.75 ± 0.20 | 4.277 ± 0.041 |
| 21.03 ± 0.20 | 4.221 ± 0.040 |
| 21.28 ± 0.20 | 4.172 ± 0.039 |
| 21.42 ± 0.20 | 4.145 ± 0.038 |
| 21.70 ± 0.20 | 4.092 ± 0.037 |
| 21.92 ± 0.20 | 4.052 ± 0.037 |
| 22.21 ± 0.20 | 3.999 ± 0.036 |
| 22.82 ± 0.20 | 3.894 ± 0.034 |
| 23.96 ± 0.20 | 3.711 ± 0.031 |
| 24.47 ± 0.20 | 3.635 ± 0.029 |
| 24.75 ± 0.20 | 3.594 ± 0.029 |
| 25.21 ± 0.20 | 3.530 ± 0.028 |
| 25.64 ± 0.20 | 3.472 ± 0.027 |
| 25.93 ± 0.20 | 3.433 ± 0.026 |
| 26.10 ± 0.20 | 3.411 ± 0.026 |

The peak positions and integration values in the proton NMR spectrum of Form L were consistent with the chemical structure of Compound 1. The presence of DCM (0.85 mole, 12.0%) was indicated by a singlet at 5.76 ppm. Traces of ethanol, as well as some unidentified impurity, were also observed in the spectrum.

The DSC thermogram displayed a broad endotherm with peak maximum at 90.3° C., followed by an exotherm with peak maximum at 174.8° C. and an onset at 162.8° C. (FIG. 29). An endotherm with peak maximum at 279.7° C. and an onset at 274.5° C. was also observed in the DSC thermogram. The TGA curve indicated 8.5% weight loss between 46° C. and 180° C. that accompanied the broad endotherm and corresponded to 0.6 mole of DCM (FIG. 29).

Based on the characterization data described above, Compound 1 Form L was assigned as a DCM monosolvate of Compound 1.

### Example 17. Compound 1 Form M

Compound 1 Form M was obtained by slurrying Compound 1 Form F in CPME at 50° C. for 3 days, as described in Table 3.7.

FIG. 30 provides an XRPD spectrum of Compound 1 Form M. The XRPD pattern of Form M exhibited well-resolved peaks with high intensity, characteristic of crystalline material. The XRPD pattern of Form M was successfully indexed indicating that the material is composed primarily or exclusively of a single crystalline phase. Based on the calculated volume from the indexing solution, the unit cell could accommodate one molecule of CPME per one molecule of Compound 1. XRPD data corresponding to Compound 1 Form M are summarized in Table 17.1.

TABLE 17.1

| °2θ | d-spacing (Å) |
|---|---|
| 7.13 ± 0.20 | 12.388 ± 0.347 |
| 8.30 ± 0.20 | 10.644 ± 0.256 |
| 8.49 ± 0.20 | 10.406 ± 0.245 |
| 10.13 ± 0.20 | 8.725 ± 0.172 |
| 11.10 ± 0.20 | 7.965 ± 0.143 |
| 11.89 ± 0.20 | 7.437 ± 0.125 |
| 12.33 ± 0.20 | 7.173 ± 0.116 |
| 13.23 ± 0.20 | 6.687 ± 0.101 |
| 14.29 ± 0.20 | 6.193 ± 0.086 |
| 14.43 ± 0.20 | 6.133 ± 0.085 |
| 15.02 ± 0.20 | 5.894 ± 0.078 |
| 16.21 ± 0.20 | 5.464 ± 0.067 |
| 16.67 ± 0.20 | 5.314 ± 0.063 |
| 17.06 ± 0.20 | 5.193 ± 0.060 |
| 17.63 ± 0.20 | 5.027 ± 0.057 |
| 18.50 ± 0.20 | 4.792 ± 0.051 |
| 18.73 ± 0.20 | 4.734 ± 0.050 |
| 19.00 ± 0.20 | 4.667 ± 0.049 |
| 19.62 ± 0.20 | 4.521 ± 0.046 |
| 19.89 ± 0.20 | 4.460 ± 0.044 |
| 20.35 ± 0.20 | 4.360 ± 0.042 |
| 20.68 ± 0.20 | 4.292 ± 0.041 |
| 21.16 ± 0.20 | 4.195 ± 0.039 |
| 21.52 ± 0.20 | 4.126 ± 0.038 |
| 22.07 ± 0.20 | 4.024 ± 0.036 |
| 22.32 ± 0.20 | 3.980 ± 0.035 |
| 23.36 ± 0.20 | 3.805 ± 0.032 |
| 23.93 ± 0.20 | 3.716 ± 0.031 |
| 24.22 ± 0.20 | 3.672 ± 0.030 |
| 24.63 ± 0.20 | 3.612 ± 0.029 |
| 25.28 ± 0.20 | 3.520 ± 0.027 |
| 25.45 ± 0.20 | 3.497 ± 0.027 |
| 25.73 ± 0.20 | 3.460 ± 0.026 |
| 25.95 ± 0.20 | 3.431 ± 0.026 |
| 26.30 ± 0.20 | 3.386 ± 0.025 |
| 26.66 ± 0.20 | 3.341 ± 0.025 |
| 27.08 ± 0.20 | 3.290 ± 0.024 |
| 27.58 ± 0.20 | 3.232 ± 0.023 |

The peak positions and integration values in the proton NMR spectrum of Form M were consistent with the chemical structure of Compound 1. Multiplets at 1.43-166 ppm, a singlet at 3.26 ppm and a multiplet at 3.72-3.78 ppm in the spectrum were indicative of ˜ 0.93 mole of CPME present in the sample.

Based on the characterization data described above, Compound 1 Form M was assigned as a CPME monosolvate of Compound 1.

### Example 18. Compound 1 Form N

Compound 1 Form N was obtained by slurrying Compound 1 in NMP at ambient temperature for 5 days, as described in Table 3.1.

FIG. 31 provides an XRPD spectrum of Compound 1 Form N. The XRPD pattern of Form N exhibited well-resolved peaks with high intensity, characteristic of crystalline material. The XRPD pattern of Form N was successfully indexed indicating that the material is composed primarily or exclusively of a single crystalline phase. Based on the calculated volume from the indexing solution, the unit cell could accommodate two molecules of NMP per one molecule of Compound 1. XRPD data corresponding to Compound 1 Form N are summarized in Table 18.1.

TABLE 18.1

| °2θ | d-spacing (Å) |
|---|---|
| 7.09 ± 0.20 | 12.458 ± 0.351 |
| 8.15 ± 0.20 | 10.840 ± 0.266 |
| 8.25 ± 0.20 | 10.709 ± 0.259 |
| 10.08 ± 0.20 | 8.768 ± 0.174 |
| 10.89 ± 0.20 | 8.118 ± 0.149 |
| 11.57 ± 0.20 | 7.642 ± 0.132 |
| 12.28 ± 0.20 | 7.202 ± 0.117 |
| 12.91 ± 0.20 | 6.852 ± 0.106 |
| 13.06 ± 0.20 | 6.773 ± 0.103 |
| 13.69 ± 0.20 | 6.463 ± 0.094 |
| 14.04 ± 0.20 | 6.303 ± 0.089 |
| 14.22 ± 0.20 | 6.223 ± 0.087 |
| 14.63 ± 0.20 | 6.050 ± 0.082 |
| 14.87 ± 0.20 | 5.953 ± 0.080 |
| 15.90 ± 0.20 | 5.569 ± 0.070 |
| 16.00 ± 0.20 | 5.535 ± 0.069 |
| 16.31 ± 0.20 | 5.430 ± 0.066 |
| 16.52 ± 0.20 | 5.362 ± 0.064 |
| 16.99 ± 0.20 | 5.214 ± 0.061 |
| 17.56 ± 0.20 | 5.046 ± 0.057 |
| 17.74 ± 0.20 | 4.996 ± 0.056 |
| 17.94 ± 0.20 | 4.940 ± 0.055 |
| 18.34 ± 0.20 | 4.834 ± 0.052 |
| 18.49 ± 0.20 | 4.795 ± 0.051 |
| 18.78 ± 0.20 | 4.721 ± 0.050 |
| 18.97 ± 0.20 | 4.674 ± 0.049 |
| 19.15 ± 0.20 | 4.631 ± 0.048 |
| 19.33 ± 0.20 | 4.588 ± 0.047 |
| 19.53 ± 0.20 | 4.542 ± 0.046 |
| 19.88 ± 0.20 | 4.462 ± 0.044 |
| 20.24 ± 0.20 | 4.384 ± 0.043 |
| 20.57 ± 0.20 | 4.314 ± 0.041 |
| 20.83 ± 0.20 | 4.261 ± 0.040 |
| 21.30 ± 0.20 | 4.168 ± 0.039 |
| 21.56 ± 0.20 | 4.118 ± 0.038 |
| 21.93 ± 0.20 | 4.050 ± 0.036 |
| 22.83 ± 0.20 | 3.892 ± 0.034 |
| 23.25 ± 0.20 | 3.823 ± 0.032 |
| 23.51 ± 0.20 | 3.781 ± 0.032 |
| 23.82 ± 0.20 | 3.733 ± 0.031 |
| 24.01 ± 0.20 | 3.703 ± 0.030 |
| 24.08 ± 0.20 | 3.693 ± 0.030 |
| 24.61 ± 0.20 | 3.614 ± 0.029 |
| 24.82 ± 0.20 | 3.584 ± 0.028 |
| 25.16 ± 0.20 | 3.537 ± 0.028 |
| 25.58 ± 0.20 | 3.480 ± 0.027 |
| 25.76 ± 0.20 | 3.456 ± 0.026 |
| 25.99 ± 0.20 | 3.426 ± 0.026 |
| 26.21 ± 0.20 | 3.398 ± 0.025 |
| 26.42 ± 0.20 | 3.371 ± 0.025 |
| 26.54 ± 0.20 | 3.356 ± 0.025 |
| 26.86 ± 0.20 | 3.317 ± 0.024 |
| 27.10 ± 0.20 | 3.288 ± 0.024 |
| 27.58 ± 0.20 | 3.232 ± 0.023 |
| 27.81 ± 0.20 | 3.206 ± 0.023 |
| 28.17 ± 0.20 | 3.165 ± 0.022 |
| 28.36 ± 0.20 | 3.145 ± 0.022 |
| 28.53 ± 0.20 | 3.126 ± 0.021 |
| 28.89 ± 0.20 | 3.088 ± 0.021 |
| 29.08 ± 0.20 | 3.068 ± 0.021 |

Based on the characterization data described above, Compound 1 Form N was assigned as an NMP solvate of Compound 1.

### Example 19. Compound 1 Form O

Compound 1 Form O was obtained by fast evaporation of a solution of Compound 1 in TFE:H₂O (95:5) at ambient conditions, as described in Table 3.2.

FIG. 32 provides an XRPD spectrum of Compound 1 Form O. The XRPD pattern of Form O exhibited well-resolved peaks with high intensity, characteristic of crystalline material. The XRPD pattern of Form O was successfully indexed indicating that the material is composed primarily or exclusively of a single crystalline phase. Based on the calculated volume from the indexing solution, the unit cell could accommodate two molecule of THE per one molecule of Compound 1. XRPD data corresponding to Compound 1 Form O are summarized in Table 19.1.

TABLE 19.1

| °2θ | d-spacing (Å) |
|---|---|
| 7.44 ± 0.20 | 11.873 ± 0.319 |
| 8.18 ± 0.20 | 10.800 ± 0.264 |
| 8.34 ± 0.20 | 10.593 ± 0.254 |
| 9.19 ± 0.20 | 9.615 ± 0.209 |
| 10.38 ± 0.20 | 8.515 ± 0.164 |
| 11.70 ± 0.20 | 7.558 ± 0.129 |
| 13.56 ± 0.20 | 6.525 ± 0.096 |
| 13.99 ± 0.20 | 6.325 ± 0.090 |
| 14.91 ± 0.20 | 5.937 ± 0.079 |
| 15.38 ± 0.20 | 5.757 ± 0.074 |
| 15.95 ± 0.20 | 5.552 ± 0.069 |
| 16.49 ± 0.20 | 5.371 ± 0.065 |
| 16.75 ± 0.20 | 5.289 ± 0.063 |
| 16.94 ± 0.20 | 5.230 ± 0.061 |
| 17.57 ± 0.20 | 5.044 ± 0.057 |
| 17.87 ± 0.20 | 4.960 ± 0.055 |
| 18.46 ± 0.20 | 4.802 ± 0.052 |
| 19.03 ± 0.20 | 4.660 ± 0.049 |
| 19.91 ± 0.20 | 4.456 ± 0.044 |
| 20.12 ± 0.20 | 4.410 ± 0.043 |
| 20.85 ± 0.20 | 4.257 ± 0.040 |
| 21.24 ± 0.20 | 4.180 ± 0.039 |
| 21.47 ± 0.20 | 4.135 ± 0.038 |
| 21.82 ± 0.20 | 4.070 ± 0.037 |
| 22.05 ± 0.20 | 4.028 ± 0.036 |
| 22.46 ± 0.20 | 3.955 ± 0.035 |
| 22.83 ± 0.20 | 3.892 ± 0.034 |
| 23.44 ± 0.20 | 3.792 ± 0.032 |
| 24.01 ± 0.20 | 3.703 ± 0.030 |
| 24.43 ± 0.20 | 3.641 ± 0.029 |
| 24.77 ± 0.20 | 3.591 ± 0.029 |
| 25.18 ± 0.20 | 3.534 ± 0.028 |
| 26.01 ± 0.20 | 3.423 ± 0.026 |
| 26.15 ± 0.20 | 3.405 ± 0.026 |
| 26.41 ± 0.20 | 3.372 ± 0.025 |
| 27.01 ± 0.20 | 3.298 ± 0.024 |
| 27.19 ± 0.20 | 3.277 ± 0.024 |
| 27.48 ± 0.20 | 3.243 ± 0.023 |
| 27.83 ± 0.20 | 3.203 ± 0.023 |
| 28.15 ± 0.20 | 3.167 ± 0.022 |
| 28.68 ± 0.20 | 3.110 ± 0.021 |
| 28.96 ± 0.20 | 3.081 ± 0.021 |
| 29.69 ± 0.20 | 3.006 ± 0.020 |
| 30.28 ± 0.20 | 2.949 ± 0.019 |
| 30.67 ± 0.20 | 2.913 ± 0.019 |

Based on the characterization data described above, Compound 1 Form O was assigned as a TFE solvate of Compound 1.

### Example 20. Compound 1 Form P and Form Q

A mixture of Compound 1 Form P and Compound 1 Form Q were obtained from a vapor diffusion experiment of a solution of Compound 1 in MeOH:DCM (1:3, v/v) exposed to a vapor of ethyl acetate saturated with water, as described in Table 3.2. FIG. 3C (fourth spectrum from top) provides an XRPD spectrum of the resulting mixture of Compound 1 Form P and Compound 1 Form Q.

A small amount of Compound 1 Form P was also observed in certain samples of Compound 1 Form G, as described above (FIG. 23, Sample 3.5.4 from Table 3.5).

### Example 21. Amorphous Compound 1

Amorphous Compound 1 was prepared using the CESS® process described in J. Pessi, et al., Controlled Expansion of Supercritical Solution: A Robust Method to Produce Pure Drug Nanoparticles With Narrow Size-Distribution, J. Pharm. Sci., 2016, 105, 2293-97. Briefly, Compound 1 was loaded into a thermostatic pressure vessel and was dissolved in supercritical CO₂ at a specified temperature and pressure. Next, the solution was driven from the solubilization vessel through a nozzle into a collection vessel under thermodynamic flow control. The temperature of the mixture before the nozzle was regulated during the process. During expansion at the nozzle, the pressure and temperature of supercritical CO₂ decrease, leading to formation of solid CO₂ (dry ice flakes), which entrapped Compound 1 nanoparticles. Table 21.1 summarizes the parameters used in these experiments:

TABLE 21.1

| | | Solubilization | Nanonization | |
|---|---|---|---|---|
| Expt. | Pressure | Temperature, | Temperature, | Throughput |
| No. | (bar) | Tₛ (° C.) | Tₙ (° C.) | (mg/h) |
| 1 | 300 | 58 | 60 | — |
| 2 | 450 | 62 | 60 | — |
| 3 | 600 | 61 | 60 | 5 |
| 4 | 300 | 77 | 76 | 33 |
| 5 | 450 | 78 | 76 | 7 |
| 6 | 600 | 78 | 76 | 14 |
| 7 | 300 | 99 | 92 | 7 |
| 8 | 450 | 99 | 92 | 18 |
| 9 | 600 | 98 | 92 | 103 |

### Scanning Electron Microscopy (SEM)

Scanning electron microscopy (SEM) images from bulk and nanoformed material were taken with a Zeiss Sigma 300 VP Field Emission SEM to evaluate the particle size and shape. Briefly, the sample was placed onto an SEM stub partially covered with carbon tape and sputtered with Pt (typical coating thickness of 5-8 nm as measured by a QCM sensor and assuming a density of 21.45 g/cm³ for Pt). 10 to 15 kV acceleration voltages were used. A representative image with the highest number of visually identifiable individual particles was chosen for further size distribution analysis. Particles were counted from images and classified through the attribution of measurable ellipsoid shapes. For nanoformed Compound 1, the magnifications that allowed this classification were between ×10.000 and ×25.000. Each histogram was based on 60 or more randomly chosen particles counted. The evaluated statistics for SEM imaging are:
D50: Median particle size (in nm), i.e., in the case of ellipsoids, is the geometric average of the length of the two main axes that define the ellipsoid (xy-coordinate). The value is the same as the diameter for a circle that would have the same total area.
Span: This value gives a statistic on the width of the particle size distribution. The span is defined as (D90−D10)/D50. D10 refers to the 10-percentile mark, i.e., 10% of all the particles are smaller than this value (and conversely, 90% of the particles are larger than this value). D90 is defined similarly, i.e., 90% of all particles are smaller than this value. The span value is normalized to the D50 value.
Min and Max: correspond to the smallest and largest particle size (D, diameter) found in the analyses, respectively.

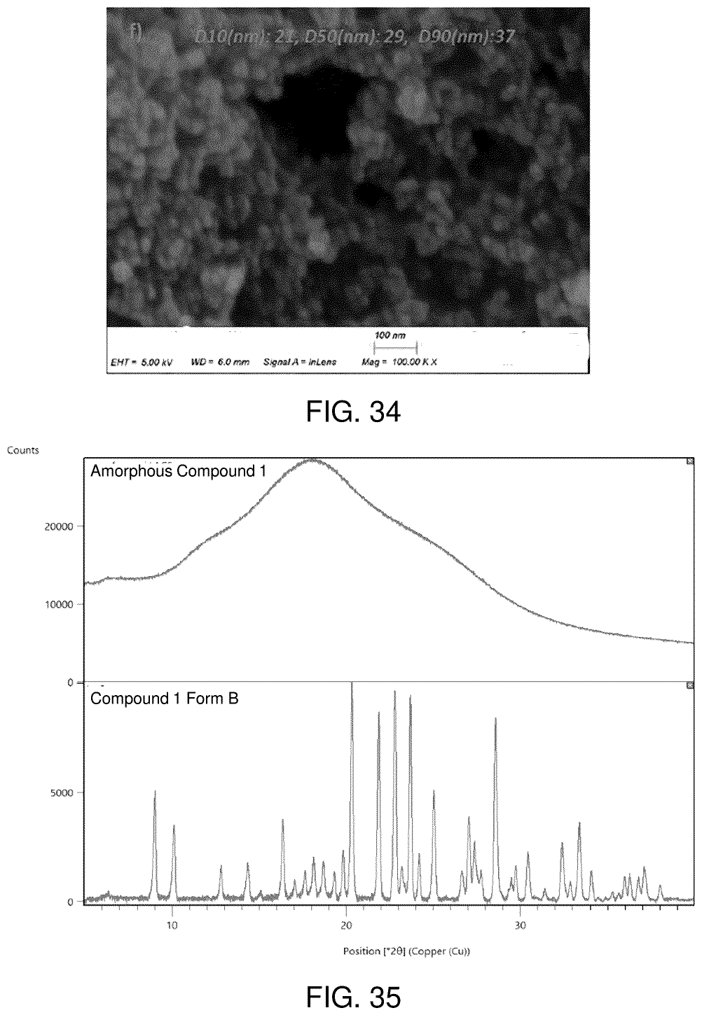

The material obtained from the experiments in Table 21.1 were analyzed using SEM. The experiments generally resulted in spherical particles. Particles produced at low pressure (experiments 4 and 7) displayed relatively large sizes with D50 of 68 nm and 139 nm, respectively. At high pressure (experiments 6 and 9), the resulting particles were smaller with D50 of 32 and 29 nm, respectively. At intermediate pressure (experiments 5 and 8), the resulting particles had D50 of 40 and 34 nm, respectively. FIG. 34 is a SEM image of the material obtained from experiment 9.

### XRPD

XRPD analyses were carried out on a Malvern PANalytical Empyrean X-ray diffractometer equipped with a multicore optics and solid-state PIXcel3D detector. Powder samples were loaded in a standard backloading sample holder sealed with Kapton tape. Cu Kα (1.54 Å) radiation was generated using a voltage of 45 kV and a current of 40 mA. Measurements were performed from 5-40 [°2θ] with a step size of 0.007 [°2θ] and a scanning step time of 59.6 s. The diffractograms were plotted using OriginLab 2021 software.

XRPD analysis performed on the material obtained from the experiments described in Table 21.1 indicated that it was amorphous. A representative XRPD spectrum is shown in FIG. 35, relative to Compound 1 Form B.

### DSC

DSC measurements were performed using the Discovery DSC 250 instrument under dry N₂(g) purge. The samples, typically 3-5 mg, were placed in a TO low-mass pan (aluminum), and the pan was closed with a proper lid. The routine used was the first cycle-heating up to 285° C., at a heating rate of 10° C./min; cooling step down to −40° C. at 50° C./min. The second cycle-heating up to 285° C. with a heating rate of 3° C./min and temperature modulation of 0.5° C. at every 60 seconds. The thermal events were depicted from the second cycle. The nanoformed material was measured with similar settings as that of the second cycle heating program.

The thermal behavior of the nanoformed material was determined using DSC. The DSC thermogram (FIG. 36) indicated that the amorphous nanoparticles had an endotherm between 10-90° C., which is typical of evaporation of absorbed moisture. The major thermal phenomenon is the glass transition, whose temperature was determined from the reversing heat flow signal and recorded at 139.8° C.

### Example 22. Biological Characterization of Compound 1

### JAK2 Binding Assay

JAK2 (JH1domain-catalytic, Y1007F, Y1008F) kinase was expressed as N-terminal fusion to the DNA binding domain of NFkB in transiently transfected HEK293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mmol/L DTT) to remove unbound ligand and to reduce nonspecific phage binding. Binding reactions were assembled by combining kinases, liganded affinity beads, and test compounds in 1× binding buffer (1×PBS, 0.05% Tween 20, 0.1% BSA, 1 mmol/L DTT). Test compound was prepared as 111× stocks in 100% DMSO and directly diluted into the assay wells. All reactions were performed in polypropylene 384-well plates in a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μmol/L non-biotinylated affinity ligand) and incubated at room temperature with shaking for 30 minutes. The kinase concentration in the eluate was measured by qPCR. Compound 1 was found to have a K_{d}<10 nM in this assay.

### JAK Family Selectivity Assays

Provided compounds are evaluated for selectivity by comparing their JAK2 binding affinity (K_{d}) in the above JAK2 Binding Assay with their binding affinity (K_{d}) for one or more other kinases. Binding affinity for other kinases is determined as follows: Kinase-tagged T7 phage strains are prepared in an E. coli host derived from the BL21 strain. E. coli are grown to log-phase and infected with T7 phage and incubated with shaking at 32° C. until lysis. The lysates are centrifuged and filtered to remove cell debris. The remaining kinases are produced in HEK-293 cells and subsequently tagged with DNA for qPCR detection. Streptavidin-coated magnetic beads are treated with biotinylated small molecule ligands for 30 minutes at room temperature to generate affinity resins for kinase assays. The liganded beads are blocked with excess biotin and washed with blocking buffer (SeaBlock (Pierce), 1% BSA, 0.05% Tween 20, 1 mM DTT) to remove unbound ligand and to reduce non-specific binding. Binding reactions are assembled by combining kinases, liganded affinity beads, and test compounds in 1× binding buffer (20% SeaBlock, 0.17×PBS, 0.05% Tween 20, 6 mM DTT). Test compounds are prepared as 111× stocks in 100% DMSO. Kds are determined using an 11-point 3-fold compound dilution series with three DMSO control points. All compounds for Kd measurements are distributed by acoustic transfer (non-contact dispensing) in 100% DMSO. The compounds are then diluted directly into the assays such that the final concentration of DMSO is 0.9%. All reactions are performed in polypropylene 384-well plate. Each has a final volume of 0.02 ml. The assay plates are incubated at room temperature with shaking for 1 hour and the affinity beads are washed with wash buffer (1×PBS, 0.05% Tween 20). The beads are then re-suspended in elution buffer (1×PBS, 0.05% Tween 20, 0.5 μM non-biotinylated affinity ligand) and incubated at room temperature with shaking for 30 minutes. The kinase concentration in the eluates is measured by qPCR. Compounds that exhibit a better binding affinity for JAK2 compared to one or more other kinases are considered to be JAK2-selective compounds. In some embodiments, provided compounds may be JAK2-selective over one or more of the following kinases: JAK1, JAK3, and Tyk2.

Results of the JAK2 Selectivity Assay described above are summarized in the following table. Compounds denoted as “A” had a K_{d}/K_{d} ratio≥1000; compounds denoted as “B” had a K_{d}/K_{d} ratio<1000 and ≥300; compounds denoted as “C” had a K_{d}/K_{d} ratio<300 and ≥100.

| | JAK1/JAK2 | JAK3/JAK2 | TYK2/JAK2 |
|---|---|---|---|
| Compound | K_{d}/K_{d} ratio | K_{d}/K_{d} ratio | K_{d}/K_{d} ratio |
| 1 | B | A | C |
| R-1* | C | A | C |

*R-1 is described in WO 2020/097396 (see compound I-2 therein) and has the following structure:

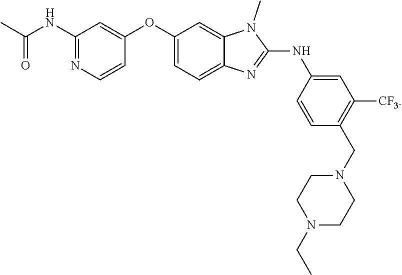 [SMILES: CCN1CCN(Cc2ccc(Nc3nc4ccc(Oc5ccnc(NC(C)=O)c5)cc4n3C)cc2C(F)(F)F)CC1]

### SET2-pSTAT5 Cellular Assay

This assay measures inhibition of JAK2-mediated pSTAT5 signaling in constitutively active essential thrombocytopenia cells carrying the V617F mutation. Cells are harvested from a flask into cell culture medium, and the number of cells is counted. The cells are diluted with culture medium and 100 μL of cell suspension (50000/well) is added into each well of a 96-well cell culture plate. A solution of test compound is added to the assay plate. The plates are covered with a lid and placed in a 37° C. 5% CO₂ incubator for 4 hours. After 4 hours, the cells are spun, and the cell pellets are re-suspended with 100 μL cold PBS. Then, the cells are spun again at 4° C. and 4000 rpm for 5 min. PBS is aspirated, and 25 μL lysis buffer (with protease and phosphatase inhibitor cocktail) is added to each cell pellet. The cell lysate is shaken at 4° C. for 20 min to fully lyse the cells. The cell lysate is spun at 4° C. and 4000 rpm for 15 min, and then the supernatant is transferred into a new plate and stored at −80° C. Meso-scale discovery (MSD) is used to analyze plates as follows: a standard MSD plate is coated with capture antibody in PBS (40 μL/well) and is incubated at 4° C. overnight with shaking. The MSD plate is washed three times with 150 μL/well of 1×MSD Wash Buffer (Tris-buffered saline with 0.1% Tween® 20 detergent, TBST). The MSD plates are then blocked with 150 μL of blocking buffer (5% BSA in TBST) and shaken for 1 h at room temperature and 600 rpm. The MSD plate is washed three times with 150 μL/well of 1×MSD Wash Buffer (TBST). Sample lysates are then added to MSD plates (25 μL/well) and shaken for 1 h at room temperature and 600 rpm. The MSD plate is washed three times with 150 μL/well of 1×MSD Wash Buffer (TBST). Detection antibody (prepared in Antibody Detection buffer, 1% BSA in 1×TBST) is then added to the MSD plates, and they are shaken for 1 h at room temperature and 600 rpm. The MSD plate is washed three times with 150 μL/well of 1×MSD Wash Buffer (TBST). A secondary detection antibody (prepared in Antibody Detection buffer, 1% BSA in 1×TBST) is then added to the MSD plates, and they are shaken for 1 h at room temperature and 600 rpm. The MSD plate is washed three times with 150 μL/well of 1×MSD Wash Buffer (TBST). MSD reading buffer (1×) is added to the plates (150 μL/well), and they are diluted from 4× with water. The plates are imaged using an MSD imaging instrument according to the manufacturer's instructions.

Results of the SET2-pSTAT5 Cellular Assay described above are presented in the following table. Compounds denoted as “A” had a IC₅₀<125 nM; compounds denoted as “C” had a IC₅₀≥200 nM and <1 μM.

| Compound | IC₅₀ |
|---|---|
| 1 | A |
| R-1 | C |

hPBMC-GMCSF-STAT5 Assay

This assay measures inhibition of JAK2-homodimeric-mediated STAT5 signaling in human peripheral blood mononuclear cells. PBMCs are thawed with assay media comprising:

| Reagent | Cat # | Final conc |
|---|---|---|
| RPMI + L-glutamine | Gibco 21870 | 90% |
| Heat Inactivated FBS | Gibco 10082-147 | 10% |
| 1M HEPES | Gibco 15630 | 10 mM |
| 2-mercaptoethanol | Gibco 21985-0231 | 8.6 μL bME/10 mL media |
| Pen/Strep/Glut | Gibco 15140 | 1X |

Then, cells are counted. The cells are diluted with culture medium and 120 μL of cell suspension (500000/well) is added into each well of a 96-well cell culture plate. The test compound is diluted to 10× in assay media, and 15 μL of the solution is added to the assay plates. The plates are covered with a lid and placed in a 37° C., 5% CO₂ incubator for 4 hours. After 4 hours, GM-CSF stock solution (100 g/mL) is diluted to 50 ng/mL in assay media, and 15 μL of the solution is added to the assay plates, such that the final concentration in the assay is 5 ng/mL. The plates are covered with a lid and placed in a 37° C., 5% CO₂ incubator for 5 min. After 5 min, the cells are spun and culture medium aspirated. Then, 50 μL lysis buffer (with protease and phosphatase inhibitor cocktail) is added to each cell pallet, and the cell lysate is shaken at 4° C. for 20 min. The cell lysate is then spun at 4° C., 4000 rpm for 5 min, and the supernatant is transferred into a new plate and stored at −80° C. until further use. An MSD standard plate is coated with capture antibody in PBS (40 μL/well) and incubated at 4° C. overnight with shaking. The MSD plate is then washed three times with 150 μL/well of TBST. Sample lysates (50 μL/well) are added to the MSD plates and shaken for 1 h at RT, 600 rpm. The MSD plates are then washed three times with 150 μL/well of TBST. Detection antibody is added (25 μL/well) and shaken for 1 h at RT, 600 rpm. The detection antibody is prepared in Antibody Detection buffer (1% Blocker A in TBST). The MSD plates are then washed three times with 150 μL/well of TBST. The second detection antibody is added (25 μL/well), shaken for 1 h at RT, 600 rpm. The second detection antibody is prepared in Antibody Detection buffer (1% Blocker A in TBST). The MSD plates are then washed three times with 150 μL/well of TBST. Then, MSD reading buffer (2×) is added (150 μL/well) and diluted from 4× with water. The plates are imaged using an MSD imaging instrument according to the manufacturer's instructions.
hPBMC-IL12-STAT4 Assay

This assay measures inhibition of Tyk2/JAK2-mediated STAT4 signaling in human peripheral blood mononuclear cells. PBMCs are thawed with assay media comprising:

| Reagent | Cat # | Final conc |
|---|---|---|
| RPMI + L-glutamine | Gibco 21870 | 90% |
| Heat Inactivated FBS | Gibco 10082-147 | 10% |
| 1M HEPES | Gibco 15630 | 10 mM |
| 2-mercaptoethanol | Gibco 21985-0231 | 8.6 μL bME/10 mL media |
| Pen/Strep/Glut | Gibco 15140 | 1X |

Then, cells are counted. The cells are diluted with culture medium and 120 μL of cell suspension (200000/well) is added into each well of a 96-well cell culture plate. The test compound is diluted to 10× in assay media, and 15 μL of the solution is added to the assay plates. The plates are covered with a lid and placed in a 37° C., 5% CO₂ incubator for 1 hour. After 1 hour, IL12 stock solution (50 ng/mL) is diluted to 50 ng/mL in assay media, and 15 μL of the solution is added to the assay plates, such that the final concentration in the assay is 1.7 ng/ml. The plates are covered with a lid and placed in a 37° C., 5% CO₂ incubator for 25 min. After 25 min, the cells are spun and culture medium aspirated. Then, 65 μL lysis buffer (with protease and phosphatase inhibitor cocktail) is added to each cell pallet, and the cell lysate is shaken at 4° C. for 30 min. The cell lysate is then spun at 4° C., 4000 rpm for 5 min, and the supernatant is transferred into a new plate and stored at −80° C. until further use. An MSD standard plate is blocked with blocking buffer (3% Blocker A in Wash buffer) and shaken for 1 h at RT, 600 rpm. The MSD plate is then washed three times with 150 μL/well of Wash buffer. Sample lysates (25 μL/well) are added to the MSD plates and shaken for 1 h at RT, 600 rpm. The MSD plates are then washed three times with 150 μL/well of Wash buffer. Detection antibody is added (25 μL/well) and shaken for 1 h at RT, 600 rpm. The detection antibody is prepared in Antibody Detection buffer (for one plate, 150 μL 2% Blocker D-M, 30 μL 10% Blocker D-R, 1 mL of Blocker A solution, 1.82 mL of 1×Wash buffer). The MSD plates are then washed three times with 150 μL/well of TBST. Then, MSD reading buffer (1×) is added (150 μL/well) and diluted from 4× with water. The plates are imaged using an MSD imaging instrument according to the manufacturer's instructions.
hPBMC-IL2-STAT5 Assay

This assay measures inhibition of JAK1/JAK3-mediated STAT5 signaling in human peripheral blood mononuclear cells. PBMCs are thawed with assay media comprising:

| Reagent | Cat # | Final conc |
|---|---|---|
| RPMI + L-glutamine | Gibco 21870 | 90% |
| Heat Inactivated FBS | Gibco 10082-147 | 10% |
| 1M HEPES | Gibco 15630 | 10 mM |
| 2-mercaptoethanol | Gibco 21985-0231 | 8.6 μL bME/10 mL media |
| Pen/Strep/Glut | Gibco 15140 | 1X |

Then, cells are counted. The cells are diluted with culture medium and 120 μL of cell suspension (200000/well) is added into each well of a 96-well cell culture plate. The test compound is diluted to 10× in assay media, and 15 μL of the solution is added to the assay plates. The plates are covered with a lid and placed in a 37° C., 5% CO₂ incubator for 1 hour. After 1 hour, IL2 stock solution (100 μg/mL) is diluted to 250 ng/mL in assay media, and 15 μL of the solution is added to the assay plates, such that the final concentration in the assay is 25 ng/ml. The plates are covered with a lid and placed in a 37° C., 5% CO₂ incubator for 5 min. After 5 min, the cells are spun and culture medium aspirated. Then, 40 μL lysis buffer (with protease and phosphatase inhibitor cocktail) is added to each cell pallet, and the cell lysate is shaken at 4° C. for 20 min. The cell lysate is then spun at 4° C., 4000 rpm for 5 min, and the supernatant is transferred into a new plate and stored at −80° C. until further use. An MSD standard plate is coated with capture antibody in PBS (40 μL/well) and incubated at 4° C. overnight with shaking. The MSD plate is then washed three times with 150 μL/well of TBST. The MSD plates are then blocked with blocking buffer (150 μL of 3% Blocker A in TBST) and shaken for 1 h at RT, 600 rpm. The MSD plate is then washed three times with 150 μL/well of TBST. Sample lysates (40 μL/well) are added to the MSD plates and shaken for 1 h at RT, 600 rpm. The MSD plates are then washed three times with 150 μL/well of TBST. Detection antibody is added (25 μL/well) and shaken for 1 h at RT, 600 rpm. The detection antibody is prepared in Antibody Detection buffer (1% Blocker A in TBST). The MSD plates are then washed three times with 150 μL/well of TBST. The second detection antibody is added (25 μL/well), shaken for 1 h at RT, 600 rpm. The second detection antibody is prepared in Antibody Detection buffer (1% Blocker A in TBST). The MSD plates are then washed three times with 150 μL/well of TBST. Then, MSD reading buffer (2×) is added (150 μL/well) and diluted from 4× with water. The plates are imaged using an MSD imaging instrument according to the manufacturer's instructions.

### Kinome Profiling Assay

Kinome profiling is performed as described in Anastassiadis T, et al. Comprehensive assay of kinase catalytic activity reveals features of kinase inhibitor selectivity. Nat Biotechnol. 2011 Oct. 30; 29(11): 1039-45. doi: 10.1038/nbt.2017. Generally, substrate is prepared in freshly prepared Reaction Buffer (20 mM Hepes (pH 7.5), 10 mM MgCl₂, 1 mM EGTA, 0.01% Brij35, 0.02 mg/mL BSA, 0.1 mM Na₃VO₄, 2 mM DTT, 1% DMSO). Any required cofactors are then added to the substrate solution. Then, the kinase is delivered into the substrate solution and is gently mixed. Test compounds in 100% DMSO are then added to the kinase reaction mixture using Acoustic technology (Echo550; nanoliter range) and incubated for 20 min at RT. 33P-ATP is added to the reaction mixture and incubated for 2 h at RT. Kinase activity is detected by a P981 filter-binding method.

### Caco2 Permeability Assay

Preparation of Caco-2 Cells: 50 μL and 25 mL of cell culture medium are added to each well of a Transwell® insert and reservoir, respectively. Then, the HTS Transwell® plates are incubated at 37° C., 5% CO₂ for 1 hour before cell seeding. Caco-2 cell cells are diluted to 6.86×105 cells/mL with culture medium, and 50 μL of cell suspension are dispensed into the filter well of the 96-well HTS Transwell® plate. Cells are cultivated for 14-18 days in a cell culture incubator at 37° C., 5% CO₂, 95% relative humidity. Cell culture medium is replaced every other day, beginning no later than 24 hours after initial plating.

Preparation of Stock Solutions: 10 mM stock solutions of test compounds arc prepared in DMSO. The stock solutions of positive controls are prepared in DMSO at the concentration of 10 mM. Digoxin and propranolol are used as control compounds in this assay.

Assessment of Cell Monolayer Integrity: Medium is removed from the reservoir and each Transwell® insert and is replaced with prewarmed fresh culture medium. Transepithelial electrical resistance (TEER) across the monolayer is measured using Millicell Epithelial Volt-Ohm measuring system (Millipore, USA). The Plate is returned to the incubator once the measurement is done. The TEER value is calculated according to the following equation: TEER measurement (ohms)×Area of membrane (cm²)=TEER value (ohm·cm²). A TEER value greater than 230 ohm·cm² indicates a well-qualified Caco-2 monolayer.

Assay Procedure: The Caco-2 plate is removed from the incubator and washed twice with pre-warmed HBSS (10 mM HEPES, pH 7.4), and then incubated at 37° C. for 30 minutes. The stock solutions of control compounds are diluted in DMSO to get 1 mM solutions and then diluted with HBSS (10 mM HEPES, pH 7.4) to get 5 μM working solutions. The stock solutions of the test compounds are diluted in DMSO to get 1 mM solutions and then diluted with HBSS (10 mM HEPES and 4% BSA, pH 7.4) to get 5 μM working solutions. The final concentration of DMSO in the incubation system is 0.5%. To determine the rate of drug transport in the apical to basolateral direction. 75 μL of 5 μM working solutions of test compounds are added to the Transwell® insert (apical compartment) and the wells in the receiver plate (basolateral compartment) are filled with 235 μL of HBSS (10 mM HEPES and 4% BSA, pH 7.4). To determine the rate of drug transport in the basolateral to apical direction, 235 μL of 5 μM working solutions of test compounds are added to the receiver plate wells (basolateral compartment) and then the Transwell® inserts (apical compartment) are filled with 75 μL of HBSS (10 mM HEPES and 4% BSA, pH 7.4). Time 0 samples are prepared by transferring 50 μL of 5 μM working solution to wells of the 96-deepwell plate, followed by the addition of 200 μL cold methanol containing appropriate internal standards (IS). The plates are incubated at 37° C. for 2 hours. At the end of the incubation, 50 μL samples from donor sides (apical compartment for Ap→Bl flux, and basolateral compartment for Bl→Ap) and receiver sides (basolateral compartment for Ap→Bl flux, and apical compartment for Bl→Ap) are transferred to wells of a new 96-well plate, followed by the addition of 4 volume of cold acetonitrile or methanol containing appropriate internal standards (IS). Samples are vortexed for 5 minutes and then centrifuged at 3,220 g for 40 minutes. An aliquot of 100 μL of the supernatant is mixed with an appropriate volume of ultra-pure water before LC-MS/MS analysis. To determine the Lucifer Yellow leakage after 2 hour transport period, stock solution of Lucifer yellow is prepared in ultra-pure water and diluted with HBSS (10 mM HEPES, pH 7.4) to reach the final concentration of 100 μM. 100 μL of the Lucifer yellow solution is added to each Transwell® insert (apical compartment), followed by filling the wells in the receiver plate (basolateral compartment) with 300 μL of HBSS (10 mM HEPES, pH 7.4). The plates are incubated at 37° C. for 30 minutes. 80 μL samples are removed directly from the apical and basolateral wells (using the basolateral access holes) and transferred to wells of new 96 wells plates. The Lucifer Yellow fluorescence (to monitor monolayer integrity) signal is measured in a fluorescence plate reader at 485 nM excitation and 530 nM emission.

Results of the Caco-2 Permeability Assay described above are presented in the following table. Compounds denoted as “B” had a ER>2 and ≤5; compounds denoted as “D” had a ER>10 and ≤30.

| | Papp | Efflux Ratio |
|---|---|---|
| Compound | (A-B, 10⁻⁶ cm/s) | (ER) |
| 1 | 7 | B |
| R-1 | 0.4 | D |

### Cytotoxicity Assay

HEK293T cells are harvested from flask into cell culture medium, and then the cells are counted. The cells are diluted with culture medium to the desired density, and 40 μL of cell suspension is added into each well of a 384-well cell culture plate. The plates are covered with a lid and spun at room temperature at 1,000 RPM for 1 minute and then transferred into 37° C. 5% CO₂ incubator overnight. Test compounds are dissolved at 10 mM DMSO stock solution. 45 μL of stock solution is then transferred to a 384 PP-plate. A 3-fold, 10-point dilution is performed via transferring 15 μL compound into 30 μL DMSO by using TECAN (EVO200) liquid handler. The plates are spun at room temperature at 1,000 RPM for 1 minute and shaken on a plate shaker for 2 minutes. 40 nL of diluted compound is transferred from compound source plate into the cell plate by using liquid handler Echo550. After compound treatment for 48 hours, CTG detection is performed for compound treatment plates: the plates are removed from incubators and equilibrated at room temperature for 15 minutes. 30 μL of CellTiter-Glo reagent is added into each well to be detected. The plates are then placed at room temperature for 30 min followed by reading on EnVision. Inhibition activity is calculated with the following formula: % Inhibition=100×(LumHC−LumSample)/(LumHC−LumLC), wherein HC is reading obtained from cells treated with 0.1% DMSO only and LC is reading from cells treated with 10 μL staurosporine. IC₅₀ values are calculated using XLFit (equation 201).

### Hepatocyte Stability Assay

10 mM stock solutions of test compound and positive control are prepared in DMSO. Stock solutions are diluted to 100 μM by combining 198 μL of 50% acetonitrile/50% water and 2 μL of 10 mM stock solution. Verapamil is used as positive control in the assay. Vials of cryopreserved hepatocytes are thawed in a 37° C. water bath with gently shaking. The contents are poured into the 50 mL thawing medium conical tube. Vials are centrifuged at 100 g for 10 minutes at room temperature. Thawing medium is aspirated and hepatocytes are re-suspended with serum-free incubation medium to yield ˜1.5×106 cells/mL. Cell viability and density are counted using a Trypan Blue exclusion, and then cells are diluted with serum-free incubation medium to a working cell density of 0.5×106 viable cells/mL. A portion of the hepatocytes at 0.5×106 viable cells/mL are boiled for 5 min prior to adding to the plate as negative control to eliminate the enzymatic activity so that little or no substrate turnover should be observed. Aliquots of 198 μL hepatocytes are dispensed into each well of a 96-well non-coated plate. The plate is placed in the incubator for approximately 10 minutes. Aliquots of 2 μL of the 100 μM test compound and 2 μL positive control are added into respective wells of a non-coated 96-well plate to start the reaction. The final concentration of test compound is 1 μM. This assay is performed in duplicate. The plate is incubated in the incubator for the designed time points. 25 μL of contents are transferred and mixed with 6 volumes (150 μL) of cold acetonitrile with internal standard (100 nM alprazolam, 200 nM labetalol, 200 nM caffeine and 200 nM diclofenac) to terminate the reaction at time points of 0, 15, 30, 60, 90 and 120 minutes. Samples are centrifuged for 25 minutes at 3,220 g and aliquots of 150 μL of the supernatants are used for LC-MS/MS analysis.

Results of the Hepatocyte Stability Assay described above are presented in Table 6, with human or rat hepatocytes. For human heps CLᵢₙₜ: compounds denoted as “A” had a CLᵢₙₜ≤6 mL/min/kg; compounds denoted as “C” had a CLᵢₙₜ>12 mL/min/kg and <20 mL/min/kg. For rat heps CLᵢₙₜ: compounds denoted as “B” had a CLᵢₙₜ≥17 mL/min/kg and <35 mL/min/kg; compounds denoted as “C” had a CLᵢₙₜ≥35 mL/min/kg and <45 mL/min/kg.

| | Human | Rat Heps |
|---|---|---|
| Compound | Heps CLᵢₙₜ | CLᵢₙₜ |
| 1 | A | B |
| R-1 | C | C |

### Kinetic Solubility Assay

Stock solutions of test compounds are prepared in DMSO at the concentration of 10 mM, and a stock solution of control compound is prepared in DMSO at the concentration of 30 mM. Diclofenac is used as positive control in the assay. 30 μL stock solution of each compound is placed into their a 96-well rack, followed by adding 970 μL of PBS at pH 4.0 and pH 7.4 into each vial of the cap-less solubility sample plate. This study is performed in duplicate. One stir stick is added to each vial and then vials are sealed using a molded PTDE/SIL 96-Well Plate Cover. The solubility sample plate is transferred to the Thermomixer comfort plate shaker and incubated at RT for 2 hours with shaking at 1100 rpm. After 2 hours incubation, stir sticks are removed using a big magnet and all samples from the solubility sample plate are transferred into the filter plate. All the samples are filtered by vacuum manifold. The filtered samples are diluted with methanol. Samples are analyzed by LC-MS/MS and quantified against a standard of known concentration in DMSO using LC coupled with Mass spectral peak identification and quantitation. The solubility values of the test compounds are calculated as follows, wherein INJ VOL is injection volume, DF is dilution factor, and STD is standard:

$$[ Sample ] = \frac{{AREA}_{Sample} \times I N J V O {L}_{S t d} \times D {F}_{Sample} \times [ S T D ]}{{AREA}_{S t d} \times I N J V O {L}_{Sample}}$$

Results of the Kinetic Solubility Assay described above are presented in Table 7. Compounds denoted as “B” had a solubility ≥9 UM and <100 μM; compounds denoted as “C” had a solubility ≥100 μM and <200 μM.

| Compound | Solubility |
|---|---|
| 1 | B |
| R-1 | C |

### Plasma Protein Binding Assay

Working solutions of test compounds and control compound are prepared in DMSO at the concentration of 200 μM, and then the working solutions are spiked into plasma. The final concentration of compound is 1 μM. The final concentration of DMSO is 0.5%. Ketoconazole is used as positive control in the assay. Dialysis membranes are soaked in ultrapure water for 60 minutes to separate strips, then in 20% ethanol for 20 minutes, finally in dialysis buffer for 20 minutes. The dialysis set up is assembled according to the manufacturer's instruction. Each Cell is with 150 μL of plasma sample and dialyzed against equal volume of dialysis buffer (PBS). The assay is performed in duplicate. The dialysis plate is sealed and incubated in an incubator at 37° C. with 5% CO₂ at 100 rpm for 6 hours. At the end of incubation, 50 μL of samples from both buffer and plasma chambers are transferred to wells of a 96-well plate. 50 μL of plasma is added to each buffer samples and an equal volume of PBS is supplemented to the collected plasma sample. 400 μL of precipitation buffer acetonitrile containing internal standards (IS, 100 nM alprazolam, 200 nM labetalol, 200 nM imipramine and 2 μM ketoplofen) is added to precipitate protein and release compounds. Samples are vortexed for 2 minutes and centrifuged for 30 minutes at 3,220 g. Aliquot of 50 μL of the supernatant is diluted by 150 μL acetonitrile containing internal standards: ultra-pure H₂O=1:1, and the mixture is used for LC-MS/MS analysis.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A crystalline solid form of Compound 1:

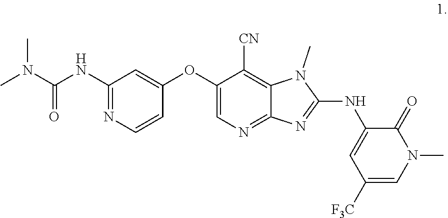

2.-6. (canceled)

7. The solid form of claim 1, wherein the solid form is an anhydrate.

8. The solid form of claim 7, wherein the solid form is characterized by three or more peaks in its XRPD pattern selected from those at angles (degrees 2 theta±0.2) of 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67.

9. The solid form of claim 8, wherein the solid form is characterized by peaks in its XRPD pattern selected from those at angles (degrees 2 theta±0.2) of 9.05, 9.09, 10.16, 12.85, 14.42, 15.10, 16.39, 17.08, 17.66, 19.35, 19.85, 20.35, 21.89, 22.83, 23.71, 24.20, 25.09, 27.10, 27.57, 28.59, and 28.67.

10. The solid form of claim 8, wherein the solid form is characterized by three or more peaks in its XRPD pattern selected from those at angles (degrees 2 theta±0.2) of 9.05, 9.09, 10.16, 12.85, 15.10, and 19.35.

11. The solid form of claim 10, wherein the solid form is characterized by peaks in its XRPD pattern selected from those at angles (degrees 2 theta±0.2) of 9.05, 9.09, 10.16, 12.85, 15.10, and 19.35.

12. The solid form of claim 11, wherein the solid form is characterized by peaks in its XRPD pattern comprising those at substantially all of the following angles (degrees 2 theta #0.2):
6.40
9.05
9.09
10.16
12.85
14.42
15.10
15.79
16.39
17.08
17.66
18.28
18.72
19.35
19.85
20.35
21.89
22.83
23.37
23.71
24.20
25.09
26.73
27.10
27.57
28.59
28.67
29.06
29.37
29.75
30.49
30.85
31.92
32.59
32.87.

13. The solid form of claim 7, wherein the solid form is characterized by an XRPD pattern substantially similar to that depicted in FIG. 6.

14.-89. (canceled)

90. A method of inhibiting JAK2 in a subject comprising administering to the subject the solid form of claim 1.

91. A method of treating a disease, disorder, or condition associated with JAK2, comprising administering to a subject in need thereof the solid form of claim 1.

92. A method of treating cancer, comprising administering to a subject in need thereof the solid form of claim 1.

93. A method of treating a hematological malignancy, comprising administering to a subject in need thereof the solid form of claim 1.

94. The method of claim 93, wherein the hematological malignancy is leukemia or lymphoma.

95. A method of treating a myeloproliferative neoplasm, comprising administering to a subject in need thereof the solid form of claim 1.

96. The method of claim 95, wherein the myeloproliferative neoplasm is polycythemia vera, essential thrombocytopenia or myelofibrosis.
